(19) United States
(12) Patent Application Publication
(10) Pub. No.: US 2024/0067983 A1
(43) Pub. Date: Feb. 29, 2024

(54) Guide RNA Designs and Complexes for Tracr-less Type V Cas Systems

(71) Applicant: Horizon Discovery Limited, Waterbeach, Cambridge (GB)

(72) Inventors: Kurt Daniel Marshall, Erie, CO (US); Hide Bueno Machado, Arvada, CO (US); Emily Anderson, Arvada, CO (US); Alexander Hale, Longstanton, Cambridgeshire (GB); Amanda Smith, Trumpington, Cambridge (GB); Anastasia Kaufman, Thornton, CO (US); Leah Nantie, Arvada, CO (US); Michael Daniel Rushton, Hitchin, Hertfordshire (GB); Kevin Hemphill, Erie, CO (US)

(21) Appl. No.: 18/270,740

(22) PCT Filed: Jan. 5, 2022
(86) PCT No.: PCT/US2022/011289

§ 371 Date: Jul. 3, 2023

Related U.S. Application Data
(60) Provisional application No. 63/133,942, filed on Jan. 5, 2021.

(51) Int. Cl. C12N 15/85; C12N 5/071; C12N 9/22; C12N 9/78; C12N 15/11

(52) U.S. Cl. CPC C12N 15/85; C12N 5/0686; C12N 9/22; C12N 9/78; C12N 15/11; C12Y 305/04; C12N 2310/20; C12N 2310/531

(57) ABSTRACT

A novel gRNA-ligand binding complex is provided. This complex may be used to bring Type V Cas proteins and additional effectors to DNA for base editing. The design of the systems allows for the production of efficient modular components that provide flexibility when editing DNA.

### CROSS-REFERENCE TO RELATED APPLICATION

This application is a national stage application of international application serial number PCT/US2022/011289, filed Jan. 5, 2022, which claims the benefit of the filing date of U.S. Provisional Application Ser. No. 63/133,942, filed Jan. 5, 2021, the entire disclosures of which are incorporated by reference as if set forth fully herein.

### FIELD OF THE INVENTION

The present invention relates to the field of gene-editing.

### BACKGROUND OF THE INVENTION

Researchers are aggressively exploring the use of Clustered Regularly Interspaced Short Palindromic Repeat (CRISPR) systems in order to modify DNA. To date, the vast majority of the work in this field has been in Cas9 systems. In these systems, a tracrRNA (trans-activating CRISPR RNA) and a crRNA (CRISPR RNA) hybridize to recruit a Cas9 protein and then direct the Cas9 protein to a DNA location that is complementary to a sequence within the crRNA. The complementary sequence within the DNA thus becomes a target site, and the Cas9 protein may, based on its functional domain, cause editing at this target site.

Despite the now well-recognized power of the Cas9 systems, those systems are not effective in all applications. Among the limitations of Cas9 systems are that the functional domains upon which the Cas9 systems can act are defined by the functional domain of the Cas9 protein that one uses and that the use of both a tracrRNA and a crRNA can be cumbersome.

Other Cas proteins are known. Among these other Cas proteins, the potential of which has not been fully explored, are those within the Type V family, particularly those that do not require the presence of a tracrRNA to function. Within these systems, one may use a single guide RNA (gRNA) that contains a crRNA sequence. This crRNA sequence can associate with the Cas protein of interest without needing to be associated with a tracrRNA. The absence of a need for a tracrRNA provides an underexplored possibility of developing improved gRNAs as well as complexes and systems that incorporate and use them.

### SUMMARY OF THE INVENTION

The present invention provides novel and non-obvious gRNA-ligand binding complexes, base editing complexes, and methods for base editing. Through the use of various embodiments of the present invention, one may be able to efficiently and effectively cause base editing ex vivo, in vitro, and in vivo. Further, some embodiments of the present invention provide modular designs that allow for the same Type V Cas protein to be directed to different targeting sites and optionally associated with different effector proteins at the same or different sites.

According to a first embodiment, the present invention provides a gRNA-ligand binding complex, wherein the gRNA-ligand binding complex comprises: (a) a gRNA, wherein the gRNA is 35 to 60 or 36 to 60 nucleotides long and the gRNA has a crRNA sequence, wherein the crRNA sequence is 35 to 60 or 36 to 60 nucleotides long and the crRNA sequence comprises a Cas association region, wherein the Cas association region is 14 to 37 or 18 to 30 nucleotides long and a targeting region, wherein the targeting region is 14 to 37 or 18 to 30 or 18 to 20 nucleotides long and the Cas association region is capable of retaining association with an RNA binding domain of a Type V Cas protein in the absence of a tracrRNA; and (b) a ligand binding moiety, wherein the ligand binding moiety is either (i) directly bound to the gRNA, or (ii) bound to the gRNA through a linker. In one embodiment, the gRNA of the gRNA-ligand binding complex comprises or consists essentially of a chemically modified or unmodified sequence that is or encodes SEQ ID NO: 137.

According to a second embodiment, the present invention provides a base editing complex comprising: a gRNA-ligand binding complex of the present invention and a Type V Cas protein, wherein the Cas association region of the gRNA-ligand binding complex is associated with the Type V Cas protein. Optionally, the ligand binding moiety is reversibly associated with a ligand that is attached to or a part of an effector molecule.

According to a third embodiment, the present invention provides a method for base editing. The method comprises exposing a base editing complex of the present invention to double stranded DNA (“dsDNA”) or single stranded DNA (“ssDNA”). The base editing complex may be exposed to the dsDNA or ssDNA under conditions that permit base editing.

When an effector is attached to (or contains) a ligand, the system has a modular design. The presence of the ligand binding moiety within the gRNA-ligand binding complex allows that complex to associate with the corresponding ligand associated with (or contained within) the effector. Thus, the ligand binding moiety is associated with the gRNA in a manner and orientation that allows it to be capable of associating with a ligand. Similarly, the ligand is attached to or associated with the effector in a manner that renders it capable of reversibly associating with the ligand binding moiety.

When the ligand and the ligand binding moiety are associated with each other, the effector that is associated with the ligand will become part of any base editing complex that contains the gRNA-ligand binding complex. When the base editing complex also contains a Cas protein, that Cas protein and the effector can be retained in the same locality, e.g., at or near a target site of interest.

Thus, if one wishes to use a particular effector with the Cas protein, one only needs to associate that effector with the ligand that is capable of reversibly associating with the ligand binding moiety that is part of the base editing complex that contains that Cas protein. To change the effector from one system to the next, one need only change the effector-ligand. Consequently, one can use the same gRNA-ligand binding complex and its associated Cas protein with a plurality of different effectors. The plurality of different effectors may be used sequentially in the same system by associating and dissociating their ligands with the ligand binding moieties or simultaneously or sequentially in different systems.

### BRIEF DESCRIPTION OF THE FIGS.

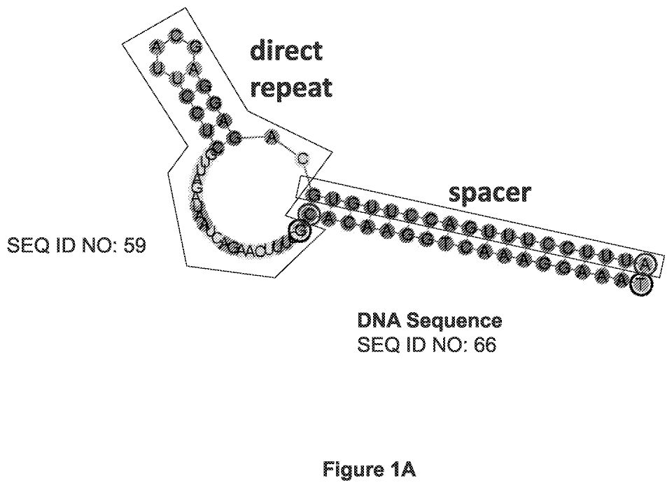

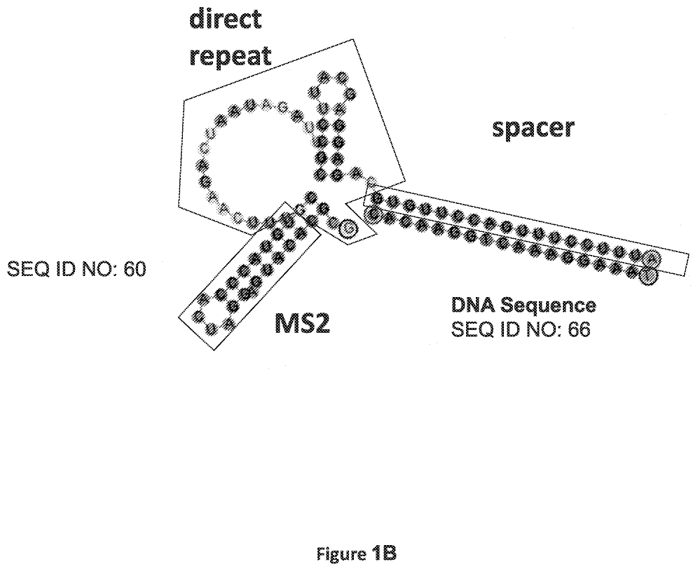

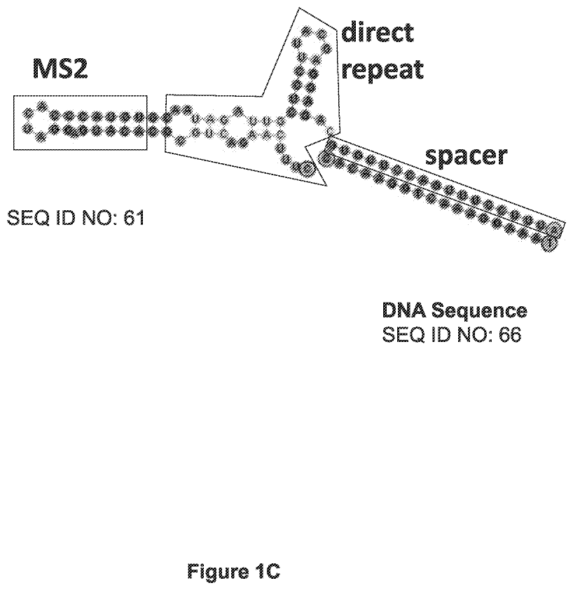

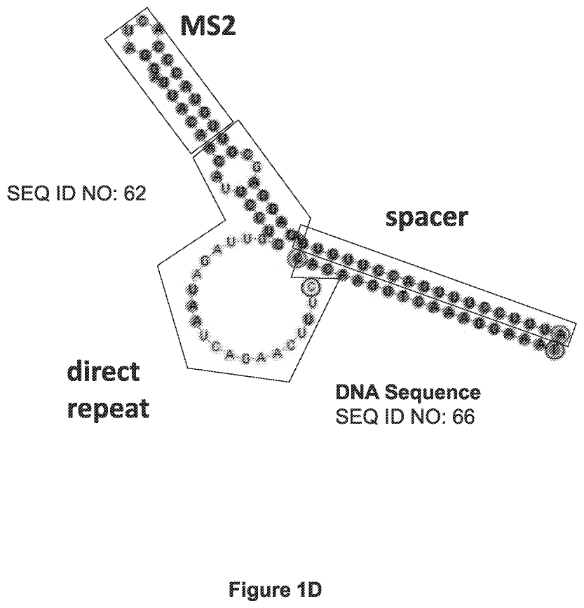

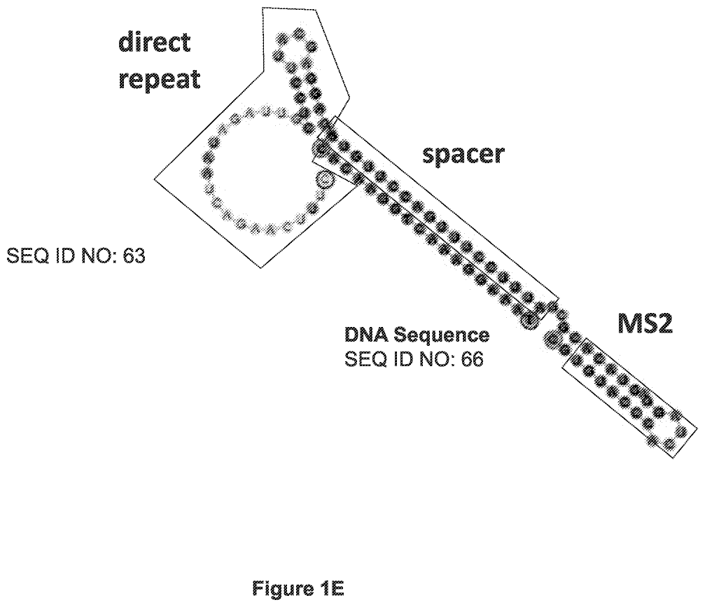

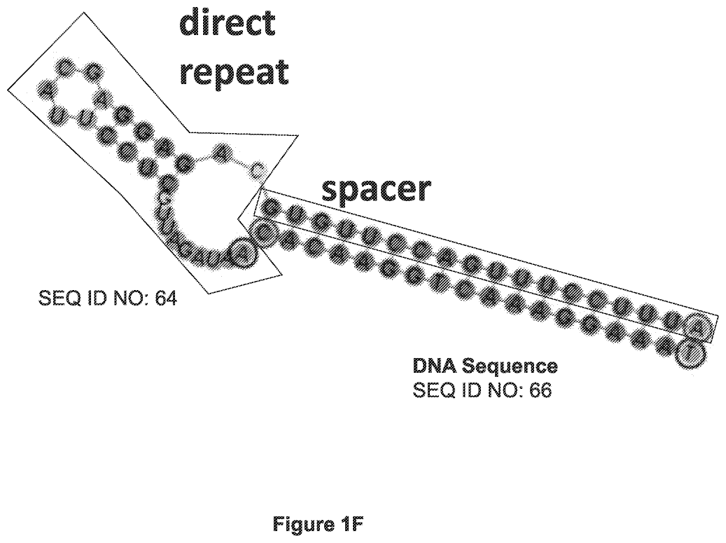

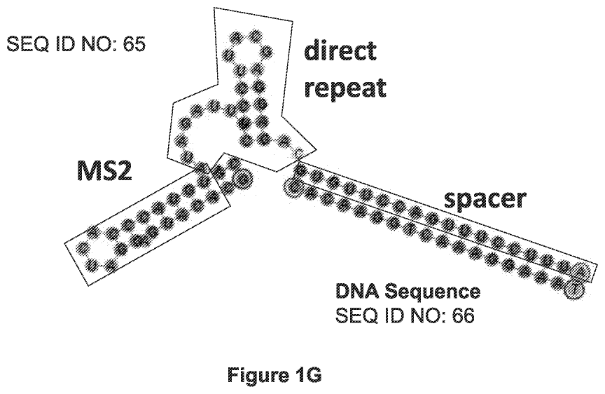

FIGS. 1A to FIG. 1G are representations of examples of CasPhi guide RNAs that show direct repeat, spacer, and when present, MS2 ligand binding moiety locations. The gRNAs are shown bound to a DNA strand (SEQ ID NO: 66) at the spacer regions.

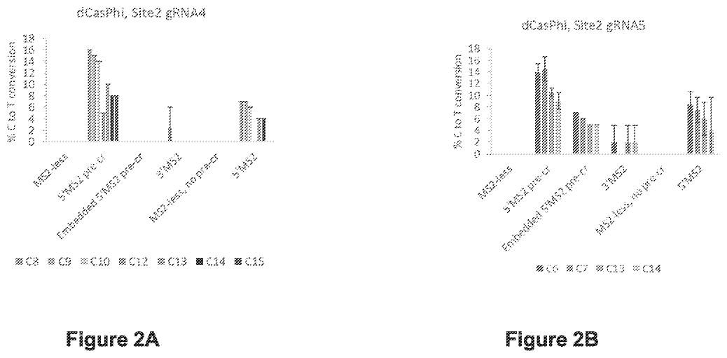

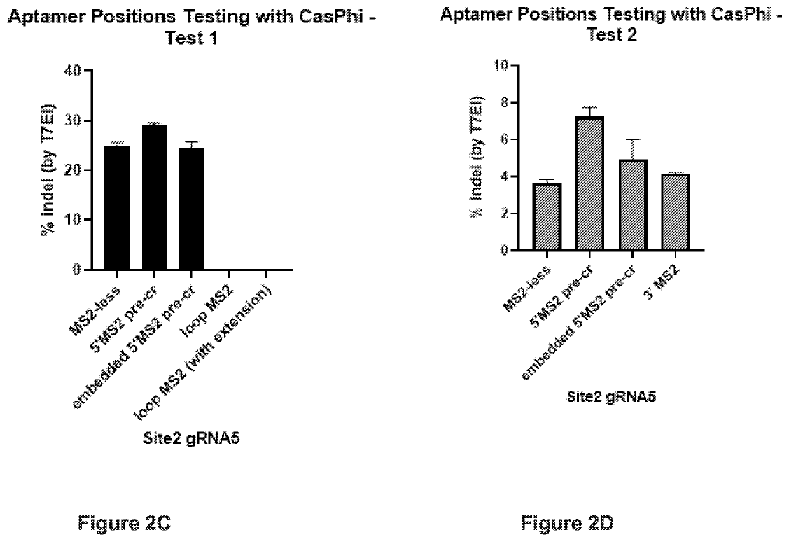

FIG. 2A and FIG. 2B are bar graphs that depict the effects of MS2 placement on dCasPhi base editing levels for two genomic target sites. FIG. 2C and FIG. 2D are representations of the evaluation of the effects of MS2 placement on gene disruption levels with CasPhi.

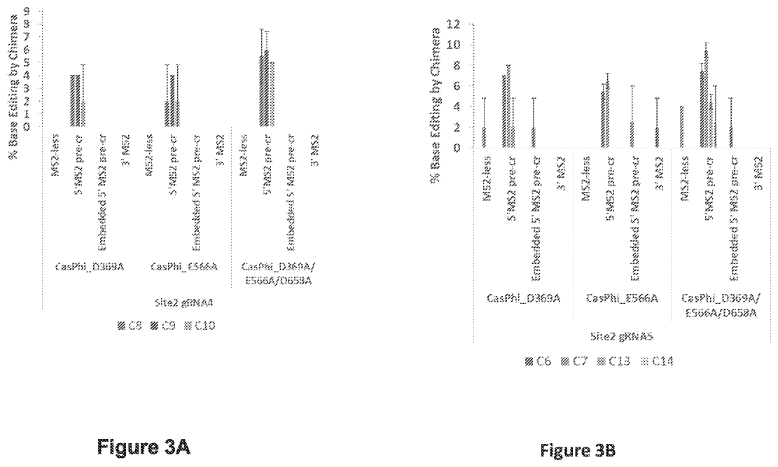

FIG. 3A and FIG. 3B are bar graphs that depict base editing with guides for multiple sets of deactivated CasPhi mutants.

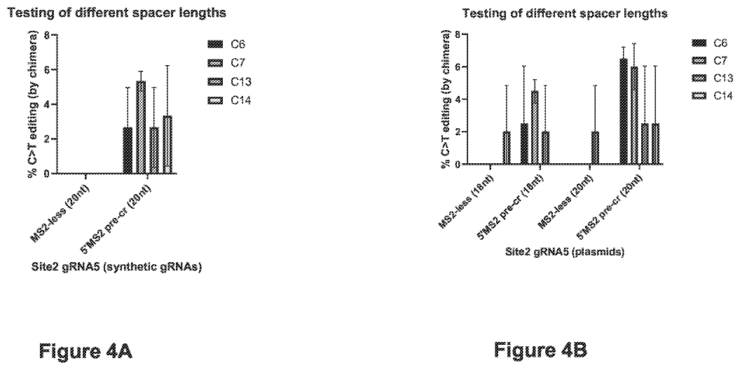

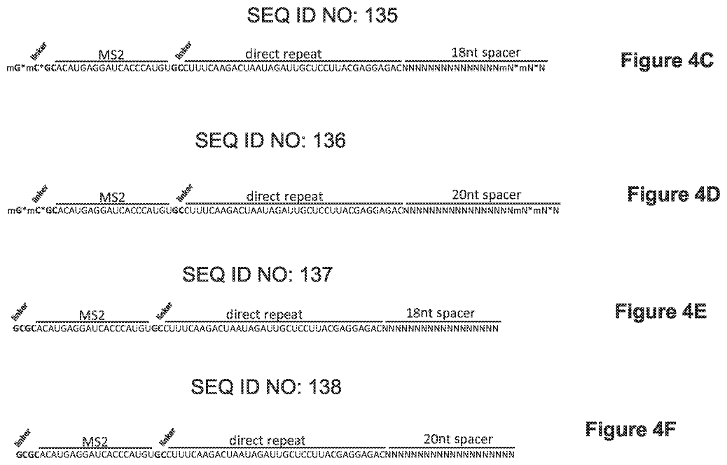

FIG. 4A and FIG. 4B are bar graphs that summarize dCasPhi base editing efficiency when different length spacers for expressed gRNAs are used. FIGS. 4C, 4D, 4E, and 4F are schematics of gRNAs with different spacer lengths.

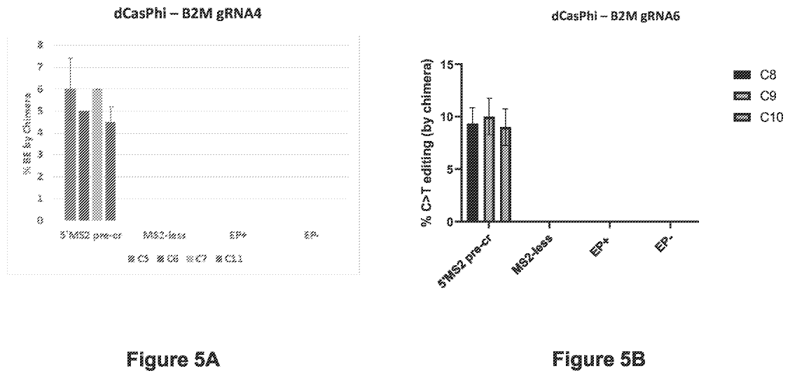

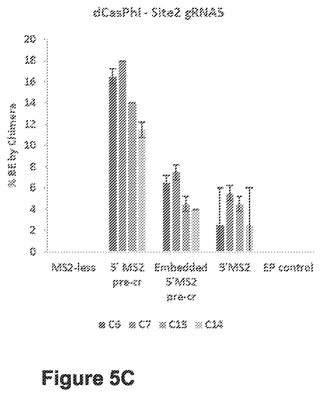

FIG. 5A, FIG. 5B, and FIG. 5C are bar graphs that depict dCasPhi base editing at multiple sites in HEK293T cells with chemically synthesized and chemically modified guides.

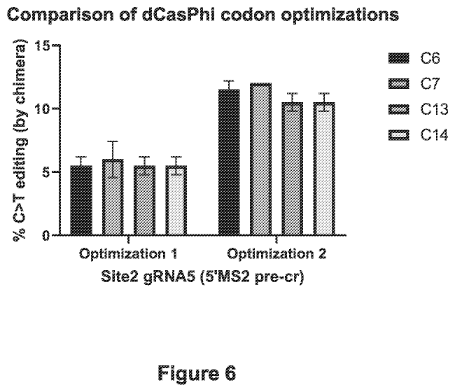

FIG. 6 is a bar graph representation of two different codon optimized dCasPhi base editing at HEK Site2 site in HEK293T cells with chemically modified synthetic guides.

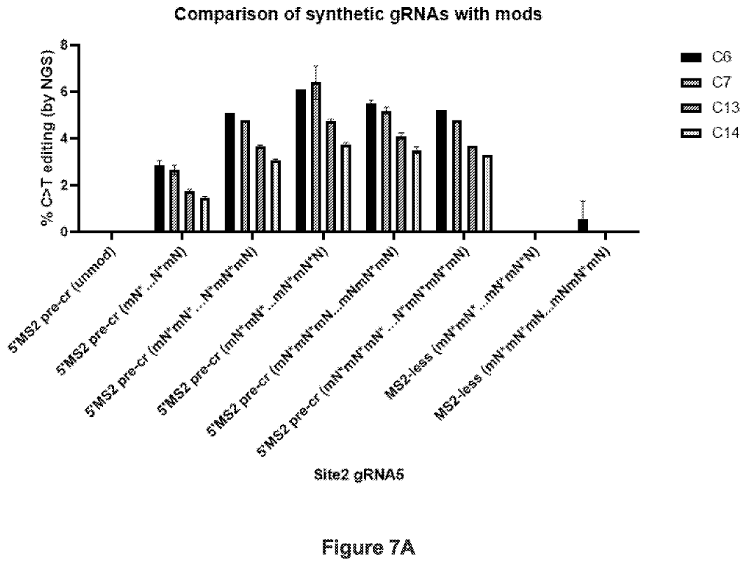

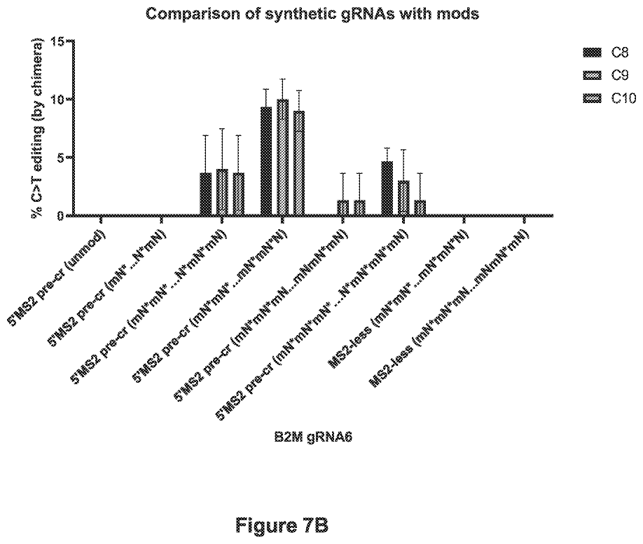

FIG. 7A and FIG. 7B are bar graph representations of the effects of synthetic guide chemical modifications on dCasPhi base editing levels at two different genomic target sites in HEK293T cells.

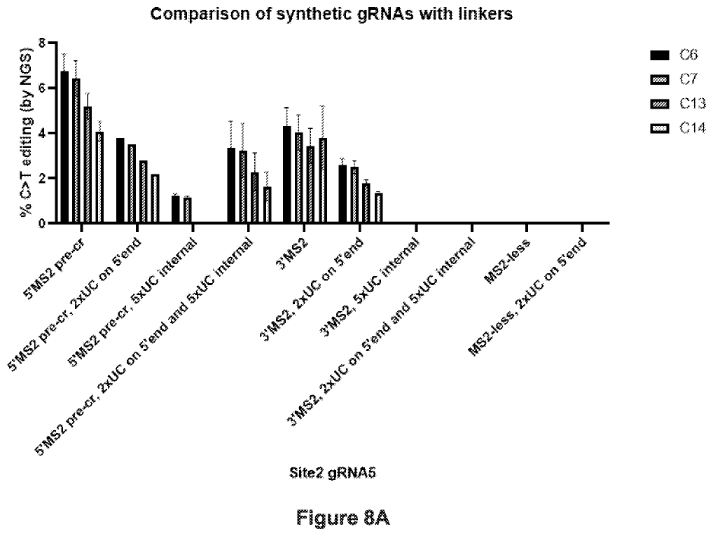

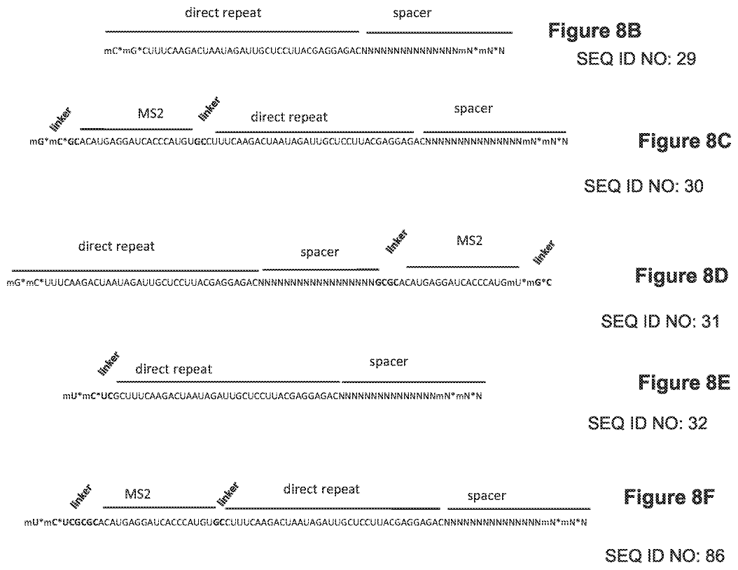

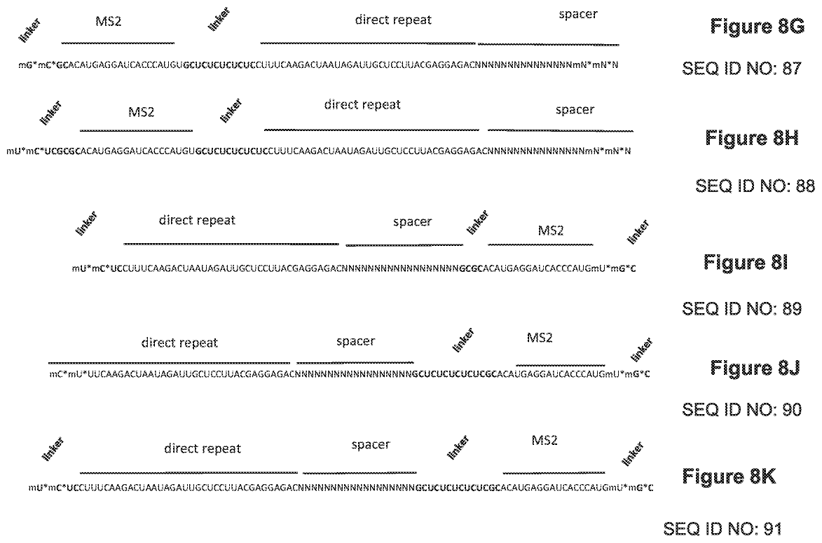

FIG. 8A is a bar graph representation of an assessment of the effects of linkers in gRNA sequences on dCasPhi base editing levels. FIG. 8B is a schematic of a gRNA with no linkers. FIGS. 8C to 8K are schematics of gRNA sequences with different locations and combinations of linkers.

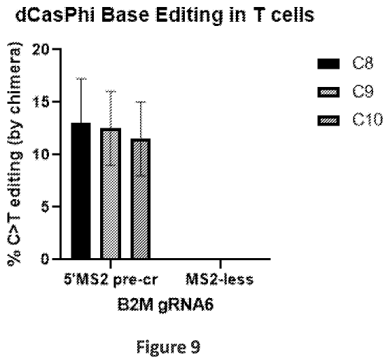

FIG. 9 is a bar graph representation of dCasPhi aptamer-recruitment base editing in T Lymphocytes.

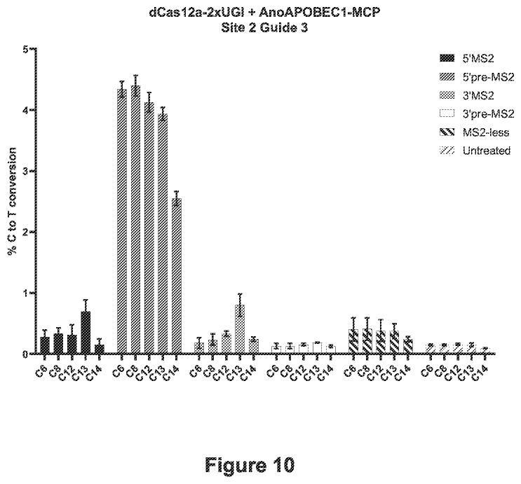

FIG. 10 is a bar graph representation of dCas12a base editing at HEK Site2 site in HEK293T cells with chemically modified guides.

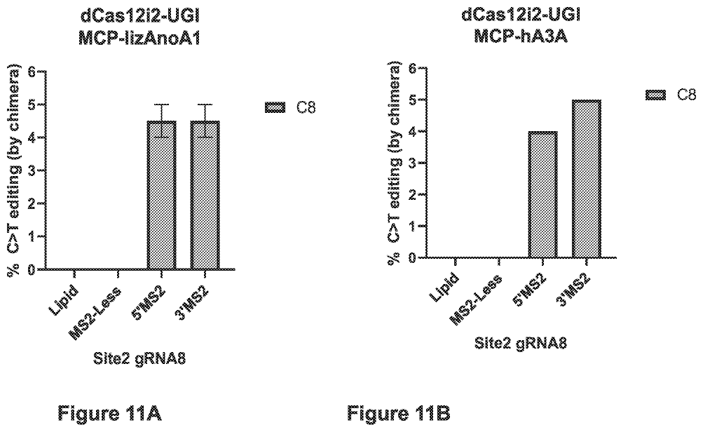

FIG. 11A and FIG. 11B are representation of the evaluation of the effects of dCas12i2 base editing at HEK Site2 site with multiple deaminases in HEK293T cells.

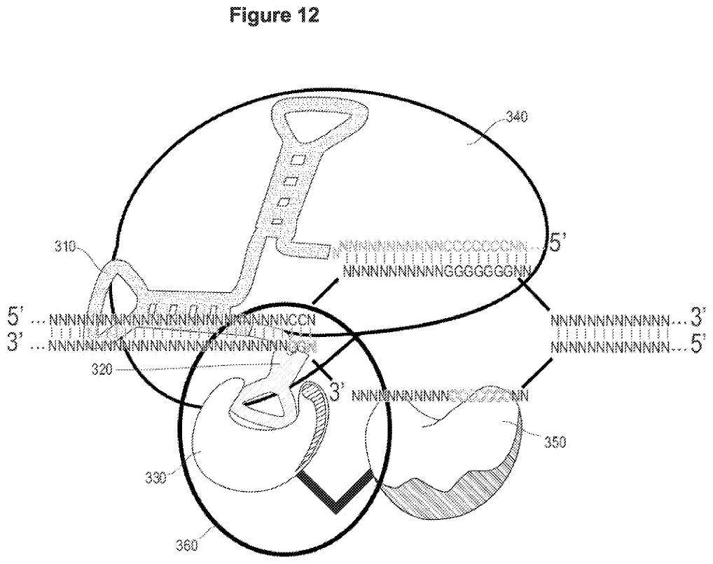

FIG. 12 is a representation of a base editing complex of the present invention.

### DETAILED DESCRIPTION OF THE INVENTION

Reference will now be made in detail to various embodiments of the present invention, examples of which are illustrated in the accompanying figures. In the following description, numerous specific details are set forth in order to provide a thorough understanding of the present invention. However, unless otherwise indicated or implicit from context, the details are intended to be examples and should not be deemed to limit the scope of the invention in any way. Additionally, features described in connection with the various or specific embodiments are not to be construed as not appropriate for use in connection with other embodiments disclosed herein unless such exclusivity is explicitly stated or implicit from context.

Headers are provided herein for the convenience of the reader and do not limit the scope of any of the embodiments disclosed herein.

Definitions

Unless otherwise stated or apparent from context, the following terms shall have the meanings set forth below:

The phrase “2′ modification” refers to a nucleotide unit having a sugar moiety that is modified at the 2′ position of the sugar moiety. An example of a 2′ modification is a 2′-O-alkyl modification that forms a 2′-O-alkyl modified nucleotide or a 2′ halogen modification that forms a 2′ halogen modified nucleotide.

The phrase “2′-O-alkyl modified nucleotide” refers to a nucleotide unit having a sugar moiety, for example a, deoxyribosyl or ribosyl, moiety that is modified at the 2′ position such that an oxygen atom is attached both to the carbon atom located at the 2′ position of the sugar and to an alkyl group. In various embodiments, the alkyl moiety consists of or consists essentially of carbon(s) and hydrogens. When the O moiety and the alkyl group to which it is attached are viewed as one group, they may be referred to as an O-alkyl group, e.g., —O-methyl, —O-ethyl, —O-propyl, —O-isopropyl, —O-butyl, —O-isobutyl, —O-ethyl-O-methyl (—OCH₂CH₂OCH₃), and —O-ethyl-OH (—OCH₂CH₂OH). A 2′-O-alkyl modified nucleotide may be substituted or unsubstituted.

The phrase “2′ halogen modified nucleotide” refers to a nucleotide unit having a sugar moiety, for example a deoxyribosyl moiety that is modified at the 2′ position such that the carbon at that position is directly attached to a halogen species, e.g., Fl, Cl, or Br.

A “ligand binding moiety” refers to a moiety such as an aptamer e.g., oligonucleotide or peptide or another compound that binds to a specific ligand and can reversibly or irreversibly be associated with that ligand.

The term “modified nucleotide” refers to a nucleotide having at least one modification in the chemical structure of the base, sugar and/or phosphate, including, but not limited to, 5-position pyrimidine modifications, 8-position purine modifications, modifications at cytosine exocyclic amines, and substitution of 5-bromo-uracil or 5-iodouracil; and 2′-modifications, including but not limited to, sugar-modified ribonucleotides in which the 2′-OH is replaced by a group such as an H, OR, R, halo, SH, SR, NH₂, NHR, NR₂, or CN, wherein R is an alkyl.

Modified bases refer to nucleotide bases such as, for example, adenine, guanine, cytosine, thymine, uracil, xanthine, inosine, and queuosine that have been modified by the replacement or addition of one or more atoms or groups. Some examples of these types of modifications include, but are not limited to, alkylated, halogenated, thiolated, aminated, amidated, or acetylated bases, alone and in various combinations. More specific modified bases include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyluridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O— and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4-thioribose, and other sugars, heterocycles, or carbocycles.

The phrase “codes for” and the term “encodes” mean that one sequence contains either a sequence that is identical to a referenced nucleotide sequence, a DNA or RNA equivalent of the referenced nucleotide sequence, or a DNA or RNA or a sequence that is a DNA or RNA complement of the referenced nucleotide sequence. Thus, when one refers to a sequence that codes for or encodes a recited DNA sequence, one refers to a sequence that unless otherwise specified is any one of the following: the same DNA sequence, a complement of the DNA sequence, the RNA equivalent of that sequence, or the RNA complement of that sequence or any of the aforementioned in which one or more ribonucleotides is substituted for its deoxyribonucleotide counterpart or one or more deoxyribonucleotides is substituted for its ribonucleotide counterpart.

The term “complementarity” refers to the ability of a nucleic acid to form one or more hydrogen bonds with another nucleic acid sequence by either traditional Watson-Crick base-pairing or other non-traditional types. A percent complementarity indicates the percentage of residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, at least 65%, at least 70%, at least 75%, at least 80%, at least 85%, at least 90%, at least 95%, at least 97%, at least 98%, or at least 99%, over a region of for example, 8, 9, 10, 11, 12, 13, 14, 15, 16, 17, 18, 19, 20, 21, 22, 23, 24, 25, 30, 35, 40, 45, 50, or more consecutive nucleotides, or refers to two nucleic acids that hybridize under stringent conditions.

The terms “hybridization” and “hybridizing” refer to a process in which completely, substantially, or partially complementary nucleic acid strands come together under specified hybridization conditions to form a double-stranded structure or region in which the two constituent strands are joined by hydrogen bonds. Unless otherwise stated, the hybridization conditions are naturally occurring or lab designed conditions. Although hydrogen bonds typically form between adenine and thymine or uracil (A and T or U) or between cytidine and guanine (C and G), other base pairs may form (see e.g., Adams et al., The Biochemistry of the Nucleic Acids, 11th ed., 1992).

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Preferably, a nucleotide comprises a cytosine, uracil, thymine, adenine, or guanine moiety. Further, the term nucleotide also includes those species that have a detectable label, such as for example a radioactive or fluorescent moiety, or mass label attached to the nucleotide. The term nucleotide also includes what are known in the art as universal bases. By way of example, universal bases include but are not limited to 3-nitropyrrole, 5-nitroindole, or nebularine. Nucleotide analogs are, for example, meant to include nucleotides with bases such as inosine, queuosine, xanthine, sugars such as 2′-methyl ribose, and non-natural phosphodiester internucleotide linkages such as methylphosphonates, phosphorothioates, phosphoroacetates and peptides.

The terms “subject” and “patient” are used interchangeably herein to refer to an organism. e.g., a vertebrate, preferably a mammal, more preferably a human Mammals include, but are not limited to, murines, simians, humans, farm animals, sport animals, and pets such as dogs and cats. The tissues, cells and their progeny of an organism or other biological entity obtained in vivo or cultured in vitro are also encompassed within the terms subject and patient. Additionally, in some embodiments, a subject may be an invertebrate animal, for example, an insect or a nematode; while in others, a subject may be a plant or a fungus.

As used herein, “treatment,” “treating,” “palliating,” and “ameliorating” are used interchangeably. These terms refer to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. For prophylactic benefit, the complexes of the present invention may be administered to a subject, or a subject's cells or tissues, or those of another subject extracorporeally before re-administration, at risk of developing a particular disease, condition, or symptom, or to a subject reporting one or more of the physiological symptoms of a disease, condition, or symptom, even though the disease, condition, or symptom might not have yet been manifested.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 20” may mean from 18-22. Other meanings of “about” may be apparent from the context, such as rounding off; for example “about 1” may also mean from 0.5 to 1.4.

Discussion

According to a first embodiment, the present invention comprises a gRNA-ligand binding complex that comprises, consists essentially of, or consists of both a gRNA and a ligand binding moiety. This complex has the ability to retain association with a Type V Cas protein in the absence of a tracrRNA. Within the gRNA-ligand binding complex, the gRNA may be covalently bound directly to the ligand binding moiety or bound to the ligand binding moiety through a linker.

gRNA

The gRNA of the gRNA-ligand binding complex is single strand of nucleotides. The nucleotides may be entirely RNA or a combination of ribonucleotides and other nucleotides such as deoxyribonucleotides. Each nucleotide may be unmodified, or one or more nucleotides may be modified, e.g., with one of the following modifications: 2′-O-methyl, 2′ fluoro or 2′aminopurine. In some embodiments over one or more ranges of one to forty or two to twenty or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35, or 36 nucleotides, there are consecutively modified nucleotides or a modification pattern of every second, or every third or every fourth nucleotide being modified at its 2′ position with all other nucleotides being unmodified. Additionally or alternatively, between one or more pairs or every pair of consecutive nucleotides, there may be modified or unmodified internucleotide linkages.

In some embodiments, the gRNA is 35 to 60 or 36 to 60 nucleotides long or 40 to 55 nucleotides long. The gRNA has a sequence that may consist of, consist essentially of or comprise a crRNA sequence. Within the crRNA sequence are a Cas association region, which also may be referred to as the repeat region, that is 14 to 37 or 18 to 30 nucleotides long or 18 to 30 nucleotides long or 20 to 25 nucleotides long and a targeting region, which also may be referred to as a spacer region, that is 14 to 37 or 18 to 30 nucleotides long or 20 to 25 nucleotides long.

The targeting region contains the targeting sequence, which is a variable sequence that may be selected based on where one wishes for the Cas protein and/or effector to cause base editing. Thus, the targeting region may be designed to include a region that is complementary and capable of hybridization to a pre-selected target site of interest. For example, the region of complementarity between the targeting region and the corresponding target site sequence may be about 10, 11, 12, 13, 14, 15, 16, 17, 18, 19, 20, 21, 22, 23, 24, 25, or more than 25 consecutive nucleotides in length or it may be at least 80%, at least 85%, at least 90%, or at least 95% complementary to a region of DNA over 10, 11, 12, 13, 14, 15, 16, 17, 18, 19, 20, 21, 22, 23, 24, 25, or more than 25 consecutive nucleotides.

The Cas association region of the gRNA is designed such that it is capable of retaining association with an RNA binding domain of a Type V Cas protein in the absence of a tracrRNA. (Not all nucleotides within the Cas association need directly associate with the Cas protein.) Preferably, this association is possible under both naturally occurring conditions and under laboratory conditions in which the complex is to be used. In some embodiments, the gRNA has or encodes one of the following sequences:

SEQ ID NO: 1:
UUAAUUUCUACUCUUGUAGAUN₁₄₋₃₀;
or
SEQ ID NO: 2:
UGCUCGAUUAGUCGACACN₁₄₋₃₀;
or
SEQ ID NO: 3:
GGAGAGAUCUCAAACGAUUGCUCGAUUAGUCGAGACN₁₄₋₃₀;
or
SEQ ID NO: 4:
GUCGGAACGCUCAACGAUUGCCCCUCACGAGGGGACN₁₄₋₃₀
or
SEQ ID NO: 5:
ACCAAAACGACUAUUGAUUGCCCAGUACGCUGGGACN₁₄₋₃₀
or
SEQ ID NO: 6:
AUGGCAACAGACUCUCAUUGCGCGGUACGCCGCGACN₁₄₋₃₀
or
SEQ ID NO: 7:
GUCCCAACGAAUUGGGCAAUCAAAAAGGAUUGGAUCCN₁₄₋₃₀;
or
SEQ ID NO: 8:
CCUGCGAAACCUUUUGAUUGCUCAGUACGCUGAGACN₁₄₋₃₀;
or
SEQ ID NO: 9:
CUUUCAAGACUAAUAGAUUGCUCCUUACGAGGAGACN₁₄₋₃₀;
or
SEQ ID NO: 10:
GUAGAAGACCUCGCUGAUUGCUCGGUGCGCCGAGACN₁₄₋₃₀;
or
SEQ ID NO: 11:
UAAUUUCUACUCUUGUAGAUN₁₄₋₃₀;
or
SEQ ID NO: 12:
UAAUUUCUACUAAGUGUAGAUN₁₄₋₃₀;
or
SEQ ID NO: 67:
GCUUUCAAGACUAAUAGAUUGCUCCUUACGAGGAGACN₁₄₋₃₀;
or
SEQ ID NO: 68:
AGAAAUCCGUCUUUCAUUGACGGN₁₄₋₃₀;
or
SEQ ID NO: 69:
UAAUUUCUACUAAGUGUAGAUN₁₄₋₃₀;
or
SEQ ID NO: 70:
GCUUUCAAGACUAAUAGAUUGCUCCUUACGAGGAGACN₁₄₋₃₀;
or
SEQ ID NO: 71:
AGAAAUCCGUCUUUCAUUGACGGN₁₄₋₃₀.

The downstream portion of the crRNA sequence, shown as N₁₆₋₃₀ in SEQ ID NO: 1 to SEQ ID NO: 12 or SEQ ID NO: 67 to SEQ ID NO: 71, corresponds to the targeting region and the sequence upstream of that sequence corresponds to the Cas association region. N refers to any modified or unmodified nucleotide. In SEQ ID NO: 1 to 12, N₁₄₋₃₀ means that there can be 14, 15, 16, 17, 18, 19, 20, 21, 22, 23, 24, 25, 26, 27, 28, 29, or 30 nucleotides. In some embodiments, than there being 14 to 40 N nucleotides the are 14 to 37 nucleotides. In some embodiments, N is 16 to 30. In some embodiments, the Cas association region has a sequence that is at least 80%, at least 85%, at least 90%, at least 95% similar to or the same as the Cas association region (the region upstream of the Ns) of SEQ ID NO: 1 to SEQ ID NO: 12 or SEQ ID NO: 67 to SEQ ID NO: 71 or of a wildtype crRNA in a naturally occurring condition or endogenous to a naturally occurring or genetically modified organism.

Ligand Binding Moiety

The ligand binding moiety is an element that is capable of reversibly associating with a ligand by for example, forming non-covalent interactions. In some embodiments, the ligand binding moiety is an aptamer. The ligand binding moiety may be bound to the gRNA directly, e.g., through a covalent bond, or through a linker. The association of the ligand binding moiety with the gRNA, regardless of whether directly through a covalent bond or through a linker, may be at any of a number of locations. A ligand binding moiety is bound directly to a gRNA if it is bound to a nucleotide within the gRNA, e.g., to the backbone phosphate of a unit or to a sugar moiety or to a nitrogenous base of a nucleotide.

By way of non-limiting examples, the ligand binding moiety may be bound directly (through e.g., a covalent bond) to the 3′ end of the gRNA or to the 5′ end of the gRNA. Thus, the ligand binding moiety may be bound to the first or last nucleotide in the gRNA. When the ligand binding moiety is a nucleotide sequence and it is bound directly to the 5′ end or the 3′ end of the gRNA, it may be in the same 5′ to 3′ orientation as the gRNA. In these circumstances, there is a continuous strand of nucleotides that contains both the ligand binding moiety and the gRNA either
5′-[gRNA]-[ligand binding moiety]-3′ or
5′-[ligand binding moiety]-[gRNA]-3′.
   In other embodiments the ligand binding moiety may be directly attached to the gRNA in an opposite orientation and thus is either
5′-[gRNA]-3′-3′-[ligand binding moiety]-5′ or
3′-[ligand binding moiety]-5′-5′-[gRNA]-3′.
   When the ligand binding moiety is not a nucleotide sequence, it may be attached at either the 5′ or 3′ end of the gRNA to the phosphorous moiety, the sugar at e.g., the 2′, 3′ or 5′ position or the nitrogenous base.

The ligand binding moiety may also be attached to the gRNA at a position other than the 5′ end or the 3′. When the ligand binding moiety is a nucleotide sequence it may be inserted in the gRNA, and thus there may for example be a first section of the gRNA that is 5′ of the ligand binding moiety and a second section of the gRNA that is 3′ of the ligand binding moiety such that there is one oligonucleotide sequence:
5′-[first section of gRNA]-[ligand binding moiety]-[second section of gRNA]-3′.
   In some embodiments, the first section of the gRNA contains the entire Cas association region and the second section of the gRNA contains the entire targeting region. In other embodiments, the first section of the gRNA contains the entire Cas association region and a portion of the targeting region, while the second section of the gRNA contains the remainder of the targeting region. In other embodiments, the first section of the gRNA contains a portion of the Cas association region, while the second section of the gRNA contains the remainder of the Cas association and the entire targeting region. Relative to a gRNA that does not contain the ligand binding moiety, in the complex that contains the gRNA, and the ligand binding moiety inserted therein, there may be no deletion of nucleotides from either the Cas association region or the targeting region. Alternatively, there may be a deletion of one or more nucleotides (e.g., 1 to 10 nucleotides) at either or both sides of the location of insertion.

In some embodiments, when the ligand binding moiety is not a nucleotide sequence and it may be attached at either the 5′ or 3′ end of the gRNA to the phosphorous moiety, the sugar at e.g., the 2′, 3′ or 5′ position or the nitrogenous base. In other embodiments when the ligand binding moiety is not a nucleotide sequence, it may be bound to the gRNA at a location other than the 5′ or 3′ end of the gRNA, for example, it may be bound between two consecutive nucleotides as follows:
5′-[first section of gRNA]-[ligand binding moiety]-[second section of gRNA]-3′.

In some embodiments, one or more linkers binds the ligand binding moiety to the gRNA. In these embodiments, the linker and the ligand binding moiety each may independently comprise, consist essentially of, or consist of nucleotides. In some embodiments, each of the linker and the ligand binding moiety may independently comprise, consist essentially of, or consist of a moiety other than nucleotides. In some embodiments, one of the linker and the ligand binding moiety comprises, consists essentially of, or consists of a moiety other than nucleotides, while the other of the linker and the ligand binding moiety comprises, consists essentially of, or consists of nucleotides.

When the ligand binding moiety is a nucleotide sequence and it is bound through a linker that is also a nucleotide sequence to the 5′ end or the 3′ end of the gRNA, each of the gRNA, the linker and ligand binding moiety may be in the same 5′ to 3′ orientation. In these circumstances, there is a continuous strand of nucleotides that contains both the ligand binding moiety and the gRNA either
5′-[gRNA]-[linker]-[ligand binding moiety]-3′ or
5′-[ligand binding moiety]-[linker]-[gRNA]-3′.
   In other embodiments the ligand binding moiety and/or the linker can be directly attached to the gRNA in an opposite orientation and thus is
5′-[gRNA]-3′-3′-[linker]-5′-3′-[ligand binding moiety]-5′ or
5′-[gRNA]-3′-5′-[linker]-3′-3′-[ligand binding moiety]-5′ or
3′-[ligand binding moiety]-5′-3′-[linker]-5′-5′-[ligand binding moiety]-3′ or
3′-[ligand binding moiety]-5′-5′-[linker]-3′-5′-[gRNA]-3′.

The ligand binding moiety may also be attached to the gRNA through a linker or two linkers at a position other than the 5′ end or the 3′. When the ligand binding moiety and the linker(s) are nucleotide sequences, they may be inserted in the gRNA, and thus there may, for example, be a first section of the gRNA that is 5′ of the ligand binding moiety and a second section of the gRNA that is 3′ of the ligand binding moiety. There may also be one or two linker sequences.

When there is only one linker sequence it may be either 5′ or 3′ of the ligand binding moiety such that the complex is
5′-[first section of gRNA]-[linker]-[ligand binding moiety]-[second section of gRNA]-3′, or
5′-[first section of gRNA]-[ligand binding moiety]-[linker]-[second section of gRNA]-3′.

When there are two linker sequences, a first linker may be 5′ of the ligand binding moiety and the second linker may be 3′ of the ligand binding moiety such that the complex is
5′-[first section of gRNA]-[first linker]-[ligand binding moiety]-[second linker]-[second section of gRNA]-3′.

In some embodiments, each of the first section of gRNA, the first linker, the ligand binding moiety, the second linker, and the second section of gRNA are nucleotide sequences in the same orientation. In other embodiments, one or more of the first linker, ligand binding moiety and the second linker are in the opposite orientation to that of the first section of gRNA and the second section of gRNA, which are in the same orientation.

When the ligand binding moiety is between the first section of the gRNA and the second section of the gRNA (and if one or two linkers are present they are also between the first section of the gRNA and the second section of the gRNA), in some embodiments, the first section of the gRNA contains the entire Cas association region and the second section of the gRNA contains the entire targeting region. In other embodiments, the first section of the gRNA contains the entire Cas association region and a portion of the targeting region, while the second section of the gRNA contains the remainder of the targeting region. In other embodiments, the first section of the gRNA contains a portion of the Cas association region, while the second section of the gRNA contains the remainder of the Cas association and the entire targeting region. Relative to a gRNA that does not contain the ligand binding moiety, in a complex that contains the gRNA and the ligand binding moiety inserted, there may be no deletion of nucleotides from either the Cas association region or the targeting region. Alternatively, there may be a deletion of one or more nucleotide (e.g., 1 to 10 nucleotides) at the location of insertion.

When there are two linkers present, they may be of sufficient complementary such that they can hybridize to each under. For example, each linker may be 1 to 20 nucleotides long and the linkers may be at least 80%, at least 85%, at least 90%, at least 95% at least 98% or 100% complementary and have no bulges or one or more bulges.

When the linker is not a nucleotide sequence, it may be bound to the 5′ most nucleotide within the gRNA, the 3′ most nucleotides within the gRNA or a nucleotide other than the 5′ most nucleotide or the 3′ most nucleotide within the gRNA. Further, a linker that is not a nucleotide or oligonucleotide may be attached at any position of a sugar or nitrogenous base or be attached to or replace an internucleotide linkage. Additionally, in some embodiments, there are two non-nucleotide linkers or one nucleotide linker and one non-nucleotide linker.

In some embodiments, the gRNA forms a loop and the ligand binding moiety or the linker if present is bound to the loop. When bound to the loop of the gRNA, either directly or through a linker, the bonding may, for example, be at the first nucleotide in the loop, the second nucleotide in the loop, the third nucleotide in the loop, the fourth nucleotide in the loop, the center nucleotide in the loop if the loop has an odd number of nucleotides or one of the two center most nucleotides in the loop if the loop has an even number of nucleotides, or the last nucleotide in the loop. Any one or more of the aforementioned nucleotides and/or the 5′ and/or 3′ internucleotide linkage corresponding to them may be modified. These modifications may, for example, occur where the ligand binding moiety is bound to the gRNA (directly or through a linker) or only at locations other than where the ligand binding moiety is bound to the gRNA (directly or through a linker). For example, the ligand binding moiety can be attached to a 2′ position of a sugar or attached to a nitrogenous base in the gRNA oligonucleotide sequence.

In some embodiments, the ligand binding moiety comprises, consists essentially of, or consists of an oligonucleotide sequence that is unmodified or comprises one or more modified nucleotides. For example, the ligand binding moiety may be 10 to 50 or 18 to 50 nucleotides long. In one embodiment, the ligand binding moiety comprises, consists essentially of, or consists of SEQ ID NO: 13 (ACAUGAGGAUCACCCAUGU) or a sequence that is substantially similar to SEQ ID NO: 13. In some embodiments the ligand binding moiety forms a stem-loop structure. If there is no linker present, the ligand binding moiety may appear as an extension of the gRNA sequence immediately 5′ or 3′ of the gRNA or as an insert in the gRNA.

In some embodiments, the ligand binding moiety comprises, consists essentially of, or consists of biotin or streptavidin.

In some embodiments the ligand binding moiety can attach covalently or non-covalently.

In some embodiments, the ligand binding moiety is selected from the group consisting of moieties that associate with the following ligands: MS2 coat protein (MCP), Ku, PP7 coat protein (PCP), Com RNA binding protein or the binding domain thereof, SfMu, Sm7, Tat, Glutathione S-transferase (GST), CSY4, Qbeta, COM, pumilio, Anti-His Tag (6H7), SNAP-Tag, lambdaN22, a lectin (in which case ligand binding moiety may be carbohydrate or glycan or oligosaccharide), and PDGF beta-chain. In some embodiments, the ligand binding moiety is an aptamer that comprises deoxyribonucleotides, ribonucleotides or a combination of both. Therefore, as non-limiting examples, one may use DNA aptamers, RNA aptamers, DNA aptamers with modified nucleosides in the backbones, RNA aptamers with modified nucleosides in the backbones and combinations thereof.

In some embodiments, a naturally occurring MS2 aptamer is used as the ligand binding moiety. In other embodiments, one uses an MS2 C-5 mutant or an MS2 F-5 mutant or a modified MS2, e.g., MS2 in which there is one or more, e.g., 1, 2, 3, 4, 5, 6, 7, 8, 9, 10, 11, or 12, modified nucleotides such as an amino purine, at position 10, wherein position 10 is the tenth nucleotide from the 5′ end of an aptamer. The 2-amino purine may, for example, be 2-amino purine is 2′ deoxy-2-aminopurine or 2′ ribose 2-aminopurine. The modification at any one position or may be in addition to a modification at another position or to the exclusion of a modification at any or all of the other positions.

In some embodiments, the ligand binding moiety is an aptamer that comprises a 5′ modified nucleotide, wherein the 5′ modified nucleotide comprises at least one of a 2′ modification, a 5′ PO4 group, or a modification of the nitrogenous base.

In some embodiments, the ligand binding moiety is an aptamer that is or comprises one part of an aptamer-ligand pair, and as discussed below, and the effector is linked to or comprises the other part of the aptamer-ligand pair. For example, the aptamer may comprise a MS2 operator motif that specifically binds to an MS2 coat protein, MCP. As persons of ordinary skill in the art will appreciate alternatively, the aptamer can comprise the MCP moiety (or other ligand) in which case the effector would comprise or be linked to the MS2 operator motif (or other corresponding ligand binding moiety).

Linkers

A linker, when present, may be a species that connects the ligand binding moiety to the gRNA. It may be attached to each of the ligand binding moiety and the gRNA at one location or it may be attached to either or both of the gRNA and the ligand binding moiety at a plurality of locations. Attachments at a plurality of locations may allow for greater control in three dimensional space of the ligand binding moiety and in turn the effector to be used.

By way of non-limiting examples, a linker may attach to the gRNA at one location and to the ligand binding moiety at two or more locations; or the linker may attach to the ligand binding moiety at one location and to the gRNA at two or more locations. When the linker is attached to the gRNA at two or more locations, the linker may be attached to the gRNA exclusively in the targeting region or exclusively in the Cas association region or in both regions.

In some embodiments, the linker comprises, consists essentially of, or consists of an oligonucleotide sequence and optionally the linker comprises at least one or a plurality of 2′ modifications, e.g., all nucleotides are 2′ modified nucleotides within the linker. The nucleotide sequence may be random or intentionally designed not to be undesirably complementary to sequence within the aptamer, the gRNA or the target site of the DNA. In some embodiments in which there are two linkers, the two linkers flank the ligand binding moiety.

In some embodiments, the linker comprises, consists essentially of, or consists of at least one phosphorothioate linkage.

In some embodiments, the linker comprises, consists essentially of, or consists of a levulinyl moiety.

In some embodiments, the linker comprises, consists essentially of, or consists of an ethylene glycol moiety.

In some embodiments, the linker comprises or is selected from the group consisting of 18S, 9S or C3.

In some embodiments, the linker is a nucleotide sequence that is one to sixty or one to twenty-four or two to twenty or five to fifteen nucleotides long. Additionally, in some embodiments, the linker is GC rich, e.g., having at least 50%, at least 60%, at least 70%, at least 80% or at least 90% GC nucleotides. When a linker comprises nucleotides, it may, for example, be single stranded or double stranded or partially single stranded and partially double stranded. Additionally, when a linker is an oligonucleotide, the linker may be exclusively RNA, exclusively DNA or a combination thereof.

In some embodiments, the linker is a nucleotide sequence that is upstream or downstream of the ligand binding moiety. When the linker is upstream of a ligand binding moiety and the gRNA is upstream of the linker, there may be another sequence that is complementary to the linker that is downstream of the ligand binding moiety. Similarly, when the linker is downstream of a ligand binding moiety and the gRNA is downstream of the linker, there may be another sequence that is complementary to the linker that is upstream of the ligand binding moiety. As persons of ordinary skill in the art will recognize, complementarity is determined when the oligonucleotide self-folds and the strands align with each relevant section in a 5′ to 3′ direction.

Thus, in some embodiments, the ligand binding moiety, e.g., MS2 has an upstream sequence that is 1 to 12 nucleotides long and a downstream sequence that is 1 to 12 nucleotides long, wherein the upstream and downstream sequences immediately flank the ligand binding moiety (i.e., there are no other nucleotides between the ligand binding moiety and each of the upstream and downstream sequences) and the upstream sequence is complementary to the downstream sequence. In some embodiments, each of the upstream sequence and the downstream sequence is 1 nucleotide long, 2 nucleotides long, 3 nucleotides long, 4 nucleotides long, 5 nucleotides long, 6 nucleotides long, 7 nucleotides long, 8 nucleotides long, 9 nucleotides long, 10 nucleotides long, 11 nucleotides long, or 12 nucleotides long. In one embodiment each of the upstream sequence and the downstream sequence comprises or is GC. When there are both upstream and downstream sequences, they may also be referred to as extension sequences.

In some embodiments, both the upstream and downstream sequence is two nucleotides long or three nucleotides long or four nucleotides long. In some embodiments, one of the upstream and downstream sequence is two nucleotides long and the other of the upstream sequence and the downstream sequence is four nucleotides long. In some embodiments, one or both of the linker sequences is or encodes GC or GCGC. In some embodiments, one of the upstream or downstream linker is GC and the other of the upstream or downstream linker is GCGC.

Modifications

In some embodiments, at least one of the gRNA or the ligand binding moiety is modified, or if a linker is present, at least one of the gRNA, the ligand binding moiety or the linker is modified. The modification refers to the introduction of a moiety or species that does not occur under naturally occurring conditions. Modifications may be used to increase one or both of stability and specificity. In some embodiments, stability is improved with respect to resistance to one or both of the active domain of the Cas protein (e.g., RuvC domain) and the active domain of one or more other enzymes within the system into which a complex of the present invention is introduced, including but not limited to any effector. The resistance may, in some embodiments, be caused by steric hindrance. In some embodiments, the modification(s) is/are located within and/or between one or more if not all of the nucleotides within the targeting region.

Specificity is improved when a modification reduces the likelihood of an off-target effect and/or increases the likelihood that a base editing complex of the present invention will reach its target site. Nucleotides may be modified at the ribose, phosphate linkage, and/or base moiety. For example, a phosphorothioate backbone may be used, at one, a plurality or all positions within the gRNA, the targeting region or the Cas association region and/or the ligand binding moiety and/or linker if present.

In some embodiments, the modification is the presence of one or more 2′ modified nucleotides (e.g., 2′-O-methyl or 2′-fluoro) and/or the presence of a phosphorothioate internucleotide linkage or the introduction of a 5′-PO₄ group of the gRNA and/or ligand binding moiety.

When more than one modification is present, the modifications may, for example, all be in the targeting region; all be in the Cas association region; all be in the ligand binding moiety; all be in the linker if present; be in both the targeting region and the Cas association region; be in both the Cas association region and the ligand binding moiety; be in both the Cas association region and the linker if present; be in both the targeting region and the ligand binding moiety; be in both the targeting region and the linker if present; be in both the ligand binding moiety and the linker if present; be in all three of the Cas association region, the targeting region and the ligand binding moiety; be in the Cas association region, the targeting region and the linker if present; be in the Cas association region, the ligand binding moiety and the linker if present; be in the targeting region, the ligand binding moiety and the linker if present; or be in each of the Cas association region, the targeting region, the ligand binding moiety and the linker if present.

In some embodiments, there are one to sixty or one to thirty or one to ten or ten to twenty or twenty to thirty or thirty to forty or forty to fifty or fifty to sixty 2′ modifications. By way of non-limiting examples, the set of 2′ modifications may be located in the targeting region; the set of 2′ modifications may be located in the ligand binding moiety if the ligand binding moiety is or comprises an oligonucleotide sequence; or the set of 2′ modifications may be located in the Cas association region. The modifications may be on consecutive nucleotides or there may be one or more pairs of unmodified nucleotides between modified nucleotides in regular or irregular patterns. By way of a further non-limiting example, within a gRNA any one or more of positions 1, 2, 3, 4, 5, 6, 7, 8, 9, 10, 11, 12, 13, 14, 15, 16, 17, or 18 comprises a 2′-O-alkyl group, wherein the positions are measure from the 5′ end or the 3′ end of the gRNA.

In some embodiments, in addition to or in the absence of 2′ modified nucleotides there are modified internucleotide linkages such as a phosphorothioate linkage. Examples of modifications to the backbones of the gRNA, the aptamer (in an oligonucleotide), and the linker (if present and an oligonucleotide), include but are not limited to phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i. e., a single inverted nucleoside residue that may be abasic (the nucleobase is missing or has a hydroxyl group in place thereof). Various salts (such as, for example, potassium or sodium), mixed salts and free acid forms of the aforementioned internucleotide linkages are also included within the scope of the present invention.

Also within the scope of the present invention is the use of polynucleotide backbones that do not include a phosphorus atom therein and instead have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These modifications include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In some embodiments, one or more of the parts of a complex has one to sixty or one to twenty or one to ten or ten to twenty or twenty to thirty or thirty to forty or forty to fifty or fifty to sixty phosphorothioate linkages. These phosphorothioate linkages may: all be in the Cas association region; all be in the ligand binding moiety; all be in the linker; be in both the targeting region and the Cas association region; be in both the Cas association region and the ligand binding moiety; be in both the Cas association region and the linker if present; be in both the targeting region and the ligand binding moiety; be in both the targeting region and the linker if present; be in both the ligand binding moiety and the linker if present; be in all three of the Cas association region, the targeting region and the ligand binding moiety; be in the Cas association region, the targeting region and the linker if present; be in the Cas association region, the ligand binding moiety and the linker if present; be in the targeting region, the ligand binding moiety and the linker if present; or be in each of the Cas association region, the targeting region, the ligand binding moiety and the linker if present.

Any nucleotide within a complex of the present invention may include one or more substituted sugar moieties. These nucleotides may comprise a sugar substituent group selected from: OH; H; F; O—, S—, or N-alkyl; O—, S—, or N-alkenyl; O—, S— or N-alkynyl; or O-alkyl-Co-alkyl, wherein the alkyl, alkenyl and alkynyl may be substituted or unsubstituted C1 to C10 alkyl or C2 to C10 alkenyl and alkynyl. Particularly suitable are O((CH₂)nO)mCH₃, O(CH₂)nOCH₃, O(CH2)nNH₂, O(CH₂)nCH₃, O(CH₂)nONH₂, and O(CH₂)nON((CH₂)nCH₃)₂, where n and m are from 1 to about 10. Other suitable nucleotides comprise a sugar substituent group selected from: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. By way of a non-limiting example, a suitable modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) or another alkoxyalkoxy group. A further suitable modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—OCH₂CH₂CH2NH₂), allyl (—CH₂—CH═CH₂), —O-allyl CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide.

Any nucleotide within a complex of the present invention may also include nucleobase (often referred to in the art simply as “base”) modifications or substitutions. Modified nucleobases include, but are not limited to other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include, but are not limited to tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one) and 5-methoxy uracil.

Heterocyclic base moieties may also include, but are not limited to, those in which the purine or pyrimidine base is replaced with other heterocycles, for example, 7-deaza-adenine, 7-deazaguanosine, 2-aminopyridine and 2-pyridone. Examples of other nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound: 5-substituted pyrimidines, 6-azapyrimidines and N-2, N-6 and O-6 substituted purines, including 2-aminopropyladenine, 5-propynyluracil and 5-propynylcytosine. Additionally, 5-methylcytosine substitutions may be advantageous when combined with 2′-O-methoxyethyl sugar modifications.

In some embodiments, there are two ligand binding moieties associated with a gRNA: a first ligand binding moiety and a second ligand binding moiety. Optionally, when there are two ligand binding moieties, there may be two linkers: a first linker and a second linker, wherein the first ligand binding moiety is attached to the first linker and the second ligand binding moiety is attached to the second linker. In these embodiments, the first linker and the second linker may each be attached to the Cas association region; or the first linker and the second linker may each be attached to the targeting region; or one of the first linker and the second linker may be attached to the Cas association region and the other of the first linker and the second linker may be attached to the targeting region.

Base Editing Complexes

According to another embodiment of the present invention, there is a base editing complex. The base editing complex comprises, consists essentially of, or consists of a gRNA-ligand binding complex of the present invention; and a Type V Cas protein, wherein the Cas association region of the gRNA-ligand binding complex is associated with the Type V Cas protein. Thus, the gRNA is capable of associating the gRNA with the Cas protein and delivering the Cas protein to the target nucleic acid without the need of a tracrRNA.

An example of a base editing complex of the presentation invention is shown in FIG. 12. A gRNA 310 is associated with a Cas protein 340. The ligand binding moiety 320 is attached to the 5′ end of the gRNA. The effector 350, which may, for example, be a deaminase has been recruited by RNA-ligand binding interaction with the ligand 330 at 360.

Type V Cas Protein

In general, a Cas protein includes at least one RNA binding domain. The RNA binding domain interacts with the gRNA at the Cas association region. The Type V Cas protein that is of use in the present invention is one with which the gRNA-ligand binding complex can associate without there being a tracrRNA present. In some embodiments, the Type V Cas protein is an endonuclese that contains a RuvC domain. This RuvC domain may be mutated such that the endonuclease activity is deactivated. In some embodiments, the protein is a nickase that contains an active or deactivated RuvC domain.

Examples of Type V Cas proteins that may be of use in connection with the present invention include, but are not limited to, Cas12a, MAD7 (an engineered variant of ErCas12a), Cas12h, Cas12i, and Cas12j (CasPhi, also known as Casϕ) in active or deactivated form.

In some embodiments, the Type V Cas proteins comprise a fusion protein having: (a) an active, partially deactivated or deactivated Type V Cas protein; and (b) a uracil DNA glycosylase (UNG) inhibitor peptide (UGI). The UGI peptide can be fused directly to the Type V Cas protein or through a linker peptide comprised of 1 to 100 hundred amino acid residues. In some embodiments, the UGI comprises the wild type UGI sequence from the Bacillus phage PBS2

(https://www.ncbi.nlm nih.gov/protein/P14739):

### MTNLSDIIEKETGKQLVIQESILMLPEEVEEVIGNKPESDILVHTAYDESTDENV

MLLTSDAPEYKPWALVIQDSNGENKIKML (SEQ ID NO: 140). In some embodiments, the UGI comprises variants of SEQ ID NO: 140 that comprises a fragment of the wild type UGI peptide or a homologous amino acid sequence to SEQ ID NO: 28. In some embodiments, the UGI fragment of homologous sequence comprises at least 60%, at least 65%, at least 70%, at least 75%, at least 80%, at least 85%, at least 90%, at least 95%, at least 97%, at least 99%, or at least 99.5% homology to the wild type UGI peptide sequence (SEQ ID NO: 140).

In some embodiments, the active or deactivated Type V Cas protein comprises a fusion with two or more UGI peptides or variants. The UGI peptides, or variants of the UGI peptide, can be connected directly to another UGI peptide or Type V Cas protein or via a linker of 1 to 100 amino acid residues to another UGI peptide or Type V Cas protein.

The Cas protein or Cas protein fusion may be provided in purified or isolated form or can be part of a composition or complex. Preferably, when in a composition, the protein is first purified to some extent, more preferably to a high level of purity (e.g., about 80%, 90%, 95%, or 99% or higher). Compositions in which the complexes and components of the present invention may be stored and transported may be any type of composition desired, e.g., aqueous compositions suitable for use as, or inclusion in, a composition for RNA-guided targeting.

Effectors

The base editing complexes of the present invention may contain an effector that is attached to a ligand. The ligand is capable of reversibly or irreversibly associating with the ligand binding moiety. Thus, the ligand binding moiety recruits an effector, e.g. base editing enzyme that is fused to or otherwise associated with the ligand, because the ligand binding moiety is capable of retaining association with the ligand. This design may be particularly advantageous because it provides a modular design in which the nucleic acid sequence targeting function of the gRNA and effector function reside in different molecules. For example, to introduce modifications serially at the same site, one may use different effectors that are associated with the same ligand. Conversely, to introduce the same modifications at different sites, one may use the same ligand binding moiety with different gRNAs while using the same effector-ligand. Thus, this design allows one to multiplex a system without an undesirable burden of fusing effectors to either gRNAs or Cas proteins.

Examples of effectors that may be of use in connection with the present invention are deaminases such as those that have cytidine deamination or adenine deamination activity, as well as transcriptional regulators, repair enzymes, epigenetic modifiers, histone acetylases, deacetylases, methylases (of histones ad nucleotides), and demethylases (of histones and nucleotides). In some embodiments, the effector is selected from the group consisting of AID, CDA, APOBEC1, APOBEC3A, APOBEC3B, APOBEC3C, APOBEC3D, APOBEC3F, ADA, ADAR and tRNA adenosine deaminase. Examples of effectors and the types of genetic change that they case are provided in table 1.

TABLE 1
Examples of effector proteins

| | | Effector protein |
|---|---|---|
| Enzyme type | Genetic change | abbreviated |
| Cytidine deaminase | C→U/T | AID |
| | | APOBEC1 |
| | | APOBEC3A |
| | | APOBEC3B |
| | | APOBEC3C |
| | | APOBEC3D |
| | | APOBEC3F |
| | | APOBEC3G |
| | | APOBEC3H |
| Adenosine | A→I/G | ADA |
| deaminase | | ADAR1 |
| | | TadA |
| | | TADA |
| | | TAD3 |
| DNA Methyl | C→Met-C | ADAR2 |
| transferase | | ADAR3 |
| | | Dnmt1 |
| Demethylase | Met-C→C | Dnmt3a |
| Cytidine | 5mC → 5hmC | TET1 |
| demethylase | | |
| Cytidine | 5mC → 5hmC | TET2 |
| demethylase | 5hmC → 5fC/5caC | |
| Glycosylase | 5fc/5caC → C | TDG |

Effector protein full names:
AID: activation induced cytidine deaminase, a.k.a AICDA
APOBEC1: apolipoprotein B mRNA editing enzyme, catalytic polypeptide-like 1.
APOBEC3A: apolipoprotein B mRNA editing enzyme, catalytic polypeptide-like 3A
APOBEC3B: apolipoprotein B mRNA editing enzyme, catalytic polypeptide-like 3B
APOBEC3C: apolipoprotein B mRNA editing enzyme, catalytic polypeptide-like 3C
APOBEC3D: apolipoprotein B mRNA editing enzyme, catalytic polypeptide-like 3D
APOBEC3F: apolipoprotein B mRNA editing enzyme, catalytic polypeptide-like 3F
APOBEC3G: apolipoprotein B mRNA editing enzyme, catalytic polypeptide-like 3G
APOBEC3H: apolipoprotein B mRNA editing enzyme, catalytic polypeptide-like 3H
ADA: adenosine deaminase
ADAR1: adenosine deaminase acting on RNA 1
ADAR2: adenosine deaminase acting on RNA 2
ADAR3: adenosine deaminase acting on RNA 3
Dnmt1: DNA (cytosine-5-)-methyltransferase 1
Dnmt3a: DNA (cytosine-5-)-methyltransferase 3 alpha
TadA: tRNA-specific adenosine deaminase
TADA: tRNA(adenine(34)) deaminase, chloroplastic
TAD3: tRNA-specific adenosine deaminase TAD3
TET1: Methylcytosine dioxygenase TET1
TET2: Methylcytosine dioxygenase TET2
TDG: G/T mismatch-specific thymine DNA glycosylase

In some embodiments, the base editing complex comprises two or more effectors. When there are two effectors they may be referred to as: a first effector and a second effector. Each effector may be attached to a different ligand binding moiety through a different ligand. Alternatively, when there are two effectors present, one is attached to a ligand and associated with the gRNA through the ligand binding moiety and another is attached directly to the Cas protein. Examples of sequences of deaminases that may be incorporated into the present invention include but are not limited to:

hA3A (SEQ ID NO: 149):
ATGGAGGCATCTCCAGCATCCGGTCCAAGGCATCTCATGGATCCCC
ATATCTTCACCTCCAATTTTAATAACGGAATCGGGCGCCACAAGAC
ATACTTGTGCTATGAGGTGGAACGACTGGACAACGGTACCTCCGTG
AAAATGGACCAACATCGCGGATTTCTGCATAATCAGGCTAAAAACC
TTCTGTGTGGATTTTATGGGAGACACGCTGAGCTGAGATTTCTTGA
CCTGGTCCCGAGCTTACAGCTGGACCCAGCCCAAATCTATCGCGTA
ACTTGGTTCATCAGCTGGAGCCCCTGCTTTTCCGCCGGGTGCGCTG
GAGAAGTGCGGGCGTTCCTGCAGGAAAACACCCACGTCAGACTGA
GGATTTTTGCAGCACGCATCTACGACTATGATTATCTTTACAAGGA
GGCATTACAGATGTTGCGCGATGCCGGAGCCCAAGTAAGCATTATG
ACTTATGATGAGTTCAAACACTGTTGGGACACCTTTGTAGACCACC
AGGGCTGCCCCTTTCAGCCTTGGGATGGGCTCGACGAGCACAGCCA
GGCACTCAGCGGACGCCTCCGCGCTATCCTCCAGAACCAGGGTAAC
ratAPO (SEQ ID NO: 33):
ATGTCCTCAGAGACTGGGCCTGTCGCCGTCGATCCAACCCTGCGCC
GCCGGATTGAACCTCACGAGTTTGAAGTGTTCTTTGACCCCCGGGA
GCTGAGAAAGGAGACATGCCTGCTGTACGAGATCAACTGGGGAGG
CAGGCACTCCATCTGGAGGCACACCTCTCAGAACACAAATAAGCA
CGTGGAGGTGAACTTCATCGAGAAGTTTACCACAGAGCGGTACTTC
TGCCCCAATACCAGATGTAGCATCACATGGTTTCTGAGCTGGTCCC
CTTGCGGAGAGTGTAGCAGGGCCATCACCGAGTTCCTGTCCAGATA
TCCACACGTGACACTGTTTATCTACATCGCCAGGCTGTATCACCAC
GCAGACCCAAGGAATAGGCAGGGCCTGCGCGATCTGATCAGCTCC
GGCGTGACCATCCAGATCATGACAGAGCAGGAGTCCGGCTACTGCT
GGCGGAACTTCGTGAATTATTCTCCTAGCAACGAGGCCCACTGGCC
TAGGTACCCACACCTGTGGGTGCGCCTGTACGTGCTGGAGCTGTAT
TGCATCATCCTGGGCCTGCCCCCTTGTCTGAATATCCTGCGGAGAA
AGCAGCCCCAGCTGACCTTCTTTACAATCGCCCTGCAGTCTTGTCAC
TATCAGAGGCTGCCACCCCACATCCTGTGGGCCACAGGCCTGAAG
GgAID sequence (SEQ ID NO: 34):
ATGGATTCTCTGCTGATGAAGAGGAAGCTGTTTCTGTACAATTTTA
AGAATCTGAGGTGGGCCAAGGGCAGAAGGGAGACCTATCTGTGCT
ACGTGGTGAAGAGAAGGGACAGCGCCACCAGCTGCAGCCTCGATT
TCGGCTATCTGAGGAACAAGATGGGCTGTCACGTGGAGGTGCTGTT
TCTGAGATACATCTCCGCTTGGGATCTGGATCCCGGCAGATGCTAT
AGAATCACATGGTTCACCAGCTGGAGCCCTTGTTACGACTGTGCTA
GACATGTGGCCGACTTTCTGAGGGCCTATCCCAATCTGACACTGAG
AATCTTCACCGCTAGACTGTACTTCTGCGAGGACAGAAAGGCTGAG
CCCGAGGGACTGAGAAGGCTGCACAGAGCCGGCGCCCAGATCGCC
ATCATGACCTTTAAGGACTTTTTCTATTGCTGGAACACCTTCGTGGA
GAATAGAGAGAAGACCTTCAAGGCTTGGGAGGGACTGCACGAGAA
CTCCGTGCATCTGTCTAGAAAGCTGAGGAGAATTCTGCTGCCTCTG
TATGAGGTGGACGATCTGAGAGATGCCTTCAAGACCCTCGGACTG
hAID (SEQ ID NO 141):
ATGGATAGCCTGCTGATGAACCGGAGAAAGTTCCTGTATCAGTTTA
AGAATGTGCGCTGGGCAAAGGGCAGGCGCGAGACCTACCTGTGCT
ATGTGGTGAAGCGGAGAGATTCCGCCACATCCTTCTCTCTGGACTT
TGGCTACCTGCGGAACAAGAATGGCTGCCACGTGGAGCTGCTGTTC
CTGAGATACATCTCTGACTGGGATCTGGACCCAGGCAGGTGTTATC
GCGTGACCTGGTTCACAAGCTGGTCCCCCTGCTACGATTGTGCAAG
GCACGTGGCAGACTTTCTGAGGGGAAACCCAAATCTGTCCCTGCGG
ATCTTCACCGCCAGACTGTATTTTTGCGAGGATAGGAAGGCAGAGC
CAGAGGGACTGAGGCGCCTGCACAGGGCCGGCGTGCAGATCGCCA
TCATGACCTTCAAGGACTACTTTTATTGTTGGAACACCTTCGTGGAG
AATCACGAGCGGACCTTCAAGGCCTGGGAGGGACTGCACGAGAAC
TCCGTGCGGCTGTCTAGACAGCTGCGGAGAATCCTGCTGCCTCTGT
ACGAGGTGGACGATCTGAGGGATGCCTTCCGCACCCTGGGACTG.

Ligands

As noted above, the effector is bound to a ligand, e.g., by one or more covalent bonds. A non-exhaustive list of examples of ligand binding moiety-ligand pairs that may be used in various embodiments of the present invention is provided in Table 2. Both unmodified and chemically modified versions or the ligand binding moieties and ligands are within the scope of the present invention.

TABLE 2

| Ligand Binding Moieties | Ligands |
|---|---|
| Telomerase Ku binding motif | Ku |
| Telomerase Sm7 binding motif | Sm7 |
| MS2 phage operator stem-loop | MS2 Coat Protein (MCP) |
| PP7 phage operator stem-loop | PP7 coat protein (PCP) |
| SfMu phage Com stem-loop | Com RNA binding protein |
| Non-natural RNA aptamer | Corresponding aptamer ligand |
| Biotin | Streptavidin |
| Oligosaccharide | Lectin |
| Benzylguanine or benzylcytosine | SNAP/CLIP tag |
| 6x-His binding motif | 6x-His tag |
| PDGFbeta chain binding motif | PDGF B-chain |
| GST binding motif | GST protein |
| Tat binding motif | BIV Tat protein |
| Tat binding motif | HIV Tat protein |
| Pumilio binding motif | PUM-HD domain |
| BoxB binding motif | Lambda N22plus |
| Csy4 binding motif | Csy4[H29A] |

Some of the sequences for the above binding pairs are listed below.

1. Telomerase Ku binding motif/Ku heterodimer
a. Ku binding hairpin
(SEQ ID No: 14)
5′-UUCUUGUCGUACUUAUAGAUCGCUACGUUAUUUCAAUUUUGAAAAUC
UGAGUCCUGGGAGUGCGGA-3′
b. Ku heterodimer
(SEQ ID NO: 15)
MSGWESYYKTEGDEEAEEEQEENLEASGDYKYSGRDSLIFLVDASKAMFE
SQSEDELTPFDMSIQCIQSVYISKIISSDRDLLAVVFYGTEKDKNSVNFK
NIYVLQELDNPGAKRILELDQFKGQQGQKRFQDMMGHGSDYSLSEVLWVC
ANLFSDVQFKMSHKRIMLFTNEDNPHGNDSAKASRARTKAGDLRDTGIFL
DLMHLKKPGGFDISLFYRDIISIAEDEDLRVHFEESSKLEDLLRKVRAKE
TRKRALSRLKLKLNKDIVISVGIYNLVQKALKPPPIKLYRETNEPVKTKT
RTFNTSTGGLLLPSDTKRSQIYGSRQIILEKEETEELKRFDDPGLMLMGF
KPLVLLKKHHYLRPSLFVYPEESLVIGSSTLFSALLIKCLEKEVAALCRY
TPRRNIPPYFVALVPQEEELDDQKIQVTPPGFQLVFLPFADDKRKMPFTE
KIMATPEQVGKMKAIVEKLRFTYRSDSFENPVLQQHFRNLEALALDLMEP
EQAVDLTLPKVEAMNKRLGSLVDEFKELVYPPDYNPEGKVTKRKHDNEGS
GSKRPKVEYSEEELKTHISKGTLGKFTVPMLKEACRAYGLKSGLKKQELL
EALTKHFQD
(SEQ ID No: 16)
MVRSGNKAAVVLCMDVGFTMSNSIPGIESPFEQAKKVITMFVQRQVFAEN
KDEIALVLFGTDGTDNPLSGGDQYQNITVHRHLMLPDFDLLEDIESKIQP
GSQQADELDALIVSMDVIQHETIGKKFEKRHIEIFTDLSSRFSKSQLDII
IHSLKKCDISERHSIHWPCRLTIGSNLSIRIAAYKSILQERVKKTWTVVD
AKTLKKEDIQKETVYCLNDDDETEVLKEDIIQGFRYGSDIVPFSKVDEEQ
MKYKSEGKCFSVLGFCKSSQVQRRFFMGNQVLKVFAARDDEAAAVALSSL
IHALDDLDMVAIVRYAYDKRANPQVGVAFPHIKHNYECLVYVQLPFMEDL
RQYMFSSLKNSKKYAPTEAQLNAVDALIDSMSLAKKDEKTDTLEDLFPTT
KIPNPRFQRLFQCLLHRALHPREPLPPIQQHIWNMLNPPAEVTTKSQIPL
SKIKTLFPLIEAKKKDQVTAQEIFQDNHEDGPTAK
2. Telomerase Sm7 binding motif/Sm7 homoheptamer
a. Sm consensus site (single stranded)
(SEQ ID No: 17)
5′-AAUUUUUGGA-3′
b. Monomeric Sm-like protein (archaea)
(SEQ ID No: 18)
GSVIDVSSQRVNVQRPLDALGNSLNSPVIIKLKGDREFRGVLKSFDLHMN
LVLNDAEELEDGEVTRRLGTVLIRGDNIVYISP
3. MS2 phage operator stem loop/MS2 coat protein
a. MS2 phage operator stem loop
(SEQ ID No: 19)
5′-ACAUGAGGAUCACCCAUGU-3′
b. MS2 coat protein
(SEQ ID No: 20)
MASNFTQFVLVDNGGTGDVTVAPSNFANGIAEWISSNSRSQAYKVTCSVR
QSSAQNRKYTIKVEVPKGAWRSYLNMELTIPIFATNSDCELIVKAMQGLL
KDGNPIPSAIAANSGIY
4. PP7 phage operator stem loop/PP7 coat protein
a. PP7 phage operator stem loop
(SEQ ID No: 21)
5′-AUAAGGAGUUUAUAUGGAAACCCUUA-3′
b. PP7 coat protein (PCP)
(SEQ ID No: 22)
MSKTIVLSVGEATRTLTEIQSTADRQIFEEKVGPLVGRLRLTASLRONGA
KTAYRVNLKLDQADVVDCSTSVCGELPKVRYTQVWSHDVTIVANSTEASR
KSLYDLTKSLVATSQVEDLVVNLVPLGR
5. SfMu Com stem loop/SfMu Com binding protein
a. SfMu Com stem loop
(SEQ ID No: 23)
5′-CUGAAUGCCUGCGAGCAUC-3′
b. SfMu Com binding protein
(SEQ ID No: 24)
MKSIRCKNCNKLLFKADSFDHIEIRCPRCKRHIIMLNACEHPTEKHCGKR
EKITHSDETVRY
6. BoxB aptamer/lambda N22plus
a. BoxB aptamer
(SEQ ID No: 25)
5′-GCCCUGAAGAAGGGC-3′
b. Lambda N22plus protein
(SEQ ID No: 26)
MNARTRRRERRAEKQAQWKAAN
7. Csy4 binding stem loop/Csy4[H29A]
a. Csy4 binding motif
(SEQ ID No: 27)
5′-CUGCCGUAUAGGCAGC-3′
b. Csy4[H29A]
(SEQ ID No: 28)
MDHYLDIRLRPDPEFPPAQLMSVLFGKLAQALVAQGGDRIGVSFPDLDES
RSRLGERLRIHASADDLRALLARPWLEGLRDHLQFGEPAVVPHPTPYRQV
SRVQAKSNPERLRRRLMRRHDLSEEEARKRIPDTVARALDLPFVTLRSQS
TGQHFRLFIRHGPLQVTAEEGGFTCYGLSKGGFVPWF

In each of the aforementioned sequences, one may, for example, use the identical sequence or sequences that have one or more insertions, deletions or substitutions in one or both sequences of a binding pair. By way of a non-limiting example, for either or both members of a binding pair one may use a sequence that is at least 80%, at least 85%, at least 90%, at least 95%, or at least 98% the same as an aforementioned sequence.

Additional Chemistries

In some embodiments, the base-editing complexes of the present invention are combined with additional chemistry technologies. For example, in some embodiments, a base editing complex further comprises a cysteine/selenocysteine tag. In some embodiments, the base editing complex comprises or is associated with elements for cycloaddition via click chemistry.

Methods For Base-Editing

In another embodiment, the present invention provides methods for base editing. In these methods, one exposes a base editing complex of the present invention to double-stranded DNA or to a solution that contains dsDNA or to a cell that contains dsDNA or to a subject. The method may occur in vitro or be conducted in vivo or ex vivo and may comprise delivering the base editing complex to a subject as part of a medicament for treatment.

These methods may, for example, be used to modify an immune cell selected from a T cell (including a primary T cell), Natural Killer (NK cell), B cell, or CD34+ hematopoietic stem progenitor cell (HSPC). The immune cell may be an engineered immune cell, such as T-cell comprising a CAR or TCR. The methods herein may thus be applied to engineer further a cell that has already been modified to include a CAR and/or TCR that is useful in therapy. By way of further example, primary immune cells, either naturally occurring within a host animal or patient, or derived from a stem cell or an induced pluripotent stem cell (iPSC) may be genetically modified using the methods and complexes provided herein. Suitable stem cells include, but are not limited to, mammalian stem cells such as human stem cells, including, but not limited to, hematopoietic, neural, embryonic, induced pluripotent stem cells (iPSC), mesenchymal, mesodermal, liver, pancreatic, muscle, and retinal stem cells. Other stems cells include, but are not limited to, mammalian stem cells such as mouse stem cells, e.g., mouse embryonic stem cells.

Provided herein are also methods for genome engineering (e.g., altering or manipulating the expression of one or more genes or one or more gene products) in prokaryotic or eukaryotic cells, in vitro, in vivo, or ex vivo. In particular, the methods provided herein may be useful for targeted base editing disruption in mammalian cells including primary human T cells, natural killer (NK) cells, CD34+ hematopoietic stem and progenitor cells (HSPCs), such as HSPCs isolated from umbilical cord blood or bone marrow and cells differentiated from them, as well as HSPCs isolated from mobilized peripheral blood.

Also provided herein are genetically engineered cells arising from haematopoietic stem cells, such as T cells, that have been modified according to the methods described herein.

In some cases, the methods are configured to produce genetically engineered T cells arising from HSCs or iPSCs, that are suitable as “universally acceptable” cells for therapeutic application. Haemopoietic stem cells (HSCs) arise from hemangioblasts, which can give rise to HSCs, vascular smooth muscle cells and angioblasts, which differentiate into vascular endothelial cells. HSCs can give rise to common myeloid and common lymphoid progenitors from which arise T cells, Natural Killer (NK) cells, B cells, myeloblasts, erythroblasts and other cells involved in the production of cells of blood, bone marrow, spleen, lymph nodes, and thymus. Such methods can also be applied to natural killer (NK) cells, CD34+ hematopoietic stem and progenitor cells (HSPCs), such as HSPCs isolated from umbilical cord blood or bone marrow and cells differentiated from them, as well as HSPCs isolated from mobilized peripheral blood.

In another aspect, provided herein are methods for targeting diseases for base editing correction. In some of the methods, the base editing complexes are delivered to a subject for treatment. The target sequence can be any disease-associated polynucleotide or gene. Examples of useful applications of mutation or correction of an endogenous gene sequence according to the present invention include but are not limited to: alterations of disease-associated gene mutations, alterations in sequences encoding splice sites, alterations in regulatory sequences, alterations in sequences to cause a gain-of-function mutation, and/or alterations in sequences to cause a loss-of-function mutation, and targeted alterations of sequences encoding structural characteristics of a protein.

Delivery of Components Into Cells

The base editing complexes or their components may be delivered to target cells and organisms via various methods and various formats (DNA, RNA or protein) or combination of these different formats. The base editing components may be delivered as: (a) DNA polynucleotides that encode the relevant sequence for the protein effectors or the guide RNAs; (b) synthetic RNA encoding the sequence for the protein effectors (messenger RNA) or the guide RNAs; (c) purified protein for the effector. When delivering as protein format, the Type V Cas protein can be assembled with the guide RNAs to form a ribonucleoprotein complex (RNP) for delivery into target cells and organisms.

For example, the components or complexes as assembled may be delivered together or separately by electroporation, by nucleofection, by transfection, via nanoparticles, via viral mediated RNA delivery, via non-viral mediated delivery, via extracellular vesicles (for example, exosome and microvesicles), via eukaryotic cell transfer (for example, by recombinant yeast) and other methods that can package molecules such that they can be delivered to a target viable cell without changes to the genomic landscape.

Other methods include, but are not limited to, non-integrative transient transfer of DNA. polynucleotides that include the relevant sequence for the protein recruitment so that the molecule can be transcribed into the desired RNA molecule and for amino acid containing components translated into a protein or protein fragment. This includes, without limitation, DNA-only vehicles (for example, plasmids, MiniCircles, MiniVectors, MiniStrings, Protelomerase generated DNA molecules (for example Doggybones), artificial chromosome (for example HAC), and cosmids), via DNA vehicles by nanoparticies, extracellular vesicles (for example, exosome and microvesicles), via eukaryotic cell transfer (for example, by recombinant yeast), transient viral transfer by AAV, non-integrating viral particles (for example, lentivirus and retrovirus based systems), cell penetrating peptides and other technology that can mediate the introduction of DNA into a cell without direct integration into the genomic landscape. Another method for the introduction of the RNA components include the use of integrative gene transfer technology for stable introduction of the machinery for RNA transcription into the genome of the target cells, this can be controls via constitutive or promoter inducible systems to attenuate the RNA expression and this can also be designed so that the system can be removed after the utility has been met for example, introducing a Cre-Lox recombination system), such technology for stable gene transfer includes, but is not limited to, integrating viral particles (for example, lentivirus, adenovirus and retrovirus based systems), transposase mediate transfer (for example Sleeping Beauty and Piggybac), exploitation of the non-homologous repair pathways introduced by DNA breaks (for example, utilizing CRISPR and TALEN) technology and a surrogate DNA molecule, and other technology that encourages integration of the target DNA into a cell of interest.

The various components of the complexes of the present invention, if not synthesized enzymatically within a cell or solution, may be created chemically or, if naturally occurring, isolated and purified from naturally occurring sources. Methods for chemically and enzymatically synthesizing the various embodiments of the present invention are well known to persons of ordinary skill in the art. Similarly, methods for ligating or introducing covalent bonds between components of the present invention are also well known to persons of ordinary skill in the art.

Applications

By way of a non-limiting example, the complexes of the present invention may be used to recruit transcriptional activators such as p65 and V64, as well as moieties that introduce epigenetic modifications or affect HDR. The complexes of the present invention can also be used for the following applications; base editing, genome editing, genome screening, generation of therapeutic cells, genome tagging, epigenome editing, karyotype engineering, chromatin imaging, transcriptome and metabolic pathway engineering, genetic circuits engineering, cell signaling sensing, cellular events recording, lineage information reconstruction, gene drive, DNA genotyping, miRNA quantification, in vivo cloning, site-directed mutagenesis, genomic diversification, and proteomic analysis in situ. In some embodiments, a cell or a population of cells are exposed to a base editing complex of the present invention and the cell or cells are introduced to a subject by infusion.

Applications also include research of human diseases such as cancer immunotherapy, antiviral therapy, bacteriophage therapy, cancer diagnosis, pathogen screening, microbiota remodeling, stem-cell reprogramming, immunogenomic engineering, vaccine development, and antibody production.

### EXAMPLES

### Example 1: Transfection of Plasmid Components For dCasPhi Base Editing

Vector Construction:

The coding sequence for a deactivated version of CasPhi and 2xUGI fusion (dCasPhi-2xUGI) was obtained and cloned into an expression vector under the control of the mouse CMV promoter in a T2A polycistronic cassette with a red fluorescent protein-puromycin fusion. The coding sequence for MS2 coat protein lizard Anolis Apobec fusion (MCP-lizAnoA1) (“AnoA1”) is:

(SEQ ID NO: 139)
ATGGCCCCCAAGAAGAAGCGGAAAGTGATGGAGCCGGAGGCTTTTCAGCG
CAACTTTGACCCTCGGGAATTTGCCGCCTGTACACTCCTCTTGTATGAGA
TCCACTGGGACAATAACACATCTAGAAATTGGTGTACGAATAAGCCTGGG
CTCCACGCTGAGGAGAATTTCTTGCAGATATTTAATGAGAAAATTGACAT
TAAACAGGATACGCCGTGCTCTATAACATGGTTCCTTTCTTGGAGCCCCT
GTTACCCTTGTAGCCAAGCAATAATAAAATTCTTGGAGGCACACCCGAAT
GTCAGTCTGGAGATTAAGGCTGCGCGGCTGTATATGCATCAAATAGACTG
TAACAAGGAGGGACTCAGAAATCTGGGCCGGAATCGAGTGTCAATAATGA
ACCTGCCTGATTATAGGCATTGCTGGACTACGTTTGTTGTGCCAAGGGGA
GCAAACGAAGATTACTGGCCACAAGACTTTCTGCCTGCGATCACAAATTA
CTCCCGAGAACTCGACTCCATACTGCAGGATGAGCTGAAGACACCCCTGG
GCGACACCACACACACCTCTCCACCTTGCCCAGCACCAGAGCTGCTGGGA
GGCCCTATGGCCAGCAACTTCACACAGTTTGTGCTGGTGGATAATGGAGG
AACCGGCGACGTGACAGTGGCACCATCTAACTTTGCCAATGGCATCGCCG
AGTGGATCAGCTCCAACTCTCGGAGCCAGGCCTATAAGGTGACCTGTAGC
GTGCGGCAGTCTAGCGCCCAGAATAGAAAGTATACAATCAAGGTGGAGGT
GCCTAAGGGCGCCTGGAGATCCTACCTGAACATGGAGCTGACCATCCCAA
TCTTTGCCACAAATTCTGATTGCGAGCTGATCGTGAAGGCCATGCAGGGC
CTGCTGAAGGACGGCAACCCTATCCCAAGCGCCATCGCCGCCAATAGCGG
AATCTACACGCGTAAAATCAGCCTCGACTGTGCCTTCTAG,

which was obtained and cloned into an expression vector under control of the mouse CMV promoter. The sequence for crRNA containing the MS2 ligand binding moiety and unique spacer regions were cloned into an expression vector under control of the hU6 promoter.

HEK 293T cells (ATCC, #CRL-11268) were seeded at 20,000 cells per well in a 96-well plate one day prior to transfection. Cells were co-transfected using DharmaFECT Duo Transfection Reagent (Horizon Discovery, #T-2010) and 200 ng dCasPhi-2xUGI plasmid, 100 ng AnoA1 plasmid, and 100 ng crRNA plasmid. The plasmid crRNA consisted of a direct repeat length of e.g., 35 nucleotides and different spacer sequences of 18 or 20 nucleotides targeting transcripts within Site2 or B2M gene targets. Additionally, they have the MS2 ligand binding moiety at the 5′ terminus, the 3′ terminus, internally not at either the 5′ or 3′ terminus, or combinations therein. See SEQ ID NO: 35 to 58 in Table 3 below.

Cell Processing

Cells were lysed in 100 μL of a buffer containing proteinase K (Thermo Scientific, #FEREO0492), RNase A (Thermo Scientific, #FEREN0531), and Phusion HF buffer (Thermo Scientific, #F-518L) for 30 min at 56° C., followed by a 5 min heat inactivation at 95° C. This cell lysate was used to generate PCR amplicons spanning the region containing the base editing site(s). PCR amplicons between 400-1000 bp in length were sequenced by Sanger sequencing.

Editing Analysis

Base editing efficiencies were calculated using the Chimera analysis tool, an adaptation of the open source tool BEAT. Chimera determines editing efficiency by first subtracting the background noise to define the expected variability in a sample. This allows the estimation of editing efficiency without the need to normalize to control samples. Following this, Chimera filters out any outliers from the noise using the Median Absolute Deviation (MAD) method and then assesses the editing efficiency of the base editor over the span of the 18-20 bp input guide sequence.

Table 3 provides a list of plasmid guide sequences. Spacer region sequences are in bold. Direct repeat sequences are underlined. The ligand binding moiety sequence is italicized.

TABLE 3

| Seq. | | ligand | |
|---|---|---|---|
| ID | | binding | crRNA |
| NO | Full Sequence | moiety | sequence |
| 35 | CTTTCAAGACTAATAGATTGCTCCTTACGAGGA | MS2-less | Site2 |
| | GACAGGCTGGCCCGCCCCGCA | | gRNA4 |
| 36 | CTTTCAAGACTAATAGATTGCTCCTTACGAGGA | MS2-less | Site2 |
| | GACGTGTTCCAGTTTCCTTTA | | gRNA5 |
| 37 | CGCACATGAGGATCACCCATGTGCCTTTCAAGAC | 5′MS2 | Site2 |
| | TAATAGATTGCTCCTTACGAGGAGACAGGCTG | pre-cr | gRNA4 |
| | GCCCGCCCCGCA | | |
| 38 | CGCACATGAGGATCACCCATGTGCCTTTCAAGAC | 5′MS2 | Site2 |
| | TAATAGATTGCTCCTTACGAGGAGACGTGTTCC | pre-cr | gRNA5 |
| | AGTTTCCTTTA | | |
| 39 | CTTTCAAGACTGCGCACATGAGGATCACCCATGT | Embedded | Site2 |
| | GCAATAGATTGCTCCTTACGAGGAGACAGGCT | 5′MS2 | gRNA4 |
| | GGCCCGCCCCGCA | pre-cr | |
| 40 | CTTTCAAGACTGCGCACATGAGGATCACCCATGT | Embedded | Site2 |
| | GCAATAGATTGCTCCTTACGAGGAGACGTGTTC | 5′MS2 | gRNA5 |
| | CAGTTTCCTTTA | pre-cr | |
| 41 | CTTTCAAGACTAATAGATTGCTCCTTACAACATG | Loop MS2 | Site2 |
| | AGGATCACCCATGTTGCGAGGAGACAGGCTGGC | | gRNA4 |
| | CCGCCCCGCA | | |
| 42 | CTTTCAAGACTAATAGATTGCTCCTTACAACATG | Loop MS2 | Site2 |
| | AGGATCACCCATGTTGCGAGGAGACGTGTTCCA | | gRNA5 |
| | GTTTCCTTTA | | |
| 43 | CTTTCAAGACTAATAGATTGCTCCTTAACAACAT | Loop MS2 | Site2 |
| | GAGGATCACCCATGTTGCCGAGGAGACAGGCTG | with | gRNA4 |
| | GCCCGCCCCGCA | extension | |
| 44 | CTTTCAAGACTAATAGATTGCTCCTTAACAACAT | Loop MS2 | Site2 |
| | GAGGATCACCCATGTTGCCGAGGAGACGTGTTC | with | gRNA5 |
| | CAGTTTCCTTTA | extension | |
| 45 | CTTTCAAGACTAATAGATTGCTCCTTACGAGGA | 3′MS2 | Site2 |
| | GACAGGCTGGCCCGCCCCGCAGCGCACATGAG | | gRNA4 |
| | GATCACCCATGTGC | | |
| 46 | CTTTCAAGACTAATAGATTGCTCCTTACGAGGA | 3′MS2 | Site2 |
| | GACGTGTTCCAGTTTCCTTTAGCGCACATGAGG | | gRNA5 |
| | ATCACCCATGTGC | | |
| 47 | AATAGATTGCTCCTTACGAGGAGACAGGCTGG | MS2-less | Site2 |
| | CCCGCCCCGCA | no pre- | gRNA4 |
| | | crRNA | |
| 48 | AATAGATTGCTCCTTACGAGGAGACGTGTTCCA | MS2-less | Site2 |
| | GTTTCCTTTA | no pre- | gRNA5 |
| | | crRNA | |
| 49 | GCACATGAGGATCACCCATGTGCAATAGATTGCT | 5′ MS2, | Site2 |
| | CCTTACGAGGAGACAGGCTGGCCCGCCCCGC | no pre- | gRNA4 |
| | A | crRNA | |
| 50 | GCACATGAGGATCACCCATGTGCAATAGATTGCT | 5′ MS2, | Site2 |
| | CCTTACGAGGAGACGTGTTCCAGTTTCCTTTA | no pre- | |
| | | crRNA | gRNA5 |
| 51 | CTTTCAAGACTAATAGATTGCTCCTTACGAGGA | MS2-less | B2M |
| | GACAGGAATGCCCGCCAGCGC | | gRNA6 |
| 52 | CGCACATGAGGATCACCCATGTGCCTTTCAAGAC | 5′MS2 | B2M |
| | TAATAGATTGCTCCTTACGAGGAGACAGGAAT | pre-cr | gRNA6 |
| | GCCCGCCAGCGC | | |
| 53 | CTTTCAAGACTAATAGATTGCTCCTTACGAGGA | MS2-less | Site2 |
| | GACAGGCTGGCCCGCCCCGCAGT | | gRNA4_2 |
| | | | 0nt spacer |
| 54 | CTTTCAAGACTAATAGATTGCTCCTTACGAGGA | MS2-less | Site2 |
| | GACGTGTTCCAGTTTCCTTTACA | | gRNA5_2 |
| | | | 0nt spacer |
| 55 | CTTTCAAGACTAATAGATTGCTCCTTACGAGGA | MS2-less | B2M |
| | GACAGGAATGCCCGCCAGCGCGA | | gRNA6_2 |
| | | | 0nt spacer |
| 56 | CGCACATGAGGATCACCCATGTGCCTTTCAAGAC | 5′MS2 | Site2 |
| | TAATAGATTGCTCCTTACGAGGAGACAGGCTG | pre-cr | gRNA4_2 |
| | GCCCGCCCCGCAGT | | 0nt spacer |
| 57 | CGCACATGAGGATCACCCATGTGCCTTTCAAGAC | 5′MS2 | Site2 |
| | TAATAGATTGCTCCTTACGAGGAGACGTGTTCC | pre-cr | gRNA5_2 |
| | AGTTTCCTTTACA | | 0nt spacer |
| 58 | CGCACATGAGGATCACCCATGTGCCTTTCAAGAC | 5′MS2 | B2M |
| | TAATAGATTGCTCCTTACGAGGAGACAGGAAT | pre-cr | gRNA6_2 |
| | GCCCGCCAGCGCGA | | 0nt spacer |

FIGS. 1A to 1G provide guide RNA folds predictions using Geneious software of a CasPhi guide RNA, with the following parameters:
without MS2 aptamer, SEQ ID NO: 59, FIG. 1A;
with MS2 aptamer 5′ of the pre-cr, SEQ ID NO: 60, FIG. 1B;
with MS2 aptamer embedded in the pre-cr, SEQ ID NO: 61, FIG. 1C;
with MS2 aptamer in the loop, SEQ ID NO: 62, FIG. 1D;
with MS2 aptamer at the 3′ end of the guide, SEQ ID NO: 63, FIG. 1E;
without MS2 sequence and without pre-cr, SEQ ID NO: 64, FIG. 1F; and
with MS2 sequence 5′ of the crRNA, SEQ ID NO: 65, FIG. 1G.
   In these figures, spacer sequences are shown bound to an oligonucleotide that does not make up the gRNA: TAAAGGAAACTGGAACAC (SEQ ID NO: 66).

The inventors used sequences from Table 3 to evaluate the effects of MS2 placement on base editing levels at two genomic target sites shown. Editing results are shown for eleven target C residues, which are identified in FIG. 2A and FIG. 2B by the location of the C residue.

For these experiments, guide RNAs targeting: (A) Site2 gRNA4, SEQ ID NO:s 35, 37, 39, 45, 47, and 49 (see FIG. 2A); and (B) Site2 gRNA5, SEQ ID NO:s 36, 38, 40, 46, 48, and 50 (see FIG. 2B) with different placement of MS2 aptamers were compared for C>T base editing efficiency. The aforementioned guide RNA plasmids, dCasPhi plasmid and deaminase plasmid (liz AnoA1) were co-transfected in HEK293T cells. Cells were analyzed for base editing using Chimera software. The data show % C>T conversion at the indicated cytosines along the spacer sequence.

Based on the data, 5′MS2 pre-cr configuration results in higher levels of base editing at both sites, compared to other MS2 placements. Thus, for a CasPhi system a gRNA may encode SEQ ID NO 137 and/or SEQ ID NO 138 (see FIG. 4E and FIG. 4F).

### Example 2: Transfection of Plasmid Components For CasPhi (Cas12j) DNA Cutting

Vector Construction

The coding sequence for CasPhi was obtained and cloned into an expression vector under the control of the mouse CMV promoter in a T2A polycistronic cassette with a red fluorescent protein-puromycin fusion. The sequence for crRNA and unique spacer regions were cloned into an expression vector under control of the hU6 promoter.

HEK 293T cells (ATCC, #CRL-11268) were seeded at 20,000 cells per well in a 96-well plate one day prior to transfection. Cells were co-transfected using DharmaFECT Duo Transfection Reagent (Horizon Discovery, #T-2010) and 200 ng CasPhi plasmid, and 100 ng crRNA plasmid. The plasmid crRNA consisted of a direct repeat length of 35 nucleotides.

Cell Processing:

Cells were lysed in 100 μL of a buffer containing proteinase K (Thermo Scientific, #FEREO0492), RNase A (Thermo Scientific, #FEREN0531), and Phusion HF buffer (Thermo Scientific, #F-518L) for 30 min at 56° C., followed by a 5 min heat inactivation at 95° C. This cell lysate may be used to generate PCR amplicons spanning the region containing the base editing site(s). PCR amplicons between 400-1000 bp in length may be digested with T7 endonuclease I (T7EI) enzyme (NEB, M0302S) in the presence of NEB buffer 2 (NEB, B7002S) for 25 minutes. The digested PCR product may be run on 2% agarose gel for 90 minutes at 80 volts, imaged, and analyzed using Horizon Discovery's online T7EI calculator

(https://horizondiscovery.com/en/ordering-and-calculation-tools/t7ei-calculator).

SEQ ID NO: 36, 38, 40, 42, 44, and 46 from table 3 were used in this example.

The percentages of editing from C to T are shown in FIG. 2C and FIG. 2D. These figures demonstrate that inclusion of MS2 at the 5′ pre-cr, embedded 5′ pre-cr and 3′ positions of Site2 gRNA5 does not affect the ability of the CasPhi to cause indel formation.

### Example 3: Base Editing With Guides For Multiple Sets of Deactivated CasPhi Mutants

Different deactivating mutations of CasPhi were compared for base editing efficiency—D369A, E566A, and D369A/E566A/D658A. HEK293T cells were transfected with dCasPhi-2xUGI plasmid+AnoA1 plasmid+the indicated plasmid gRNAs, 35, 37, 39, and 45 for Site2 gRNA4 (FIG. 3A), and 36, 38, 40, and 46 for Site2 gRNA5 (FIG. 3B) The cells were harvested, and base editing levels were analyzed using Chimera software. The data, which are summarized in FIG. 3A and FIG. 3B, show % C>T conversion at the indicated cytosine positions along the spacers.

These data demonstrate the capability of using dCasPhi 5′ MS2 pre-cr guides with different deactivating mutations to perform base editing at several different target C residues at HEK Site2. Table 4 provides deactivated CasPhi sequences.

TABLE 4

| SEQ ID NO | Mutation | Sequence |
|---|---|---|
| 123 | D369A | ATGGTCGACGGGAGCGGGCCGGCAGCTAAACGGGTGAAGTT |
| | | GGACAGTGGTGGAATTAAACCTACAGTTTCTCAGTTTCTTACC |
| | | CCTGGTTTTAAGCTGATAAGAAACCATAGTCGGACGGCTGGA |
| | | CTTAAGCTGAAGAATGAGGGCGAAGAGGCATGCAAGAAGTT |
| | | CGTACGGGAGAACGAAATTCCCAAAGATGAATGTCCAAACTT |
| | | TCAAGGTGGACCCGCAATCGCGAACATTATAGCCAAGAGTCG |
| | | CGAATTTACCGAGTGGGAAATATATCAAAGTTCACTGGCGAT |
| | | CCAAGAGGTGATTTTCACCTTGCCGAAGGATAAGCTGCCCGA |
| | | GCCTATACTCAAGGAAGAATGGCGCGCCCAATGGTTGAGCGA |
| | | ACACGGCCTCGATACGGTGCCTTACAAGGAAGCTGCCGGACT |
| | | TAATTTGATAATTAAGAACGCGGTCAACACTTACAAAGGGGT |
| | | CCAGGTGAAAGTCGATAATAAGAATAAGAACAACCTGGCCA |
| | | AAATCAACCGCAAGAACGAAATCGCGAAATTGAACGGCGAA |
| | | CAAGAAATCAGCTTCGAAGAGATCAAAGCCTTCGATGATAAA |
| | | GGATATCTCCTGCAAAAGCCAAGTCCGAATAAGAGCATATAT |
| | | TGCTACCAAAGCGTGTCTCCAAAGCCATTCATAACCTCTAAA |
| | | TACCATAACGTGAATCTGCCCGAAGAATATATCGGCTACTAC |
| | | CGCAAGTCAAACGAGCCCATCGTTAGTCCCTATCAATTCGAT |
| | | AGATTGCGAATCCCAATTGGCGAACCCGGATATGTACCAAAA |
| | | TGGCAGTATACCTTTCTGTCTAAGAAAGAGAATAAGCGGAGA |
| | | AAGCTCTCCAAGCGGATTAAGAATGTTAGTCCTATTCTTGGG |
| | | ATAATATGCATTAAGAAAGACTGGTGCGTATTCGATATGAGG |
| | | GGCCTGCTCAGAACGAACCACTGGAAGAAATACCATAAACC |
| | | GACAGATTCTATCAATGACCTCTTCGATTATTTCACTGGAGAC |
| | | CCTGTAATCGACACGAAAGCGAACGTCGTCCGATTCAGATAT |
| | | AAAATGGAAAATGGCATTGTTAATTACAAGCCGGTGCGCGAA |
| | | AAGAAAGGCAAGGAACTTTTGGAAAACATATGTGATCAAAA |
| | | TGGGAGCTGTAAGTTGGCCACTGTGGCCGTTGGTCAAAACAA |
| | | CCCAGTGGCAATTGGACTGTTTGAACTTAAGAAAGTAAATGG |
| | | TGAACTTACCAAAACCTTGATTTCACGGCATCCTACTCCGATC |
| | | GACTTTTGTAATAAAATTACGGCTTACAGGGAGCGGTATGAT |
| | | AAGCTCGAATCCAGCATCAAGTTGGATGCCATAAAGCAATTG |
| | | ACATCTGAGCAAAAGATCGAAGTTGATAACTATAACAATAAT |
| | | TTTACCCCTCAAAACACTAAGCAGATAGTGTGCAGCAAGCTC |
| | | AATATCAATCCAAACGACCTTCCTTGGGATAAAATGATTTCT |
| | | GGGACTCATTTCATTAGCGAGAAAGCCCAAGTCAGTAATAAA |
| | | TCAGAAATATACTTCACATCTACCGATAAGGGGAAAACTAAG |
| | | GACGTAATGAAGAGCGACTACAAGTGGTTTCAAGACTATAAA |
| | | CCAAAACTGTCAAAGGAAGTAAGGGACGCACTCAGCGATATT |
| | | GAATGGCGGCTTAGGAGAGAAAGTCTTGAATTTAACAAATTG |
| | | AGTAAATCACGGGAACAAGATGCACGGCAACTGGCCAATTG |
| | | GATCTCTTCCATGTGTGATGTTATCGGAATAGAGAACCTGGT |
| | | GAAGAAGAACAATTTCTTTGGTGGAAGCGGCAAGAGGGAAC |
| | | CGGGGTGGGACAACTTCTATAAACCGAAGAAGGAGAATCGA |
| | | TGGTGGATCAACGCAATTCATAAAGCTCTCACAGAACTCTCT |
| | | CAAAACAAAGGGAAAAGAGTGATTCTCTTGCCAGCAATGAG |
| | | AACATCTATCACATGCCCTAAATGTAAGTACTGTGACAGCAA |
| | | GAACCGGAACGGCGAGAAGTTCAATTGTCTGAAGTGTGGCAT |
| | | AGAACTCAACGCAGACATTGATGTTGCTACCGAAAATCTCGC |
| | | GACCGTTGCTATTACCGCGCAAAGTATGCCTAAACCCACCTG |
| | | TGAGAGGAGTGGTGATGCCAAGAAGCCCGTACGTGCACGAA |
| | | AGGCAAAGGCGCCAGAATTTCATGACAAACTCGCGCCCTCAT |
| | | ACACAGTTGTCTTGCGCGAAGCTGTTTAATGA |
| 124 | E566A | ATGGTCGACGGGAGCGGGCCGGCAGCTAAACGGGTGAAGTT |
| | | GGACAGTGGTGGAATTAAACCTACAGTTTCTCAGTTTCTTACC |
| | | CCTGGTTTTAAGCTGATAAGAAACCATAGTCGGACGGCTGGA |
| | | CTTAAGCTGAAGAATGAGGGCGAAGAGGCATGCAAGAAGTT |
| | | CGTACGGGAGAACGAAATTCCCAAAGATGAATGTCCAAACTT |
| | | TCAAGGTGGACCCGCAATCGCGAACATTATAGCCAAGAGTCG |
| | | CGAATTTACCGAGTGGGAAATATATCAAAGTTCACTGGCGAT |
| | | CCAAGAGGTGATTTTCACCTTGCCGAAGGATAAGCTGCCCGA |
| | | GCCTATACTCAAGGAAGAATGGCGCGCCCAATGGTTGAGCGA |
| | | ACACGGCCTCGATACGGTGCCTTACAAGGAAGCTGCCGGACT |
| | | TAATTTGATAATTAAGAACGCGGTCAACACTTACAAAGGGGT |
| | | CCAGGTGAAAGTCGATAATAAGAATAAGAACAACCTGGCCA |
| | | AAATCAACCGCAAGAACGAAATCGCGAAATTGAACGGCGAA |
| | | CAAGAAATCAGCTTCGAAGAGATCAAAGCCTTCGATGATAAA |
| | | GGATATCTCCTGCAAAAGCCAAGTCCGAATAAGAGCATATAT |
| | | TGCTACCAAAGCGTGTCTCCAAAGCCATTCATAACCTCTAAA |
| | | TACCATAACGTGAATCTGCCCGAAGAATATATCGGCTACTAC |
| | | CGCAAGTCAAACGAGCCCATCGTTAGTCCCTATCAATTCGAT |
| | | AGATTGCGAATCCCAATTGGCGAACCCGGATATGTACCAAAA |
| | | TGGCAGTATACCTTTCTGTCTAAGAAAGAGAATAAGCGGAGA |
| | | AAGCTCTCCAAGCGGATTAAGAATGTTAGTCCTATTCTTGGG |
| | | ATAATATGCATTAAGAAAGACTGGTGCGTATTCGATATGAGG |
| | | GGCCTGCTCAGAACGAACCACTGGAAGAAATACCATAAACC |
| | | GACAGATTCTATCAATGACCTCTTCGATTATTTCACTGGAGAC |
| | | CCTGTAATCGACACGAAAGCGAACGTCGTCCGATTCAGATAT |
| | | AAAATGGAAAATGGCATTGTTAATTACAAGCCGGTGCGCGAA |
| | | AAGAAAGGCAAGGAACTTTTGGAAAACATATGTGATCAAAA |
| | | TGGGAGCTGTAAGTTGGCCACTGTGGATGTTGGTCAAAACAA |
| | | CCCAGTGGCAATTGGACTGTTTGAACTTAAGAAAGTAAATGG |
| | | TGAACTTACCAAAACCTTGATTTCACGGCATCCTACTCCGATC |
| | | GACTTTTGTAATAAAATTACGGCTTACAGGGAGCGGTATGAT |
| | | AAGCTCGAATCCAGCATCAAGTTGGATGCCATAAAGCAATTG |
| | | ACATCTGAGCAAAAGATCGAAGTTGATAACTATAACAATAAT |
| | | TTTACCCCTCAAAACACTAAGCAGATAGTGTGCAGCAAGCTC |
| | | AATATCAATCCAAACGACCTTCCTTGGGATAAAATGATTTCT |
| | | GGGACTCATTTCATTAGCGAGAAAGCCCAAGTCAGTAATAAA |
| | | TCAGAAATATACTTCACATCTACCGATAAGGGGAAAACTAAG |
| | | GACGTAATGAAGAGCGACTACAAGTGGTTTCAAGACTATAAA |
| | | CCAAAACTGTCAAAGGAAGTAAGGGACGCACTCAGCGATATT |
| | | GAATGGCGGCTTAGGAGAGAAAGTCTTGAATTTAACAAATTG |
| | | AGTAAATCACGGGAACAAGATGCACGGCAACTGGCCAATTG |
| | | GATCTCTTCCATGTGTGATGTTATCGGAATAGCCAACCTGGTG |
| | | AAGAAGAACAATTTCTTTGGTGGAAGCGGCAAGAGGGAACC |
| | | GGGGTGGGACAACTTCTATAAACCGAAGAAGGAGAATCGAT |
| | | GGTGGATCAACGCAATTCATAAAGCTCTCACAGAACTCTCTC |
| | | AAAACAAAGGGAAAAGAGTGATTCTCTTGCCAGCAATGAGA |
| | | ACATCTATCACATGCCCTAAATGTAAGTACTGTGACAGCAAG |
| | | AACCGGAACGGCGAGAAGTTCAATTGTCTGAAGTGTGGCATA |
| | | GAACTCAACGCAGACATTGATGTTGCTACCGAAAATCTCGCG |
| | | ACCGTTGCTATTACCGCGCAAAGTATGCCTAAACCCACCTGT |
| | | GAGAGGAGTGGTGATGCCAAGAAGCCCGTACGTGCACGAAA |
| | | GGCAAAGGCGCCAGAATTTCATGACAAACTCGCGCCCTCATA |
| | | CACAGTTGTCTTGCGCGAAGCTGTTTAATGA |
| 134 | D369A, | ATGGTCGACGGGAGCGGGCCGGCAGCTAAACGGGTGAAGTT |
| | E566A & | GGACAGTGGTGGAATTAAACCTACAGTTTCTCAGTTTCTTACC |
| | D658A | CCTGGTTTTAAGCTGATAAGAAACCATAGTCGGACGGCTGGA |
| | | CTTAAGCTGAAGAATGAGGGCGAAGAGGCATGCAAGAAGTT |
| | | CGTACGGGAGAACGAAATTCCCAAAGATGAATGTCCAAACTT |
| | | TCAAGGTGGACCCGCAATCGCGAACATTATAGCCAAGAGTCG |
| | | CGAATTTACCGAGTGGGAAATATATCAAAGTTCACTGGCGAT |
| | | CCAAGAGGTGATTTTCACCTTGCCGAAGGATAAGCTGCCCGA |
| | | GCCTATACTCAAGGAAGAATGGCGCGCCCAATGGTTGAGCGA |
| | | ACACGGCCTCGATACGGTGCCTTACAAGGAAGCTGCCGGACT |
| | | TAATTTGATAATTAAGAACGCGGTCAACACTTACAAAGGGGT |
| | | CCAGGTGAAAGTCGATAATAAGAATAAGAACAACCTGGCCA |
| | | AAATCAACCGCAAGAACGAAATCGCGAAATTGAACGGCGAA |
| | | CAAGAAATCAGCTTCGAAGAGATCAAAGCCTTCGATGATAAA |
| | | GGATATCTCCTGCAAAAGCCAAGTCCGAATAAGAGCATATAT |
| | | TGCTACCAAAGCGTGTCTCCAAAGCCATTCATAACCTCTAAA |
| | | TACCATAACGTGAATCTGCCCGAAGAATATATCGGCTACTAC |
| | | CGCAAGTCAAACGAGCCCATCGTTAGTCCCTATCAATTCGAT |
| | | AGATTGCGAATCCCAATTGGCGAACCCGGATATGTACCAAAA |
| | | TGGCAGTATACCTTTCTGTCTAAGAAAGAGAATAAGCGGAGA |
| | | AAGCTCTCCAAGCGGATTAAGAATGTTAGTCCTATTCTTGGG |
| | | ATAATATGCATTAAGAAAGACTGGTGCGTATTCGATATGAGG |
| | | GGCCTGCTCAGAACGAACCACTGGAAGAAATACCATAAACC |
| | | GACAGATTCTATCAATGACCTCTTCGATTATTTCACTGGAGAC |
| | | CCTGTAATCGACACGAAAGCGAACGTCGTCCGATTCAGATAT |
| | | AAAATGGAAAATGGCATTGTTAATTACAAGCCGGTGCGCGAA |
| | | AAGAAAGGCAAGGAACTTTTGGAAAACATATGTGATCAAAA |
| | | TGGGAGCTGTAAGTTGGCCACTGTGGCCGTTGGTCAAAACAA |
| | | CCCAGTGGCAATTGGACTGTTTGAACTTAAGAAAGTAAATGG |
| | | TGAACTTACCAAAACCTTGATTTCACGGCATCCTACTCCGATC |
| | | GACTTTTGTAATAAAATTACGGCTTACAGGGAGCGGTATGAT |
| | | AAGCTCGAATCCAGCATCAAGTTGGATGCCATAAAGCAATTG |
| | | ACATCTGAGCAAAAGATCGAAGTTGATAACTATAACAATAAT |
| | | TTTACCCCTCAAAACACTAAGCAGATAGTGTGCAGCAAGCTC |
| | | AATATCAATCCAAACGACCTTCCTTGGGATAAAATGATTTCT |
| | | GGGACTCATTTCATTAGCGAGAAAGCCCAAGTCAGTAATAAA |
| | | TCAGAAATATACTTCACATCTACCGATAAGGGGAAAACTAAG |
| | | GACGTAATGAAGAGCGACTACAAGTGGTTTCAAGACTATAAA |
| | | CCAAAACTGTCAAAGGAAGTAAGGGACGCACTCAGCGATATT |
| | | GAATGGCGGCTTAGGAGAGAAAGTCTTGAATTTAACAAATTG |
| | | AGTAAATCACGGGAACAAGATGCACGGCAACTGGCCAATTG |
| | | GATCTCTTCCATGTGTGATGTTATCGGAATAGCCAACCTGGTG |
| | | AAGAAGAACAATTTCTTTGGTGGAAGCGGCAAGAGGGAACC |
| | | GGGGTGGGACAACTTCTATAAACCGAAGAAGGAGAATCGAT |
| | | GGTGGATCAACGCAATTCATAAAGCTCTCACAGAACTCTCTC |
| | | AAAACAAAGGGAAAAGAGTGATTCTCTTGCCAGCAATGAGA |
| | | ACATCTATCACATGCCCTAAATGTAAGTACTGTGACAGCAAG |
| | | AACCGGAACGGCGAGAAGTTCAATTGTCTGAAGTGTGGCATA |
| | | GAACTCAACGCAGCCATTGATGTTGCTACCGAAAATCTCGCG |
| | | ACCGTTGCTATTACCGCGCAAAGTATGCCTAAACCCACCTGT |
| | | GAGAGGAGTGGTGATGCCAAGAAGCCCGTACGTGCACGAAA |
| | | GGCAAAGGCGCCAGAATTTCATGACAAACTCGCGCCCTCATA |
| | | CACAGTTGTCTTGCGCGAAGCTGTTAGCGGCGGGAGCGGCGG |
| | | GAGCGGGGGGAGCACTAATCTGAGCGACATCATTGAGAAGG |
| | | AGACTGGGAAACAGCTGGTCATTCAGGAGTCCATCCTGATGC |
| | | TGCCTGAGGAGGTGGAGGAAGTGATCGGCAACAAGCCAGAG |
| | | TCTGACATCCTGGTGCACACCGCCTACGACGAGTCCACAGAT |
| | | GAGAATGTGATGCTGCTGACCTCTGACGCCCCCGAGTATAAG |
| | | CCTTGGGCCCTGGTCATCCAGGATTCTAACGGCGAGAATAAG |
| | | ATCAAGATGCTGAGCGGAGGATCCGGAGGATCTGGAGGCAG |
| | | CACCAACCTGTCTGACATCATCGAGAAGGAGACAGGCAAGC |
| | | AGCTGGTCATCCAGGAGAGCATCCTGATGCTGCCCGAAGAAG |
| | | TCGAAGAAGTGATCGGAAACAAGCCTGAGAGCGATATCCTG |
| | | GTCCATACCGCCTACGACGAGAGTACCGACGAAAATGTGATG |
| | | CTGCTGACATCCGACGCCCCAGAGTATAAGCCCTGGGCTCTG |
| | | GTCATCCAGGATTCCAACGGAGAGAACAAAATCAAAATGCTG |
| | | TCTGGCGGCTCAAAAAGAACCGCCGACGGCAGCGAATTCGA |
| | | GCCCAAGAAGAAGAGGAAAGTCTAATGA |

### Example 4: Base Editing With Different Spacer Lengths

In one experiment, HEK293T cells were electroporated with dCasPhi-2xUGI mRNA+AnoA1 mRNA+the indicated extended length spacer synthetic gRNAs for Site2 gRNA5. SEQ ID NO: 101 and 102 (see Table 5). In a second experiment, HEK 293T cells were transfected with dCasPhi-2xUGI plasmid+AnoA1 plasmid+the indicated gRNA plasmids for Site2 gRNA5. SEQ ID NO: 36, 38, 54, and 57 (see Table 3). The cells were harvested, and base editing levels were analyzed using Chimera software. The data show % C>T conversion at the indicated cytosine positions along the spacers. The percentage of C to T editing for each experiment is shown in FIG. 4A and FIG. 4B, respectively.

gRNAs used in this example may more generally be represented by the schematics of FIGS. 4C to 4F:
FIG. 4C is a schematic of synthetic gRNA with 18 nt spacer with a spacer represented by Ns. (SEQ ID NO 135)
FIG. 4D is a schematic of synthetic gRNA with a 20 nt spacer represented by Ns. (SEQ ID NO 136)
FIG. 4E is a schematic of plasmid gRNA with 18 nt spacer. (SEQ ID NO 137)
FIG. 4F is a schematic of plasmid gRNA with 20 nt spacer. (SEQ ID NO 138)

These data show that base editing works when using different spacer lengths in a gRNA that contains a ligand binding moiety.

### Example 5: Electroporation of mRNA and Synthetic Fuides For dCasPhi (Cas12j) Base Editing

mRNA Preparation:

Messenger RNA were prepared from DNA vectors carrying the T7 promoter and the coding sequences for dCasPhi-2xUGI and AnoA1 following the standard protocols for mRNA in vitro transcription.

RNA Synthesis:

All crRNA were synthesized by Horizon Discovery using either 2′-acetoxy ethyl orthoester (2′-ACE) or 2′-tert-butyldimethylsilyl (2′-TBDMS) protection chemistries or by Agilent. Chemical modifications were included where noted. RNA oligos were 2′-depotected/desalted and purified by either high-performance liquid chromatography (HPLC) or polyacrylamide gel electrophoresis (PAGE). Oligos were resuspended in 10 mM Tris pH7.5 buffer prior to electroporation.

HEK 293T cells (ATCC, #CRL-11268) were electroporated using the Invitrogen™ Neon™ Transfection System, 10 μl Kit. A mixture of 50,000 cells, 650 ng of dCasPhi-2xUGI mRNA and 200 ng AnoA1 mRNA, and 6 μM of synthetic crRNA may be electroporated at 1150V for 20 ms and for 2 pulses. The chemically synthesized crRNA consisted of different direct repeat lengths of 21 or 35 nucleotides, different spacer sequences targeting transcripts within Site2 or B2M gene targets, and the MS2 ligand binding moiety at the 5′ terminus, the 3′ terminus, internally not at either the 5′ or 3′ terminus, or combinations therein. Each sequence optionally contained chemical modifications at one or more bases and within one or more linkages. Cells were plated in a 96-well plate with full serum media and harvested after 48-72 hours for further processing

Cell Processing

Cells were lysed in 100 μL of a buffer containing proteinase K (Thermo Scientific, #FEREO0492), RNase A (Thermo Scientific, #FEREN0531), and Phusion HF buffer (Thermo Scientific, #F-518L) for 30 min at 56° C., followed by a 5 min heat inactivation at 95° C. This cell lysate was used to generate PCR amplicons spanning the region containing the base editing site(s). PCR amplicons between 400-1000 bp in length may be sequenced by Sanger sequencing. PCR amplicons may be purified (Qiagen, #28181) and submitted for NGS sequencing.

Editing Analysis

Base editing efficiencies were calculated using the Chimera analysis tool, an adaptation of the open source tool BEAT. Chimera determines editing efficiency by first subtracting the background noise to define the expected variability in a sample. This allows the estimation of editing efficiency without the need to normalize to control samples. Following this, Chimera filters out any outliers from the noise using the Median Absolute Deviation (MAD) method and then assesses the editing efficiency of the base editor over the span of the 18-20 bp input guide sequence. High throughput sequencing data analysis, specifically frequency of single nucleotide polymorphisms (SNPs) and insertions/deletions (indels), was performed as follows: barcoded samples were demultiplexed and the demultiplexed, paired-end reads were merged using a custom Python script, which filters out any reads with mismatches in the overlapping region and keeps the higher Phred score for each overlapping base. The non-overlapping portions of the reads were then trimmed off and merged reads containing any base with a Phred score<30 were filtered out. The resulting reads were aligned using Bowtie2 and a mpileup file was generated using SAMtools.

Table 5 provides examples of chemically synthesized guides for use with CasPhi (Cas12j) and that were successfully delivered through electroporation.

The chemical modifications are noted (m=2′-O-methyl; *=phosphorothioate). Spacer region sequences are in bold. Direct repeat sequences are underlined. The ligand binding moiety sequence is italicized.

TABLE 5

| Seq | | | |
|---|---|---|---|
| ID | | ligand binding | crRNA |
| No: | Full Sequence | moiety | sequence |
| 72 | mG*mC*UUUCAA | MS2-less | Site2 gRNA4 |
| | GACUAAUAGAU | | |
| | UGCUCCUUACGA | | |
| | GGAGACAGGCU | | |
| | GGCCCGCCCCm | | |
| | G*mC*A | | |
| 73 | mG*mC*UUUCAA | MS2-less | Site2 gRNA5 |
| | GACUAAUAGAU | | |
| | UGCUCCUUACGA | | |
| | GGAGACGUGUU | | |
| | CCAGUUUCCUm | | |
| | U*mU*A | | |
| 74 | mG*mC*GCACAU | 5′ MS2 pre-cr | Site2 gRNA4 |
| | GAGGAUCACCCA | | |
| | UGUGCCUUUCA | | |
| | AGACUAAUAGA | | |
| | UUGCUCCUUACG | | |
| | AGGAGACAGGC | | |
| | UGGCCCGCCCC | | |
| | mG*mC*A | | |
| 75 | mG*mC*GCACAU | 5′ MS2 pre-cr | Site2 gRNA5 |
| | GAGGAUCACCCA | | |
| | UGUGCCUUUCA | | |
| | AGACUAAUAGA | | |
| | UUGCUCCUUACG | | |
| | AGGAGACGUGU | | |
| | UCCAGUUUCCU | | |
| | mU*mU*A | | |
| 76 | mG*mC*UUUCAA | Embedded 5′MS2 | Site2 gRNA4 |
| | GACUGCGCACAU | pre-cr | |
| | GAGGAUCACCCA | | |
| | UGUGCAAUAGA | | |
| | UUGCUCCUUACG | | |
| | AGGAGACAGGC | | |
| | UGGCCCGCCCC | | |
| | mG*mC*A | | |
| 77 | mG*mC*UUUCAA | Embedded 5′MS2 | Site2 gRNA5 |
| | GACUGCGCACAU | pre-cr | |
| | GAGGAUCACCCA | | |
| | UGUGCAAUAGA | | |
| | UUGCUCCUUACG | | |
| | AGGAGACGUGU | | |
| | UCCAGUUUCCU | | |
| | mU*mU*A | | |
| 78 | mG*mC*UUUCAA | 3′ MS2 | Site2 gRNA4 |
| | GACUAAUAGAU | | |
| | UGCUCCUUACGA | | |
| | GGAGACAGGCU | | |
| | GGCCCGCCCCG | | |
| | CAGCGCACAUGA | | |
| | GGAUCACCCAUG | | |
| | mU*mG*C | | |
| 79 | mG*mC*UUUCAA | 3′ MS2 | Site2 gRNA5 |
| | GACUAAUAGAU | | |
| | UGCUCCUUACGA | | |
| | GGAGACGUGUU | | |
| | CCAGUUUCCUU | | |
| | UAGCGCACAUGA | | |
| | GGAUCACCCAUG | | |
| | mU*mG*C | | |
| 80 | mA*mA*UAGAUU | MS2-less, no pre- | Site2 gRNA4 |
| | GCUCCUUACGAG | crRNA | |
| | GAGACAGGCUG | | |
| | GCCCGCCCCmG | | |
| | *mC*A | | |
| 81 | mA*mA*UAGAUU | MS2-less, no pre- | Site2 gRNA5 |
| | GCUCCUUACGAG | crRNA | |
| | GAGACGUGUUC | | |
| | CAGUUUCCUmU | | |
| | *mU* | | |
| 82 | mG*mC*ACAUGA | 5′ MS2, no pre- | Site2 gRNA4 |
| | GGAUCACCCAUG | crRNA | |
| | UGCAAUAGAUU | | |
| | GCUCCUUACGAG | | |
| | GAGACAGGCUG | | |
| | GCCCGCCCCmG | | |
| | *mC*A | | |
| 83 | mG*mC*ACAUGA | 5′ MS2, no pre- | Site2 gRNA5 |
| | GGAUCACCCAUG | crRNA | |
| | UGCAAUAGAUU | | |
| | GCUCCUUACGAG | | |
| | GAGACGUGUUC | | |
| | CAGUUUCCUmU | | |
| | *mU*A | | |
| 84 | mG*mC*UUUCAA | MS2-less | B2M gRNA6 |
| | GACUAAUAGAU | | |
| | UGCUCCUUACGA | | |
| | GGAGACAGGAA | | |
| | UGCCCGCCAGm | | |
| | C*mG*C | | |
| 85 | mG*mC*GCACAU | 5′ MS2 pre-cr | B2M gRNA6 |
| | GAGGAUCACCCA | | |
| | UGUGCCUUUCA | | |
| | AGACUAAUAGA | | |
| | UUGCUCCUUACG | | |
| | AGGAGACAGGA | | |
| | AUGCCCGCCAG | | |
| | mC*mG*C | | |
| 92 | mU*mC*UCGCUU | MS2-less, 2xUC on | Site2 gRNA5 |
| | UCAAGACUAAU | 5′end | |
| | AGAUUGCUCCU | | |
| | UACGAGGAGAC | | |
| | GUGUUCCAGUU | | |
| | UCCUmU*mU*A | | |
| 93 | mU*mC*UCGCGC | 5'MS2 pre-cr, 2xUC | Site2 gRNA5 |
| | ACAUGAGGAUCA | on 5′end | |
| | CCCAUGUGCCUU | | |
| | UCAAGACUAAU | | |
| | AGAUUGCUCCU | | |
| | UACGAGGAGAC | | |
| | GUGUUCCAGUU | | |
| | UCCUmU*mU*A | | |
| 94 | mG*mC*GCACAU | 5′MS2 pre-cr, 5xUC | Site2 gRNA5 |
| | GAGGAUCACCCA | internal | |
| | UGUGCUCUCUCU | | |
| | CUCCUUUCAAGA | | |
| | CUAAUAGAUUG | | |
| | CUCCUUACGAGG | | |
| | AGACGUGUUCC | | |
| | AGUUUCCUmU* | | |
| | mU*A | | |
| 95 | mU*mC*UCGCGC | 5′MS2 pre-cr, 2xUC | Site2 gRNA5 |
| | ACAUGAGGAUCA | on 5′end and 5xUC | |
| | CCCAUGUGCUCU | internal | |
| | CUCUCUCCUUUC | | |
| | AAGACUAAUAG | | |
| | AUUGCUCCUUAC | | |
| | GAGGAGACGUG | | |
| | UUCCAGUUUCC | | |
| | UmU*mU*A | | |
| 96 | mU*mC*UCCUUU | 3′MS2, 2xUC on | Site2 gRNA5 |
| | CAAGACUAAUA | 5′end | |
| | GAUUGCUCCUU | | |
| | ACGAGGAGACG | | |
| | UGUUCCAGUUU | | |
| | CCUmU*mU*AGC | | |
| | GCACAUGAGGAU | | |
| | CACCCAUGmU*m | | |
| | G*C | | |
| 97 | mC*mU*UUCAAG | 3′MS2, 5xUC | Site2 gRNA5 |
| | ACUAAUAGAUU | internal | |
| | GCUCCUUACGAG | | |
| | GAGACGUGUUC | | |
| | CAGUUUCCUmU | | |
| | *mU*AGCUCUCU | | |
| | CUCUCGCACAUG | | |
| | AGGAUCACCCAU | | |
| | GmU*mG*C | | |
| 98 | mU*mC*UCCUUU | 3′MS2, 2xUC on | Site2 gRNA5 |
| | CAAGACUAAUA | 5′end and 5xUC | |
| | GAUUGCUCCUU | internal | |
| | ACGAGGAGACG | | |
| | UGUUCCAGUUU | | |
| | CCUmU*mU*AGC | | |
| | UCUCUCUCUCGC | | |
| | ACAUGAGGAUCA | | |
| | CCCAUGmU*mG* | | |
| | C | | |
| 99 | mG*mC*UUUCAA | MS2-less | Site2_gRNA4, |
| | GACUAAUAGAU | | 20nt spacer |
| | UGCUCCUUACGA | | |
| | GGAGACAGGCU | | |
| | GGCCCGCCCCG | | |
| | CmA*mG* | | |
| 100 | mG*mC*GCACAU | 5′MS2 pre-cr | Site2_gRNA4, |
| | GAGGAUCACCCA | | 20 nt spacer |
| | UGUGCCUUUCA | | |
| | AGACUAAUAGA | | |
| | UUGCUCCUUACG | | |
| | AGGAGACAGGC | | |
| | UGGCCCGCCCC | | |
| | GCmA*mG*U | | |
| 101 | mG*mC*UUUCAA | MS2-less | Site2_gRNA5, |
| | GACUAAUAGAU | | 20 nt spacer |
| | UGCUCCUUACGA | | |
| | GGAGACGUGUU | | |
| | CCAGUUUCCUU | | |
| | UmA*mC*A | | |
| 102 | mG*mC*GCACAU | 5′MS2 pre-cr | Site2_gRNA5, |
| | GAGGAUCACCCA | | 20 nt spacer |
| | UGUGCCUUUCA | | |
| | AGACUAAUAGA | | |
| | UUGCUCCUUACG | | |
| | AGGAGACGUGU | | |
| | UCCAGUUUCCU | | |
| | UUmA*mC*A | | |
| 103 | mG*mC*UUUCAA | MS2-less | B2M_gRNA4, |
| | GACUAAUAGAU | | 20 nt spacer |
| | UGCUCCUUACGA | | |
| | GGAGACCUCUC | | |
| | CCGCUCUGCAC | | |
| | CmC*mU*C | | |
| 104 | mG*mC*GCACAU | 5′MS2 pre-cr | B2M_gRNA4, |
| | GAGGAUCACCCA | | 20 nt spacer |
| | UGUGCCUUUCA | | |
| | AGACUAAUAGA | | |
| | UUGCUCCUUACG | | |
| | AGGAGACCUCU | | |
| | CCCGCUCUGCA | | |
| | CCmC*mU*C | | |
| 105 | mG*mC*UUUCAA | MS2-less | B2M_gRNA6, |
| | GACUAAUAGAU | | 20 nt spacer |
| | UGCUCCUUACGA | | |
| | GGAGACAGGAA | | |
| | UGCCCGCCAGC | | |
| | GmC*mG*A | | |
| 106 | mG*mC*GCACAU | 5′MS2 pre-cr | B2M_gRNA6, |
| | GAGGAUCACCCA | | 20 nt spacer |
| | UGUGCCUUUCA | | |
| | AGACUAAUAGA | | |
| | UUGCUCCUUACG | | |
| | AGGAGACAGGA | | |
| | AUGCCCGCCAG | | |
| | CGmC*mG*A | | |
| 107 | GCGCACAUGAGG | 5′MS2 pre-cr | Site2 gRNA5 |
| | AUCACCCAUGUG | (unmod) | |
| | CCUUUCAAGACU | | |
| | AAUAGAUUGCU | | |
| | CCUUACGAGGA | | |
| | GACGUGUUCCA | | |
| | GUUUCCUUUAC | | |
| | A | | |
| 108 | mG*CGCACAUGA | 5′MS2 pre-cr | Site2 gRNA5 |
| | GGAUCACCCAUG | (mN* . . . N*mN) | |
| | UGCCUUUCAAG | | |
| | ACUAAUAGAUU | | |
| | GCUCCUUACGAG | | |
| | GAGACGUGUUC | | |
| | CAGUUUCCUUU | | |
| | AC*mA | | |
| 109 | mG*mC*GCACAU | 5′MS2 pre-cr | Site2 gRNA5 |
| | GAGGAUCACCCA | (mN*mN* . . . N*mN | |
| | UGUGCCUUUCA | *mN) | |
| | AGACUAAUAGA | | |
| | UUGCUCCUUACG | | |
| | AGGAGACGUGU | | |
| | UCCAGUUUCCU | | |
| | UUA*mC*mA | | |
| 110 | mG*mC*mGCACA | 5′MS2 pre-cr | Site2 gRNA5 |
| | UGAGGAUCACCC | (mN*mN*mN . . . mN | |
| | AUGUGCCUUUCA | mN*mN) | |
| | AGACUAAUAGA | | |
| | UUGCUCCUUACG | | |
| | AGGAGACGUGU | | |
| | UCCAGUUUCCU | | |
| | UUmAmC*mA | | |
| 111 | mG*mC*mG*CAC | 5′MS2 pre-cr | Site2 gRNA5 |
| | AUGAGGAUCACC | (mN*mN*mN* . . . N | |
| | CAUGUGCCUUUC | *mN*mN*mN) | |
| | AAGACUAAUAG | | |
| | AUUGCUCCUUAC | | |
| | GAGGAGACGUG | | |
| | UUCCAGUUUCC | | |
| | UUU*mA*mC*mA | | |
| 112 | mG*mC*mUUUCA | MS2-less | Site2 gRNA5 |
| | AGACUAAUAGA | (mN*mN*mN . . . mN | |
| | UUGCUCCUUACG | mN*mN) | |
| | AGGAGACGUGU | | |
| | UCCAGUUUCCU | | |
| | UUmAmC*mA | | |
| 113 | GCGCACAUGAGG | 5′MS2 pre-cr | B2M_gRNA6 |
| | AUCACCCAUGUG | (unmod) | |
| | CCUUUCAAGACU | | |
| | AAUAGAUUGCU | | |
| | CCUUACGAGGA | | |
| | GACAGGAAUGC | | |
| | CCGCCAGCGCG | | |
| | A | | |
| 114 | mG*CGCACAUGA | 5′MS2 pre-cr | B2M_gRNA6 |
| | GGAUCACCCAUG | (mN* . . . N*mN) | |
| | UGCCUUUCAAG | | |
| | ACUAAUAGAUU | | |
| | GCUCCUUACGAG | | |
| | GAGACAGGAAU | | |
| | GCCCGCCAGCG | | |
| | CG*mA | | |
| 115 | mG*mC*GCACAU | 5′MS2 pre-cr | B2M_gRNA6 |
| | GAGGAUCACCCA | (mN*mN* . . . N*mN | |
| | UGUGCCUUUCA | *mN) | |
| | AGACUAAUAGA | | |
| | UUGCUCCUUACG | | |
| | AGGAGACAGGA | | |
| | AUGCCCGCCAG | | |
| | CGC*mG*mA | | |
| 116 | mG*mC*mGCACA | 5′MS2 pre-cr | B2M_gRNA6 |
| | UGAGGAUCACCC | (mN*mN*mN . . . mN | |
| | AUGUGCCUUUCA | mN*mN) | |
| | AGACUAAUAGA | | |
| | UUGCUCCUUACG | | |
| | AGGAGACAGGA | | |
| | AUGCCCGCCAG | | |
| | CGmCmG*mA | | |
| 117 | mG*mC*mG*CAC | 5′MS2 pre-cr | B2M_gRNA6 |
| | AUGAGGAUCACC | (mN*mN*mN* . . . N | |
| | CAUGUGCCUUUC | *mN*mN*mN) | |
| | AAGACUAAUAG | | |
| | AUUGCUCCUUAC | | |
| | GAGGAGACAGG | | |
| | AAUGCCCGCCA | | |
| | GCG*mC*mG*m | | |
| | A | | |
| 118 | mG*mC*mUUUCA | MS2-less | B2M_gRNA6 |
| | AGACUAAUAGA | (mN*mN*mN . . . mN | |
| | UUGCUCCUUACG | mN*mN) | |
| | AGGAGACAGGA | | |
| | AUGCCCGCCAG | | |
| | CGmCmG*mA | | |
| 119 | mG*mC*UUUCAA | MS2-less | B2M_gRNA4 |
| | GACUAAUAGAU | | |
| | UGCUCCUUACGA | | |
| | GGAGACCUCUC | | |
| | CCGCUCUGCAm | | |
| | C*mC*C | | |
| 120 | mG*mC*GCACAU | 5′MS2 pre-cr | B2M_gRNA4 |
| | GAGGAUCACCCA | | |
| | UGUGCCUUUCA | | |
| | AGACUAAUAGA | | |
| | UUGCUCCUUACG | | |
| | AGGAGACCUCU | | |
| | CCCGCUCUGCA | | |
| | mC*mC*C | | |

### Example 6: dCasPhi Base Editing at Multiple Sites in HEK293T Cells With Chemically Synthesized and Chemically Modified Guides

HEK293T cells were electroporated with dCasPhi-2xUGI mRNA+AnoA1 mRNA+the indicated synthetic gRNAs for (FIG. 5A) B2M_gRNA4, (FIG. 5B) B2M_gRNA6 and (FIG. 5C) Site2_gRNA5. SEQ ID NOs: 119 and 120 for B2M_gRNA4; SEQ ID NOs: 84 and 85 for B2M_gRNA6; and SEQ ID NOs: 73 and 73 for Site2_gRNA5 The cells were harvested, and base editing levels were analyzed using Chimera software. The data show % C>T conversion at the indicated cytosine positions along the spacers. These data show base editing for 5′MS2 guides at a total of eleven target C residues across three spacers/genomic target sites.

### Example 7: dCasPhi Base Editing at HEK Site2 Sites in HEK293T Cells With Chemically Synthesized and Modified Synthetic Guides

HEK293T cells were electroporated with dCasPhi-2xUGI mRNA+AnoA1 mRNA+the indicated synthetic gRNAs. SEQ ID NO: 75 The dCasPhi mRNAs with different codon optimizations are noted in table 6 below. The cells were harvested, and base editing levels were analyzed using Chimera software. The data, which is summarized in FIG. 6, show % C>T conversion at the indicated cytosine positions along the spacers. These data indicate an additional DNA targeting sequence that shows base editing and that the 5′MS2 pre-cr guide may be used with multiple codon optimization of dCasPhi to carry out base editing.

TABLE 6
provides CasPhi (Cas12j) mRNAs

| SEQ ID | | |
|---|---|---|
| NO | mutation and optimization | Sequence |
| 121 | D369A, E566A & D658A | ATGGTCGACGGGAGCGGGCCGGCAGCTAAAC |
| | optimization #1 | GGGTGAAGTTGGACAGTGGTGGAATTAAACC |
| | | TACAGTTTCTCAGTTTCTTACCCCTGGTTTTAA |
| | | GCTGATAAGAAACCATAGTCGGACGGCTGGA |
| | | CTTAAGCTGAAGAATGAGGGCGAAGAGGCAT |
| | | GCAAGAAGTTCGTACGGGAGAACGAAATTCC |
| | | CAAAGATGAATGTCCAAACTTTCAAGGTGGA |
| | | CCCGCAATCGCGAACATTATAGCCAAGAGTC |
| | | GCGAATTTACCGAGTGGGAAATATATCAAAG |
| | | TTCACTGGCGATCCAAGAGGTGATTTTCACCT |
| | | TGCCGAAGGATAAGCTGCCCGAGCCTATACTC |
| | | AAGGAAGAATGGCGCGCCCAATGGTTGAGCG |
| | | AACACGGCCTCGATACGGTGCCTTACAAGGA |
| | | AGCTGCCGGACTTAATTTGATAATTAAGAACG |
| | | CGGTCAACACTTACAAAGGGGTCCAGGTGAA |
| | | AGTCGATAATAAGAATAAGAACAACCTGGCC |
| | | AAAATCAACCGCAAGAACGAAATCGCGAAAT |
| | | TGAACGGCGAACAAGAAATCAGCTTCGAAGA |
| | | GATCAAAGCCTTCGATGATAAAGGATATCTCC |
| | | TGCAAAAGCCAAGTCCGAATAAGAGCATATA |
| | | TTGCTACCAAAGCGTGTCTCCAAAGCCATTCA |
| | | TAACCTCTAAATACCATAACGTGAATCTGCCC |
| | | GAAGAATATATCGGCTACTACCGCAAGTCAA |
| | | ACGAGCCCATCGTTAGTCCCTATCAATTCGAT |
| | | AGATTGCGAATCCCAATTGGCGAACCCGGAT |
| | | ATGTACCAAAATGGCAGTATACCTTTCTGTCT |
| | | AAGAAAGAGAATAAGCGGAGAAAGCTCTCCA |
| | | AGCGGATTAAGAATGTTAGTCCTATTCTTGGG |
| | | ATAATATGCATTAAGAAAGACTGGTGCGTATT |
| | | CGATATGAGGGGCCTGCTCAGAACGAACCAC |
| | | TGGAAGAAATACCATAAACCGACAGATTCTA |
| | | TCAATGACCTCTTCGATTATTTCACTGGAGAC |
| | | CCTGTAATCGACACGAAAGCGAACGTCGTCC |
| | | GATTCAGATATAAAATGGAAAATGGCATTGTT |
| | | AATTACAAGCCGGTGCGCGAAAAGAAAGGCA |
| | | AGGAACTTTTGGAAAACATATGTGATCAAAA |
| | | TGGGAGCTGTAAGTTGGCCACTGTGGCCGTTG |
| | | GTCAAAACAACCCAGTGGCAATTGGACTGTTT |
| | | GAACTTAAGAAAGTAAATGGTGAACTTACCA |
| | | AAACCTTGATTTCACGGCATCCTACTCCGATC |
| | | GACTTTTGTAATAAAATTACGGCTTACAGGGA |
| | | GCGGTATGATAAGCTCGAATCCAGCATCAAG |
| | | TTGGATGCCATAAAGCAATTGACATCTGAGCA |
| | | AAAGATCGAAGTTGATAACTATAACAATAAT |
| | | TTTACCCCTCAAAACACTAAGCAGATAGTGTG |
| | | CAGCAAGCTCAATATCAATCCAAACGACCTTC |
| | | CTTGGGATAAAATGATTTCTGGGACTCATTTC |
| | | ATTAGCGAGAAAGCCCAAGTCAGTAATAAAT |
| | | CAGAAATATACTTCACATCTACCGATAAGGG |
| | | GAAAACTAAGGACGTAATGAAGAGCGACTAC |
| | | AAGTGGTTTCAAGACTATAAACCAAAACTGTC |
| | | AAAGGAAGTAAGGGACGCACTCAGCGATATT |
| | | GAATGGCGGCTTAGGAGAGAAAGTCTTGAAT |
| | | TTAACAAATTGAGTAAATCACGGGAACAAGA |
| | | TGCACGGCAACTGGCCAATTGGATCTCTTCCA |
| | | TGTGTGATGTTATCGGAATAGCCAACCTGGTG |
| | | AAGAAGAACAATTTCTTTGGTGGAAGCGGCA |
| | | AGAGGGAACCGGGGTGGGACAACTTCTATAA |
| | | ACCGAAGAAGGAGAATCGATGGTGGATCAAC |
| | | GCAATTCATAAAGCTCTCACAGAACTCTCTCA |
| | | AAACAAAGGGAAAAGAGTGATTCTCTTGCCA |
| | | GCAATGAGAACATCTATCACATGCCCTAAATG |
| | | TAAGTACTGTGACAGCAAGAACCGGAACGGC |
| | | GAGAAGTTCAATTGTCTGAAGTGTGGCATAG |
| | | AACTCAACGCAGCCATTGATGTTGCTACCGAA |
| | | AATCTCGCGACCGTTGCTATTACCGCGCAAAG |
| | | TATGCCTAAACCCACCTGTGAGAGGAGTGGT |
| | | GATGCCAAGAAGCCCGTACGTGCACGAAAGG |
| | | CAAAGGCGCCAGAATTTCATGACAAACTCGC |
| | | GCCCTCATACACAGTTGTCTTGCGCGAAGCTG |
| | | TTAGCGGCGGGAGCGGCGGGAGCGGGGGGAG |
| | | CACTAATCTGAGCGACATCATTGAGAAGGAG |
| | | ACTGGGAAACAGCTGGTCATTCAGGAGTCCA |
| | | TCCTGATGCTGCCTGAGGAGGTGGAGGAAGT |
| | | GATCGGCAACAAGCCAGAGTCTGACATCCTG |
| | | GTGCACACCGCCTACGACGAGTCCACAGATG |
| | | AGAATGTGATGCTGCTGACCTCTGACGCCCCC |
| | | GAGTATAAGCCTTGGGCCCTGGTCATCCAGGA |
| | | TTCTAACGGCGAGAATAAGATCAAGATGCTG |
| | | AGCGGAGGATCCGGAGGATCTGGAGGCAGCA |
| | | CCAACCTGTCTGACATCATCGAGAAGGAGAC |
| | | AGGCAAGCAGCTGGTCATCCAGGAGAGCATC |
| | | CTGATGCTGCCCGAAGAAGTCGAAGAAGTGA |
| | | TCGGAAACAAGCCTGAGAGCGATATCCTGGT |
| | | CCATACCGCCTACGACGAGAGTACCGACGAA |
| | | AATGTGATGCTGCTGACATCCGACGCCCCAGA |
| | | GTATAAGCCCTGGGCTCTGGTCATCCAGGATT |
| | | CCAACGGAGAGAACAAAATCAAAATGCTGTC |
| | | TGGCGGCTCAAAAAGAACCGCCGACGGCAGC |
| | | GAATTCGAGCCCAAGAAGAAGAGGAAAGTCT |
| | | AATGA |
| 122 | D369A, E566A, D658A | ATGGTCGACGGCAGCGGCCCCGCCGCCAAGA |
| | optimization #2 | GAGTGAAGCTGGACAGCGGCGGCATCAAGCC |
| | | CACCGTGTCTCAGTTCCTGACCCCCGGCTTCA |
| | | AGCTGATCAGAAACCACAGCAGAACTGCCGG |
| | | TCTGAAATTGAAGAACGAGGGCGAGGAGGCC |
| | | TGCAAGAAGTTCGTGAGAGAAAACGAAATCC |
| | | CCAAGGACGAGTGCCCCAACTTCCAAGGCGG |
| | | CCCCGCCATCGCCAACATCATCGCCAAGAGC |
| | | AGAGAGTTCACGGAGTGGGAGATCTATCAGA |
| | | GCAGCCTGGCCATCCAAGAGGTGATCTTCACC |
| | | CTGCCCAAGGACAAGCTGCCCGAGCCCATCCT |
| | | GAAGGAGGAGTGGAGAGCTCAGTGGCTGAGC |
| | | GAGCACGGCCTGGACACCGTGCCCTACAAGG |
| | | AGGCCGCCGGCCTGAATCTTATCATCAAGAAC |
| | | GCCGTGAACACCTACAAGGGCGTGCAAGTGA |
| | | AGGTGGACAACAAGAACAAGAACAACCTGGC |
| | | CAAGATCAACAGAAAGAACGAGATCGCCAAG |
| | | CTGAATGGCGAACAAGAGATCAGCTTCGAGG |
| | | AGATCAAGGCCTTCGACGACAAGGGCTACCT |
| | | GCTGCAGAAGCCTAGCCCCAATAAGAGCATC |
| | | TACTGCTATCAGAGCGTGAGCCCCAAGCCCTT |
| | | CATCACAAGCAAGTACCACAACGTGAACCTG |
| | | CCCGAGGAGTACATCGGCTACTACAGAAAGA |
| | | GCAACGAGCCCATCGTGAGCCCCTATCAGTTC |
| | | GACAGACTGAGAATCCCCATCGGCGAGCCCG |
| | | GCTACGTGCCCAAGTGGCAGTACACCTTCCTG |
| | | AGCAAGAAGGAAAACAAAAGAAGAAAGCTC |
| | | AGCAAGAGAATCAAGAACGTGAGCCCCATCC |
| | | TGGGCATCATCTGCATCAAGAAGGACTGGTG |
| | | CGTGTTCGACATGAGAGGCCTGCTGAGAACC |
| | | AACCACTGGAAGAAGTACCACAAGCCCACCG |
| | | ACAGCATCAACGACCTGTTCGACTATTTCACC |
| | | GGCGACCCCGTGATCGACACCAAGGCCAACG |
| | | TGGTGAGATTCAGATACAAGATGGAGAACGG |
| | | CATCGTGAACTACAAGCCCGTTCGCGAGAAA |
| | | AAGGGCAAGGAGCTGCTGGAGAACATCTGCG |
| | | ATCAGAACGGCAGCTGCAAGCTGGCGACTGT |
| | | GGCCGTGGGGCAGAACAATCCCGTGGCCATC |
| | | GGCCTGTTCGAGCTGAAGAAGGTAAACGGCG |
| | | AGCTGACCAAGACCCTGATCAGCAGACACCC |
| | | CACCCCCATCGACTTCTGCAACAAGATCACCG |
| | | CCTACAGAGAGAGATACGACAAGCTGGAGTC |
| | | TAGCATCAAGCTGGACGCCATCAAGCAGCTG |
| | | ACAAGCGAGCAGAAGATCGAGGTGGACAACT |
| | | ACAACAACAACTTCACCCCTCAGAACACCAA |
| | | GCAGATCGTGTGCAGCAAGCTGAACATCAAC |
| | | CCCAACGACCTGCCCTGGGACAAGATGATCA |
| | | GCGGCACCCACTTCATTTCCGAGAAGGCCCAA |
| | | GTGAGCAACAAGAGCGAGATCTACTTCACAA |
| | | GCACCGACAAGGGAAAGACCAAAGACGTGAT |
| | | GAAGAGCGACTATAAGTGGTTCCAAGACTAC |
| | | AAACCCAAACTAAGCAAAGAGGTGCGGGACG |
| | | CCCTGAGCGACATCGAGTGGAGACTGAGAAG |
| | | AGAGAGCCTGGAGTTCAACAAATTATCGAAA |
| | | TCTCGGGAGCAAGACGCTAGACAGCTGGCCA |
| | | ACTGGATCAGCAGCATGTGCGACGTGATCGG |
| | | CATCGCCAACCTGGTGAAGAAGAACAACTTC |
| | | TTCGGCGGCAGCGGCAAGAGAGAGCCCGGCT |
| | | GGGACAACTTCTACAAGCCCAAGAAAGAGAA |
| | | CAGATGGTGGATCAACGCCATCCACAAGGCC |
| | | CTGACCGAGCTGTCTCAGAACAAGGGCAAGA |
| | | GAGTGATCCTGCTGCCCGCCATGAGAACAAG |
| | | CATCACCTGCCCCAAGTGCAAGTACTGCGACA |
| | | GCAAGAACAGAAACGGCGAGAAGTTCAACTG |
| | | CCTGAAGTGCGGCATCGAGCTGAACGCCGCC |
| | | ATCGACGTGGCCACCGAGAACCTAGCTACCG |
| | | TGGCCATCACCGCTCAGAGCATGCCCAAGCCC |
| | | ACCTGCGAGAGAAGCGGCGACGCCAAGAAAC |
| | | CCGTCAGAGCTCGCAAGGCCAAGGCCCCCGA |
| | | GTTCCATGACAAGCTGGCCCCAAGCTACACCG |
| | | TGGTGCTGAGAGAGGCCGTGAGCGGCGGGAG |
| | | CGGCGGGAGCGGGGGGAGCACTAATCTGAGC |
| | | GACATCATTGAGAAGGAGACTGGGAAACAGC |
| | | TGGTCATTCAGGAGTCCATCCTGATGCTGCCT |
| | | GAGGAGGTGGAGGAAGTGATCGGCAACAAGC |
| | | CAGAGTCTGACATCCTGGTGCACACCGCCTAC |
| | | GACGAGTCCACAGATGAGAATGTGATGCTGC |
| | | TGACCTCTGACGCCCCCGAGTATAAGCCTTGG |
| | | GCCCTGGTCATCCAGGATTCTAACGGCGAGA |
| | | ATAAGATCAAGATGCTGAGCGGAGGATCCGG |
| | | AGGATCTGGAGGCAGCACCAACCTGTCTGAC |
| | | ATCATCGAGAAGGAGACAGGCAAGCAGCTGG |
| | | TCATCCAGGAGAGCATCCTGATGCTGCCCGAA |
| | | GAAGTCGAAGAAGTGATCGGAAACAAGCCTG |
| | | AGAGCGATATCCTGGTCCATACCGCCTACGAC |
| | | GAGAGTACCGACGAAAATGTGATGCTGCTGA |
| | | CATCCGACGCCCCAGAGTATAAGCCCTGGGCT |
| | | CTGGTCATCCAGGATTCCAACGGAGAGAACA |
| | | AAATCAAAATGCTGTCTGGCGGCTCAAAAAG |
| | | AACCGCCGACGGCAGCGAATTCGAGCCCAAG |
| | | AAGAAGAGGAAAGTCTAATGA |

### Example 8: The Effects of Synthetic Guide Chemical Modifications on Base Editing Levels

Guide RNAs for Site2 gRNA5, SEQ ID NOs: 107, 108, 109, 110, 111, and 112 (table 5), and B2M gRNA6 SEQ ID NO: 113, 114, 115, 86, 116, 117 and 118 (table 5), were synthesized with different combinations of chemical modifications on 5′ and 3′ ends (see table of sequences for details). HEK293T cells were electroporated with dCasPhi-2xUGI mRNA+AnoA1 mRNA+synthetic gRNAs. Base editing levels were measured by Chimera or NGS and compared to the gRNA with mN*mN* . . . mN*mN*N modifications. Data show % C>T conversion at the indicated cytosines along the spacer. The results are shown in FIG. 7A and 7B, respectively. The mN*mN* . . . mN*mN*N guides were chemically synthesized in a different batch from the rest of the guides

These data indicate that there are several chemical modification patterns that offer significantly improved base editing levels over unmodified chemically synthesized guides, e.g., a single 2′-O-methyl modification and phosphorothioate linkage at both the 5′ and 3′ end as well as additional incorporations of two or three 2′-Omethyl modifications and phosphorothioate linkages.

### Example 9: Assessment of the Effects of Linkers in gRNA Sequences on Base Editing Levels

Guide RNAs were designed with (UC) linkers on the 5′ end, and/or between the MS2 and the spacer, and/or on the 3′ end of the guide. SEQ ID NO: 73, 75, 79, 92, 93, 94, 95, 96, 97, and 98 (table 5). RNAs were synthesized by the same method with the same chemical modifications. HEK293T cells were electroporated with dCasPhi-2xUGI mRNA+AnoA1 deaminase mRNA+synthetic gRNA. Base editing levels were measured by Chimera or NGS and compared to the gRNA without linkers. Data show the levels of C>T conversion at the indicated cytosines along the spacer sequence.

These data, summarized in FIG. 8A suggest that addition of additional UC linkers outside of the current GC linker allow for similar levels base editing levels, compared to having no additional linkers. FIG. 8B (SEQ ID NO: 29) shows a template gRNA with chemical modifications in the absence of a ligand binding moiety. FIGS. 8C to 8K show templates with different modification patterns and one or two linkers. (SEQ ID NO: 30-32 and 86-91)

### Example 10: CasPhi Aptamer-Recruitment Base Editing in T Lymphocytes

CD3+ T Cell Isolation from Fresh Blood Sources and Culturing: PBMCs were isolated from blood sources (e.g., CPD Blood bags, apheresis cones, leukopaks, etc.) by layering on Lymphoprep using SepMap columns (STEMCELL Technologies). Then total CD3+ T cells were isolated using negative selection with the EasySep Human T Cell Isolation Kit (STEMCELL Technologies). T Cells were checked by flow cytometry and then cultured in Immunocult XT media (STEMCELL Technologies) with 1'3 Penicillin/Streptomycin (Thermofisher) at 37 C and 5% CO2.

T Cell Electroporation: After 48-72 post-activation T cells were electroporated with using the Neon Electroporator (Thermofisher). Neon Electroporator conditions were 1600v/10 ms/3 pulses with a 10 ul tip with 250k cells, combined total mRNA concentration of 100 ng/ul, for both the Deaminase-MCP and nCasPhi-UGI-UGI (synthesized by Trilink), and the crRNA was a final concentration of 2 uM. Post-electroporation cells were transferred to Immunocult XT media with 100U IL-2, 100U IL-7 and 100U IL-15 (STEMCELL Technologies) and cultured at 37 C and 5% CO2 for 48-72 hours.

CD3+ T Cell Activation: T cells were activated by using 1:1 bead:cell ratio of Dynabeads Human T Activator CD3/CD28 beads (Thermofisher) cultured in Immunocult XT media (STEMCELL Technologies) in the presence of 100U/ml IL-2 (STEMCELL Technologies) and 1× Penicillin/Streptomycin (Thermofisher) at 37 C and 5% CO2 for 48-72 hours. Post-activation, beads were removed by placement on a magnet and the transfer of the cells back into culture.

Genomic DNA Analysis: Genomic DNA was released from lysed cells 48-72 hours post-electroporation. Locus of interest were amplified by PCR and products then sent for Sanger sequencing. Data was analyzed by Chimera.

Synthetic crRNA Sequence (without aptamer) against B2M locus: SEQ ID NO: 84 from Table 5.

Synthetic crRNA Sequence (with 1xMS2 aptamer) against B2M locus: SEQ ID NO: 85 from Table 5.

T lymphocytes were stimulated and then electroporated in the presence of a different aptamer designs with the same deaminase. The data, summarized in FIG. 9, shows that traceless Type V family can be utilized with specific aptamer-based recruitment base editing in T lymphocytes.

### Example 11: Electroporation of mRNA and Synthetic Guides For dCas12a-UGI Base Editing

mRNA Preparation:

Messenger mRNA are prepared from DNA vectors carrying the T7 promoter and the coding sequences for dCas12a-UGI and AnoA1 following the standard protocols for mRNA in vitro transcription.

RNA Synthesis:

All crRNA were synthesized by Horizon Discovery using either 2′-acetoxy ethyl orthoester (2′-ACE) or 2′-tert-butyldimethylsilyl (2′-TBDMS) protection chemistries or by Agilent. Chemical modifications were included where noted. RNA oligos were 2′-depotected/desalted and purified by either high-performance liquid chromatography (HPLC) or polyacrylamide gel electrophoresis (PAGE). Oligos were resuspended in 10 mM Tris pH7.5 buffer prior to electroporation.

HEK 293T cells (ATCC, #CRL-11268) were electroporated using the Invitrogen™ Neon™ Transfection System, 10 uL Kit. A mixture of 50,000 cells, 1 μg of mRNA, and 6 μM of synthetic crRNA were electroporated at 1150V for 20 ms and for 2 pulses. mRNA was mixed at a 3:1 molar ratio of dCas12a-2xUGI to AnoA1. Cells were plated in a 96-well plate with full serum growth media and harvested after 72 hours for further processing.

Cell Processing

Cells may be lysed in 100 μL of a buffer containing proteinase K (Thermo Scientific, #FEREO0492), RNase A (Thermo Scientific, #FEREN0531), and Phusion HF buffer (Thermo Scientific, #F-518L) for 30 min at 56° C., followed by a 5 min heat inactivation at 95° C. This cell lysate may be used to generate PCR amplicons spanning the region containing the base editing site(s). PCR amplicons between 400-1000 bp in length may be sequenced by Sanger sequencing.

Editing Analysis

Base editing efficiencies may be calculated using the Chimera analysis tool, an adaptation of the open source tool BEAT. Chimera determines editing efficiency by first subtracting the background noise to define the expected variability in a sample. This allows the estimation of editing efficiency without the need to normalize to control samples. Following this, Chimera filters out any outliers from the noise using the Median Absolute Deviation (MAD) method and then assesses the editing efficiency of the base editor over the span of the 23 nt input guide sequence.

Table 7 provides chemically synthesized gRNA for use with Cas 12a and the gRNA used in this example Chemically modified synthetic guides SEQ ID NO: 142 -144 demonstrated desirable levels of base editing. Spacer region sequences are in bold. Direct repeat sequences are underlined. The ligand binding moiety sequence is italicized.

TABLE 7

| | | ligand | |
|---|---|---|---|
| Seq ID | | binding | |
| NO | Full Sequence | moiety | crRNA |
| 142 | mG*mC*GCACAUGAGGAUCACCCAUGUGCUAAUUUCU | 5′ MS2 | Site2_guide3 |
| | ACUAAGUGUAGAUCAGCCCGCTGGCCCTGTAAAmG | | |
| | *mG*A | | |
| 143 | mG*mC*GCACAUGAGGAUCACCCAUGUGCGUCAAAAG | 5′ pre- | Site2_guide3 |
| | ACUUUUUAAUAAUUUCUACUAAGUGUAGAUCAGCC | MS2 | |
| | CGCTGGCCCTGTAAAmG*mG*A | | |
| 144 | mU*mA*AUUUCUACUAAGUGUAGAUCAGCCCGCTG | 3′ MS2 | Site2_guide3 |
| | GCCCTGTAAAGGAGCGCACAUGAGGAUCACCCAUGm | | |
| | U*mG*C | | |
| 145 | mG*mU*CAAAAGACUUUUUAAUAAUUUCUACUAAGU | 3′ pre- | Site2_guide3 |
| | GUAGAUCAGCCCGCTGGCCCTGTAAAGGAGCGCAC | MS2 | |
| | AUGAGGAUCACCCAUGmU*mG*C | | |
| 146 | mG*mU*CAAAAGACUUUUUAAUAAUUUCUACUAAGU | MS2-less | Site2_guide3 |
| | GUAGAUCAGCCCGCTGGCCCTGTAAAmG*mG*A | | |

### Example 12: dCas12a Base Editing at HEK Site2 With Chemically Modified Guides

HEK293T cells were electroporated with dCas12a-2xUGI mRNA+AnoA1 mRNA+the indicated synthetic gRNAs. The cells were harvested, and base editing levels were analyzed by NGS. The data, summarized in FIG. 10, show % C>T conversion at the indicated cytosine positions along the spacers. This data demonstrates that Cas12a and corresponding guides are effective at base editing.

### Example 13: Transfection of Plasmid Components For dCas12i2 Base Editing

The coding sequence for a deactivated version of Cas12i2 and 2xUGI fusion (dCas12i2-UGI) was obtained and cloned into an expression vector under the control of the mouse CMV promoter in a T2A polycistronic cassette with a red fluorescent protein-puromycin fusion. The coding sequence for MS2 coat protein lizard Anolis Apobec fusion (AnoA1) was obtained and cloned into an expression vector under control of the mouse CMV promoter. The coding sequence for MS2 coat protein human APOBEC3A (hA3A) fusion (MCP-hA3A) was obtained and cloned into an expression vector under control of the mouse CMV promoter. The sequence for crRNA containing the MS2 ligand binding moiety and unique spacer regions were cloned into an expression vector under control of the hU6 promoter.

HEK 293T cells (ATCC, #CRL-11268) were seeded at 20,000 cells per well in a 96-well plate one day prior to transfection. Cells were co-transfected using DharmaFECT Duo Transfection Reagent (Horizon Discovery, #T-2010) and 75-200 ng dCas12i2-UGI plasmid, 75-100 ng AnoA1 or hA3Aplasmid, and 50-100 ng crRNA plasmid. The plasmid crRNA consisted of a direct repeat length of 31 nucleotides, different spacer sequences of 31 nucleotides targeting transcripts within Site2 or B2M gene targets, and have the MS2 ligand binding moiety at the 5′ terminus, the 3′ terminus, internally not at either the 5′ or 3′ terminus, or combinations therein. Sequences are provided in Table 8 below.

Cells were grown for 72 hours post-transfection and harvested for further processing.

Cell Processing

Cells were lysed in 100 μL of a buffer containing proteinase K (Thermo Scientific, #FEREO0492), RNase A (Thermo Scientific, #FEREN0531), and Phusion HF buffer (Thermo Scientific, #F-518L) for 30 min at 56° C., followed by a 5 min heat inactivation at 95° C. This cell lysate was used to generate PCR amplicons spanning the region containing the base editing site(s). PCR amplicons between 400-1000 bp in length were sequenced by Sanger sequencing.

Editing Analysis

Base editing efficiencies were calculated using the Chimera analysis tool, an adaptation of the open source tool BEAT. Chimera determines editing efficiency by first subtracting the background noise to define the expected variability in a sample. This allows the estimation of editing efficiency without the need to normalize to control samples. Following this, Chimera filters out any outliers from the noise using the Median Absolute Deviation (MAD) method and then assesses the editing efficiency of the base editor over the span of the 31 bp input guide sequence.

TABLE 8
provides guides generated by plasmid for use
with Cas1212::

| | | ligand binding | crRNA | Type V |
|---|---|---|---|---|
| Seq ID NO | Full Sequence | moiety | sequence | enzyme used |
| 125 | GCGCACATGA | 5′ MS2 | Site2 guide 7 | Cas1212 |
| | GGATCACCCA | | | |
| | TGTGCAGAA | | | |
| | ATCCGTCTTT | | | |
| | CATTGACGG | | | |
| | ACAGATGG | | | |
| | GGCTGGAC | | | |
| | AATTTTTCC | | | |
| | CCCTTT | | | |
| 126 | AGAAATCCG | 3′ MS2 | Site2 guide 7 | Cas12i2 |
| | TCTTTCATTG | | | |
| | ACGGACAGA | | | |
| | TGGGGCTG | | | |
| | GACAATTTT | | | |
| | TCCCCCTTT | | | |
| | GCGCACATGA | | | |
| | GGATCACCCA | | | |
| | TGTGC | | | |
| 127 | AGAAATCCG | MS2-Less | Site2 guide 7 | Cas12i2 |
| | TCTTTCATTG | | | |
| | ACGGACAGA | | | |
| | TGGGGCTG | | | |
| | GACAATTTT | | | |
| | TCCCCCTTT | | | |
| 128 | GCGCACATGA | 5′ MS2 | Site2 guide 8 | Cas12i2 |
| | GGATCACCCA | | | |
| | TGTGCAGAA | | | |
| | ATCCGTCTTT | | | |
| | CATTGACGG | | | |
| | CAGTTTCCT | | | |
| | TTACAGGGC | | | |
| | CAGCGGGC | | | |
| | TGGAA | | | |
| 129 | AGAAATCCG | 3′ MS2 | Site2 guide 8 | Cas12i2 |
| | TCTTTCATTG | | | |
| | ACGGCAGTT | | | |
| | TCCTTTACA | | | |
| | GGGCCAGC | | | |
| | GGGCTGGA | | | |
| | AGCGCACAT | | | |
| | GAGGATCACC | | | |
| | CATGTGC | | | |
| 130 | AGAAATCCG | MS2-Less | Site2 guide 8 | Cas12i2 |
| | TCTTTCATTG | | | |
| | ACGGCAGTT | | | |
| | TCCTTTACA | | | |
| | GGGCCAGC | | | |
| | GGGCTGGA | | | |
| | A | | | |
| 131 | GCGCACATGA | 5′ MS2 | B2M guide 6 | Cas12i2 |
| | GGATCACCCA | | | |
| | TGTGCAGAA | | | |
| | ATCCGTCTTT | | | |
| | CATTGACGG | | | |
| | AGGAATGC | | | |
| | CCGCCAGC | | | |
| | GCGACGCC | | | |
| | TCCACTT | | | |
| 132 | AGAAATCCG | 3′ MS2 | B2M guide 6 | Cas12i2 |
| | TCTTTCATTG | | | |
| | ACGGAGGAA | | | |
| | TGCCCGCCA | | | |
| | GCGCGACG | | | |
| | CCTCCACTT | | | |
| | GCGCACATGA | | | |
| | GGATCACCCA | | | |
| | TGTGC | | | |
| 133 | AGAAATCCG | MS2-Less | B2M guide 6 | Cas1212 |
| | TCTTTCATTG | | | |
| | ACGGAGGAA | | | |
| | TGCCCGCCA | | | |
| | GCGCGACG | | | |
| | CCTCCACTT | | | |

### Example 14: Transfection of Plasmid Components For dCas12i2 Base Editing at HEK Site2 With Multiple Deaminases

SEQ ID NO: 128, 129, and 130 from table 8 were used in this example

HEK293T cells were transfected with plasmids for: dCas12i2-2xUGI+AnoA1 or hA3A+the indicated gRNAs. The cells were harvested and base editing levels analyzed by Chimera. The data, summarized in FIG. 11, show % C>T conversion at the indicated cytosine positions along the spacers. This data demonstrates that Cas12i2 and corresponding guides are effective at base editing with either of the AnoA1 (FIG. 11A) or hA3A (FIG. 11B) deaminases and either a 5′ MS2 or 3′ MS2 aptamer location within the gRNA.

1. A gRNA-ligand binding complex, wherein the gRNA-ligand binding complex comprises:
a. a gRNA, wherein the gRNA is 35 to 60 nucleotides long and the gRNA has a crRNA sequence, wherein the crRNA sequence is 35 to 60 nucleotides long and the crRNA sequence comprises a Cas association region, wherein the Cas association region is 14 to 37 nucleotides long and a targeting region, wherein the targeting region is 14 to 37 nucleotides long and the Cas association region is capable of retaining association with an RNA binding domain of a Type V Cas protein in the absence of a tracrRNA; and
b. a ligand binding moiety, wherein the ligand binding moiety is either (i) directly bound to the gRNA, or (ii) bound to the gRNA through a linker.

2. The gRNA-ligand binding complex of claim 1, wherein at least one of the gRNA and the ligand binding moiety comprises at least one modification, wherein said at least one modification imparts resistance to an active nuclease domain of the Type V Cas protein relative to a gRNA-ligand binding complex that lacks said at least one modification.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. The gRNA-ligand binding complex of claim 1, wherein the gRNA has a 3′ end and the ligand binding moiety is directly bound to the 3′ end of the gRNA.

19. The gRNA-ligand binding complex of claim 1, wherein the gRNA has a 5′ end and the ligand binding moiety is directly bound to the 5′ end of the gRNA.

20. (canceled)

21. (canceled)

22. The gRNA-ligand binding complex of claim 1, wherein the ligand binding moiety is located between the Cas association region and the targeting region.

23. The gRNA-ligand binding complex of claim 22, wherein the ligand binding moiety forms a stem-loop complex.

24. The gRNA-ligand binding complex of claim 1, wherein the gRNA- ligand binding complex comprises the linker, and the ligand binding moiety is bound to the gRNA through the linker.

25. (canceled)

26. (canceled)

27. The gRNA-ligand binding complex of claim 24, wherein the gRNA has a 3′ end and the linker is bound to the 3′ end of the gRNA.

28. The gRNA-ligand binding complex of claim 24, wherein the gRNA has a 5′ end and the linker is bound to the 5′ end of gRNA.

29. (canceled)

30. (canceled)

31. (canceled)

32. (canceled)

33. (canceled)

34. The gRNA-ligand binding complex of claim 24, wherein the linker is a first linker and the gRNA-ligand binding complex further comprises a second linker, wherein the ligand binding moiety is located between the first linker and the second linker and each of the first linker and the second linker is immediately adjacent to the ligand binding moiety.

35. (canceled)

36. (canceled)

37. (canceled)

38. (canceled)

39. (canceled)

40. (canceled)

41. (canceled)

42. (canceled)

43. (canceled)

44. The gRNA-ligand binding complex of claim 1, wherein the ligand binding moiety is able to associate with a ligand from the group consisting of: MS2, Ku, PP7, SfMu, Sm7, Tat, Glutathione S-transferase (GST), CSY4, Qbeta, COM, pumilio, Anti-His Tag (6H7), lambda N22plus, SNAP-Tag, a lectin, and PDGF beta-chain.

45. (canceled)

46. (canceled)

47. (canceled)

48. (canceled)

49. (canceled)

50. (canceled)

51. A base editing complex comprising:
a. the gRNA-ligand binding complex of claim 1; and
b. a Type V Cas protein, wherein the Cas association region of the gRNA-ligand binding complex is associated with the Type V Cas protein.

52. (canceled)

53. The base editing complex of claim 51, wherein the Type V Cas protein comprises an active RuvC domain.

54. The base editing complex of claim 51, wherein the Type V Cas protein comprises a deactivated RuvC domain.

55. (canceled)

56. The base editing complex of any claim 51 further comprising an effector, wherein the effector is attached to a ligand and the ligand is capable of associating with the ligand binding moiety.

57. The base editing complex of claim 56, wherein the effector is a deaminase.

58. (canceled)

59. (canceled)

60. (canceled)

61. (canceled)

62. (canceled)

63. (canceled)

64. A method for base editing comprising exposing the base editing complex of claim 51 to double-stranded DNA.

65. (canceled)

66. (canceled)

67. (canceled)

68. (canceled)

69. (canceled)

70. (canceled)

71. (canceled)

72. A method of editing DNA in a cell, said method comprising exposing the base editing complex of claim 51 to the cell.

73. A population of genetically edited cells obtained according to the method claim 72.

74. (canceled)

75. A method of treating a subject, said method comprising the method of claim 72, wherein said exposing takes place outside of a subject and after said exposing, infusing the cell into the subject.

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)


SEQUENCE LISTING (147 sequences)

SEQ ID NO: 1 - RNA, 22 nt, Artificial Sequence
  Synthetic
  misc_feature (22)..(22): N is  A, C, G. or U
uuaauuucuacucuuguagaun

SEQ ID NO: 2 - RNA, 19 nt, Artificial Sequence
  Synthetic
  misc_feature (19)..(19): N is  A, C, G. or U
ugcucgauuagucgacacn

SEQ ID NO: 3 - RNA, 37 nt, Artificial Sequence
  Synthetic
  misc_feature (37)..(37): N is   A, C, G. or U
ggagagaucucaaacgauugcucgauuagucgagacn

SEQ ID NO: 4 - RNA, 37 nt, Artificial Sequence
  Synthetic
  misc_feature (37)..(37): N  is  A, C, G. or U
gucggaacgcucaacgauugccccucacgaggggacn

SEQ ID NO: 5 - RNA, 37 nt, Artificial Sequence
  Synthetic
  misc_feature (37)..(37): N is  A, C, G. or U
accaaaacgacuauugauugcccaguacgcugggacn

SEQ ID NO: 6 - RNA, 37 nt, Artificial Sequence
  Synthetics
  misc_feature (37)..(37): N is  A, C, G. or U
auggcaacagacucucauugcgcgguacgccgcgacn

SEQ ID NO: 7 - RNA, 38 nt, Artificial Sequence
  Synthetic
  misc_feature (38)..(38): N is A, C, G. or U
gucccaacgaauugggcaaucaaaaaggauuggauccn

SEQ ID NO: 8 - RNA, 37 nt, Artificial Sequence
  Synthetic
  misc_feature (37)..(37): N is 16 to 30 nucleotides, each is  A, C, G. or U
ccugcgaaaccuuuugauugcucaguacgcugagacn

SEQ ID NO: 9 - RNA, 37 nt, Artificial Sequence
  Synthetic
  misc_feature (37)..(37): N is  A, C, G. or U
cuuucaagacuaauagauugcuccuuacgaggagacn

SEQ ID NO: 10 - RNA, 37 nt, Artificial Sequence
  Synthetic
  misc_feature (37)..(37): N is A, C, G, or U
guagaagaccucgcugauugcucggugcgccgagacn

SEQ ID NO: 11 - RNA, 21 nt, Artificial Sequence
  Synthetic
  misc_feature (21)..(21): N is  A, C, G. or U
uaauuucuacucuuguagaun

SEQ ID NO: 12 - RNA, 22 nt, Artificial Sequence
  Synthetic
  misc_feature (22)..(22): N is  A, C, G. or U
uaauuucuacuaaguguagaun

SEQ ID NO: 13 - RNA, 19 nt, Artificial Sequence
  MS2 Phage operator stem loop
acaugaggaucacccaugu

SEQ ID NO: 14 - RNA, 66 nt, Artificial Sequence
  Telomerase binding motif
uucuugucguacuuauagaucgcuacguuauuucaauuuugaaaaucugaguccugggagugcgga

SEQ ID NO: 15 - protein, 609 aa, Artificial Sequence
  Ku heterodimer
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 16 - protein, 485 aa, Artificial Sequence
  Ku heterodimer
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 17 - RNA, 10 nt, Artificial Sequence
  Sm consensus site
aauuuuugga

SEQ ID NO: 18 - protein, 83 aa, Artificial Sequence
  Monomeric Sm - like protein
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 19 - RNA, 19 nt, Artificial Sequence
  MS2 phage operator stem loop
acaugaggaucacccaugu

SEQ ID NO: 20 - protein, 117 aa, Artificial Sequence
  MS2 coat protein
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 21 - RNA, 26 nt, Artificial Sequence
  PP7 phage operator stem loop
auaaggaguuuauauggaaacccuua

SEQ ID NO: 22 - protein, 128 aa, Artificial Sequence
  PP7 Phage Protein
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 23 - RNA, 20 nt, Synthliboramphus antiquus
gcugaaugccugcgagcauc

SEQ ID NO: 24 - protein, 62 aa, Artificial Sequence
  Lambda N22 Plus Protein
MKSIRCKNCNKLLFKADSFDHIEIRCPRCKRHIIMLNACEHPTEKHCGKREKITHSDETVRY

SEQ ID NO: 25 - RNA, 15 nt, Artificial Sequence
  Box B Aptamer
gcccugaagaagggc

SEQ ID NO: 26 - protein, 22 aa, Artificial Sequence
  Lambda N22 plus protein
MNARTRRRERRAEKQAQWKAAN

SEQ ID NO: 27 - RNA, 16 nt, Artificial Sequence
  Csy4 binding motif
cugccguauaggcagc

SEQ ID NO: 28 - protein, 187 aa, Artificial Sequence
  Csy4[H29A]
MDHYLDIRLRPDPEFPPAQLMSVLFGKLAQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWF

SEQ ID NO: 29 - RNA, 56 nt, Artificial Sequence
  Synethic
  misc_feature (1)..(2): 2′ -O-methyl
  N_region (39)..(56): N is A, C, G or U
  misc_feature (39)..(53): n is a, c, g, or u
  misc_feature (54)..(55): 2′ -O-methyl
  misc_feature (56)..(56): n is a, c, g, or u
cgcuuucaagacuaauagauugcuccuuacgaggagacnnnnnnnnnnnnnnnnnn

SEQ ID NO: 30 - RNA, 78 nt, Artificial Sequence
  Synthetic
  misc_feature (1)..(2): 2′-O-methyl gorup
  N_region (61)..(78): N is A, C, G. or U
  misc_feature (61)..(75): n is a, c, g, or u
  misc_feature (76)..(77): 2′-O-methyl gorup
  misc_feature (78)..(78): n is a, c, g, or u
gcgcacaugaggaucacccaugugcuuucaagacuaauagauugcuccuuacgaggagacnnnnnnnnnnnnnnnnnn

SEQ ID NO: 31 - RNA, 81 nt, Artificial Sequence
  Synthetic
  misc_feature (1)..(2): 2′-O-methyl
  N_region (38)..(55): N is A, C, G. or U
  misc_feature (38)..(55): n is a, c, g, or u
  misc_feature (79)..(80): 2′-O-methyl
gcuuucaagacuaauagauugcuccuuacgaggagacnnnnnnnnnnnnnnnnnngcgcacaugaggaucaacccaugugc

SEQ ID NO: 32 - RNA, 59 nt, Artificial Sequence
  Synthetic
  misc_feature (1)..(2): 2′-O-methyl
  N_region (42)..(59): N is A, C, G, or U
  misc_feature (42)..(56): n is a, c, g, or u
  misc_feature (57)..(58): 2′-O-methyl
  misc_feature (59)..(59): n is a, c, g, or u
ucucgcuuucaagacuaagagauugcuccuuacgaggagacnnnnnnnnnnnnnnnnnn

SEQ ID NO: 33 - DNA, 687 bp, Unknown
  Rat APO
atgtcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaag

SEQ ID NO: 34 - DNA, 594 bp, Unknown
  GgAID
atggattctctgctgatgaagaggaagctgtttctgtacaattttaagaatctgaggtgggccaagggcagaagggagacctatctgtgctacgtggtgaagagaagggacagcgccaccagctgcagcctcgatttcggctatctgaggaacaagatgggctgtcacgtggaggtgctgtttctgagatacatctccgcttgggatctggatcccggcagatgctatagaatcacatggttcaccagctggagcccttgttacgactgtgctagacatgtggccgactttctgagggcctatcccaatctgacactgagaatcttcaccgctagactgtacttctgcgaggacagaaaggctgagcccgagggactgagaaggctgcacagagccggcgcccagatcgccatcatgacctttaaggactttttctattgctggaacaccttcgtggagaatagagagaagaccttcaaggcttgggagggactgcacgagaactccgtgcatctgtctagaaagctgaggagaattctgctgcctctgtatgaggtggacgatctgagagatgccttcaagaccctcggactg

SEQ ID NO: 35 - DNA, 54 bp, Artificial Sequence
  Synthetic
ctttcaagactaatagattgctccttacgaggagacaggctggcccgccccgca

SEQ ID NO: 36 - DNA, 54 bp, Artificial Sequence
  Synthetic
ctttcaagactaatagattgctccttacgaggagacgtgttccagtttccttta

SEQ ID NO: 37 - DNA, 78 bp, Artificial Sequence
  Synthetic
cgcacatgaggatcacccatgtgcctttcaagactaatagattgctccttacgaggagacaggctggcccgccccgca

SEQ ID NO: 38 - DNA, 78 bp, Artificial Sequence
  Synthetic
cgcacatgaggatcacccatgtgcctttcaagactaatagattgctccttacgaggagacgtgttccagtttccttta

SEQ ID NO: 39 - DNA, 80 bp, Artificial Sequence
  Synthetic
ctttcaagactgcgcacatgaggatcacccatgtgcaatagattgctccttacgaggagacaggctggcccgccccgcac

SEQ ID NO: 40 - DNA, 79 bp, Artificial Sequence
  Synthetic
ctttcaagactgcgcacatgaggatcacccatgtgcaatagattgctccttacgaggagacgtgttccagtttccttta

SEQ ID NO: 41 - DNA, 77 bp, Artificial Sequence
  Synthetic
ctttcaagactaatagattgctccttacaacatgaggatcacccatgttgcgaggagacaggctggcccgccccgca

SEQ ID NO: 42 - DNA, 77 bp, Artificial Sequence
  Synthetic
ctttcaagactaatagattgctccttacaacatgaggatcacccatgttgcgaggagacgtgttccagtttccttta

SEQ ID NO: 43 - DNA, 79 bp, Artificial Sequence
  Synthetic
ctttcaagactaatagattgctccttaacaacatgaggatcacccatgttgccgaggagacaggctggcccgccccgca

SEQ ID NO: 44 - DNA, 79 bp, Artificial Sequence
  Synthetic
ctttcaagactaatagattgctccttaacaacatgaggatcacccatgttgccgaggagacgtgttccagtttccttta

SEQ ID NO: 45 - DNA, 79 bp, Artificial Sequence
  Synthetic
ctttcaagactaatagattgctccttacgaggagacaggctggcccgccccgcagcgcacatgaggatcacccatgtgc

SEQ ID NO: 46 - DNA, 79 bp, Artificial Sequence
  Synthetic
ctttcaagactaatagattgctccttacgaggagacgtgttccagtttcctttagcgcacatgaggatcacccatgtgc

SEQ ID NO: 47 - DNA, 43 bp, Artificial Sequence
  Synthetic
aatagattgctccttacgaggagacaggctggcccgccccgca

SEQ ID NO: 48 - DNA, 43 bp, Artificial Sequence
  Synthetic
aatagattgctccttacgaggagacgtgttccagtttccttta

SEQ ID NO: 49 - DNA, 66 bp, Artificial Sequence
  Synthetic
gcacatgaggatcacccatgtgcaatagattgctccttacgaggagacaggctggcccgccccgca

SEQ ID NO: 50 - DNA, 66 bp, Artificial Sequence
  Synthetic
gcacatgaggatcacccatgtgcaatagattgctccttacgaggagacgtgttccagtttccttta

SEQ ID NO: 51 - DNA, 54 bp, Artificial Sequence
  Synthetic
ctttcaagactaatagattgctccttacgaggagacaggaatgcccgccagcgc

SEQ ID NO: 52 - DNA, 78 bp, Artificial  Sequence
  Synthetic
cgcacatgaggatcacccatgtgcctttcaagactaatagattgctccttacgaggagacaggaatgcccgccagcgc

SEQ ID NO: 53 - DNA, 56 bp, Artificial Sequence
  Synthetic
ctttcaagactaatagattgctccttacgaggagacaggctggcccgccccgcagt

SEQ ID NO: 54 - DNA, 56 bp, Artificial Sequence
  Synthetic
ctttcaagactaatagattgctccttacgaggagacgtgttccagtttcctttaca

SEQ ID NO: 55 - DNA, 56 bp, Artificial Sequence
  Synthetic
ctttcaagactaatagattgctccttacgaggagacaggaatgcccgccagcgcga

SEQ ID NO: 56 - DNA, 80 bp, Artificial Sequence
  Synthetic
cgcacatgaggatcacccatgtgcctttcaagactaatagattgctccttacgaggagacaggctggcccgccccgcagt

SEQ ID NO: 57 - DNA, 80 bp, Artificial Sequence
  Synthetic
cgcacatgaggatcacccatgtgcctttcaagactaatagattgctccttacgaggagacgtgttccagtttcctttaca

SEQ ID NO: 58 - DNA, 80 bp, Artificial Sequence
  Synthetic
cgcacatgaggatcacccatgtgcctttcaagactaatagattgctccttacgaggagacaggaatgcccgccagcgcga

SEQ ID NO: 59 - RNA, 55 nt, Artificial Sequence
  Synthetic
gcuuucaagacuaauagauugcuccuuacgaggagacguguuccaguuuccuuua

SEQ ID NO: 60 - RNA, 79 nt, Artificial Sequence
  Synthetic
gcgcacaugaggaucacccaugugccuuucaagacuaauagauugcuccuuacgaggagacguguuccaguuuccuuua

SEQ ID NO: 61 - RNA, 80 nt, Artificial Sequence
  Synthetic
gcuuucaagacugcgcacaugaggaucacccaugugcaauagauugcuccuuacgaggagacguguuccaguuuccuuua

SEQ ID NO: 62 - RNA, 77 nt, Artificial Sequence
  Synethtic
cuuucaagacuaauagauugcuccuuacaacaugaggaucacccauguugcgaggagacguguuccaguuuccuuua

SEQ ID NO: 63 - RNA, 81 nt, Artificial Sequence
  Synthetic
gcuuucaagacuaauagauugcuccuuacgaggagacguguuccaguuuccuuuagcgcacaugaggaucacccaugmugc

SEQ ID NO: 64 - RNA, 42 nt, Artificial Sequence
  Synthetic
aauagauugcuccuuacgaggagacguguuccaguuuccuuu

SEQ ID NO: 65 - RNA, 66 nt, Artificial Sequence
  Synthetic
gcacaugaggaucacccaugugcaauagauugcuccuuacgaggagacguguuccaguuuccuuua

SEQ ID NO: 66 - DNA, 18 bp, Homo sapiens
taaaggaaactggaacac

SEQ ID NO: 67 - RNA, 38 nt, Artificial Sequence
  Synthetic
  N_region (38)..(38): N is A, C, G, or U
  misc_feature (38)..(38): n is a, c, g, or u
gcuuucaagacuaauagauugcuccuuacgaggagacn

SEQ ID NO: 68 - RNA, 24 nt, Artificial Sequence
  Synthetic
  N_region (24)..(24): N is A, C, G, or U
  misc_feature (24)..(24): n is a, c, g, or u
agaaauccgucuuucauugacggn

SEQ ID NO: 69 - RNA, 24 nt, Artificial Sequence
  Synthetic
  N_region (24)..(24): N is A, C, G. or U
  misc_feature (24)..(24): n is a, c, g, or u
agaaauccgucuuucauugacggn

SEQ ID NO: 70 - RNA, 38 nt, Artificial Sequence; identical to SEQ ID NO: 67
  Synthetic
  N_region (38)..(38): N is A, C, G. or U
  misc_feature (38)..(38): n is a, c, g, or u

SEQ ID NO: 71 - RNA, 24 nt, Artificial Sequence
  Synthetic
  N_region (24)..(24): N is A, C, G, or U
  misc_feature (24)..(24): n is a, c, g, or u
agaaauccgucuuucauugacggn

SEQ ID NO: 72 - RNA, 55 nt, Artificial Sequence
  Synthetic
  misc_feature (1)..(2): 2′-O-methyl
  misc_feature (53)..(54): 2′-O-methyl
gcuuucaagacuaauagauugcuccuuacgaggagacaggcuggcccgccccgca

SEQ ID NO: 73 - RNA, 55 nt, Artificial Sequence; identical to SEQ ID NO: 59
  Synthetic
  misc_feature (1)..(2): 2′′-O-methyl
  misc_feature (53)..(54): 2′′-O-methyl

SEQ ID NO: 74 - RNA, 79 nt, Artificial Sequence
  Synthetic
  misc_feature (1)..(2): 2′-O-methyl
  misc_feature (77)..(78): 2′-O-methyl
gcgcacaugaggaucacccaugugccuuucaagacuaauagauugcuccuuacgaggagacaggcuggcccgccccgca

SEQ ID NO: 75 - RNA, 79 nt, Artificial Sequence; identical to SEQ ID NO: 60
  Synthetic
  misc_feature (1)..(2): 2′-O-methyl
  misc_feature (77)..(78): 2′-O-methyl

SEQ ID NO: 76 - RNA, 80 nt, Artificial Sequence
  Synthetic
  misc_feature (1)..(2): 2′-O-methyl
  misc_feature (78)..(79): 2′-O-methyl
gcuuucaagacugcgcacaugaggaucacccaugugcaauagauugcuccuuacgaggagacaggcuggcccgccccgca

SEQ ID NO: 77 - RNA, 80 nt, Artificial Sequence; identical to SEQ ID NO: 61
  Synthetic
  misc_feature (1)..(2): 2′-O-methyl
  misc_feature (78)..(79): 2′-O-methyl

SEQ ID NO: 78 - RNA, 80 nt, Artificial Sequence
  Synthetic
  misc_feature (1)..(2): 2′-O-methyl
  misc_feature (78)..(79): 2′-O-methyl
gcuuucaagacuaauagauugcuccuuacgaggagacaggcuggcccgccccgcagcgcacaugaggaucacccaugugc

SEQ ID NO: 79 - RNA, 80 nt, Artificial Sequence
  Synthetic
  misc_feature (1)..(2): 2′-O-methyl
  misc_feature (78)..(79): 2′-O-methyl
gcuuucaagacuaauagauugcuccuuacgaggagacguguuccaguuuccuuuagcgcacaugaggaucacccaugugc

SEQ ID NO: 80 - RNA, 43 nt, Artificial Sequence
  Synthetic
  misc_feature (1)..(2): 2′-O-methyl
  misc_feature (41)..(42): 2′-O-methyl
aauagauugcuccuuacgaggagacaggcuggcccgccccgca

SEQ ID NO: 81 - RNA, 42 nt, Artificial Sequence; identical to SEQ ID NO: 64
  Synthetic
  misc_feature (1)..(2): 2′-O-methyl
  misc_feature (40)..(41): 2′-O-methyl

SEQ ID NO: 82 - RNA, 66 nt, Artificial Sequence
  Synthetic
  misc_feature (1)..(2): 2′-O-methyl
  misc_feature (64)..(65): 2′-O-methyl
gcacaugaggaucacccaugugcaauagauugcuccuuacgaggagacaggcuggcccgccccgca

SEQ ID NO: 83 - RNA, 66 nt, Artificial Sequence; identical to SEQ ID NO: 65
  Synthetic
  misc_feature (1)..(2): 2′-O-methyl
  misc_feature (64)..(65): 2′-O-methyl

SEQ ID NO: 84 - RNA, 55 nt, Artificial Sequence
  Synthetic
  misc_feature (1)..(2)
  misc_feature (53)..(54)
gcuuucaagacuaauagauugcuccuuacgaggagacaggaaugcccgccagcgc

SEQ ID NO: 85 - RNA, 79 nt, Artificial Sequence
  Synthetic
  misc_feature (1)..(2): 2′-O-methyl
  misc_feature (77)..(78): 2′-O-methyl
gcgcacaugaggaucacccaugugccuuucaagacuaauagauugcuccuuacgaggagacaggaaugcccgccagcgc

SEQ ID NO: 86 - RNA, 83 nt, Artificial Sequence
  Synthetic
  misc_feature (1)..(2): 2′-O-methyl
  N_region (66)..(83): N is A, C, G, or U
  misc_feature (66)..(80): n is a, c, g, or u
  misc_feature (81)..(82): 2′-O-methyl
  misc_feature (83)..(83): n is a, c, g, or u
ucucgcgcacaugaggaucacccaugugccuuucaagacuaauagauugcuccuuacgaggagacnnnnnnnnnnnnnnnnnn

SEQ ID NO: 87 - RNA, 89 nt, Artificial Sequence
  Synthetic
  misc_feature (1)..(2): 2′-O-methyl
  N_region (72)..(89): N is A, C, G, or U
  misc_feature (72)..(86): n is a, c, g, or u
  misc_feature (87)..(88): 2′-O-methyl
  misc_feature (89)..(89): n is a, c, g, or u
gcgcacaugaggaucacccaugugcucucucucuccuuucaagacuaauagauugcuccuuacgaggagacnnnnnnnnnnnnnnnnnn

SEQ ID NO: 88 - RNA, 93 nt, Artificial Sequence
  Synthetic
  misc_feature (1)..(2): 2′-O-methy;
  N_region (76)..(93): N is A, C, G. or  U
  misc_feature (76)..(90): n is a, c, g, or u
  misc_feature (91)..(92): 2′-O-methy;
  misc_feature (93)..(93): n is a, c, g, or u
ucucgcgcacaugaggaucacccaugugcucucucucuccuuucaagacuaauagauugcuccuuacgaggagacnnnnnnnnnnnnnnnnnn

SEQ ID NO: 89 - RNA, 83 nt, Artificial Sequence
  Synthetic
  misc_feature (1)..(2): 2′-O-methyl
  N_region (41)..(58): N is A, C, G, or U
  misc_feature (41)..(58): n is a, c, g, or u
  misc_feature (81)..(82): 2′-O-methyl
ucuccuuucaagacuaauagauugcuccuuacgaggagacnnnnnnnnnnnnnnnnnngcgcacaugaggaucacccaugugc

SEQ ID NO: 90 - RNA, 89 nt, Artificial Sequence
  Synthetic
  misc_feature (1)..(2): 2′-O-methyl
  N_region (37)..(54): N is A, C, G. or U
  misc_feature (37)..(54): n is a, c, g, or u
  misc_feature (87)..(88): 2′-O-methyl
cuuucaagacuaauagauugcuccuuacgaggagacnnnnnnnnnnnnnnnnnngcucucucucucgcacaugaggaucacccaugugc

SEQ ID NO: 91 - RNA, 93 nt, Artificial Sequence
  Synthetic
  misc_feature (1)..(2): 2′-O-methyl
  misc_feature (41)..(58): n is a, c, g, or u
  N_region (51)..(68): N is A, C, G, or U
  misc_feature (91)..(92): 2′-O-methyl
ucuccuuucaagacuaauagauugcuccuuacgaggagacnnnnnnnnnnnnnnnnnngcucucucucucgcacaugaggaucacccaugugc

SEQ ID NO: 92 - RNA, 59 nt, Artificial Sequence
  Synthetic
  misc_feature (1)..(2): 2′-O-methyl
  misc_feature (57)..(58): 2′-O-methyl
ucucgcuuucaagacuaauagauugcuccuuacgaggagacguguuccaguuuccuuua

SEQ ID NO: 93 - RNA, 83 nt, Artificial Sequence
  Synthetic
ucucgcgcacaugaggaucacccaugugccuuucaagacuaauagauugcuccuuacgaggagacguguuccaguuuccuuua

SEQ ID NO: 94 - RNA, 89 nt, Artificial Sequence
  Synthetic
  misc_feature (1)..(2): 2′-O-methyl
  misc_feature (87)..(88): 2′-O-methyl
gcgcacaugaggaucacccaugugcucucucucuccuuucaagacuaauagauugcuccuuacgaggagacguguuccaguuuccuuua

SEQ ID NO: 95 - RNA, 93 nt, Artificial Sequence
  Synthetic
  misc_feature (1)..(2): 2′-O-methyl
  misc_feature (91)..(92): 2′-O-methyl
ucucgcgcacaugaggaucacccaugugcucucucucuccuuucaagacuaauagauugcuccuuacgaggagacguguuccaguuuccuuua

SEQ ID NO: 96 - RNA, 83 nt, Artificial Sequence
  Synthetic
  misc_feature (1)..(2): 2′-O-methyl
  misc_feature (56)..(57): 2′-O-methyl
  misc_feature (81)..(82): 2′-O-methyl
ucuccuuucaagacuaauagauugcuccuuacgaggagacguguuccaguuuccuuuagcgcacaugaggaucacccaugugc

SEQ ID NO: 97 - RNA, 89 nt, Artificial Sequence
  Synthetic
  misc_feature (1)..(2): 2′-O-methyl
  misc_feature (52)..(53): 2′-O-methyl
  misc_feature (87)..(88): 2′-O-methyl
cuuucaagacuaauagauugcuccuuacgaggagacguguuccaguuuccuuuagcucucucucucgcacaugaggaucacccaugugc

SEQ ID NO: 98 - RNA, 93 nt, Artificial Sequence
  Synthetic
  misc_feature (1)..(2): 2′-O-methyl
  misc_feature (56)..(57): 2′-O-methyl
  misc_feature (91)..(92): 2′-O-methyl
ucuccuuucaagacuaauagauugcuccuuacgaggagacguguuccaguuuccuuuagcucucucucucgcacaugaggaucacccaugugc

SEQ ID NO: 99 - RNA, 58 nt, Artificial Sequence
  Synthetic
  misc_feature (1)..(2): 2′-O-methyl
  misc_feature (57)..(58): 2′-O-methyl
mgmcuuucaagacuaauagauugcuccuuacgaggagacaggcuggcccgccccgcag

SEQ ID NO: 100 - RNA, 81 nt, Artificial Sequence
  Synthetic
  misc_feature (1)..(2): 2′-O-methyl
  misc_feature (79)..(80): 2′-O-methyl
gcgcacaugaggaucacccaugugccuuucaagacuaauagauugcuccuuacgaggagacaggcuggcccgccccgcagu

SEQ ID NO: 101 - RNA, 57 nt, Artificial Sequence
  Synthetic
  misc_feature (1)..(2): 2′-O-methyl
  misc_feature (55)..(56): 2′-O-methyl
gcuuucaagacuaauagauugcuccuuacgaggagacguguuccaguuuccuuuaca

SEQ ID NO: 102 - RNA, 81 nt, Artificial Sequence
  Synthetic
  misc_feature (1)..(2): 2′-O-methyl
  misc_feature (79)..(80): 2′-O-methyl
gcgcacaugaggaucacccaugugccuuucaagacuaauagauugcuccuuacgaggagacguguuccaguuuccuuuaca

SEQ ID NO: 103 - RNA, 57 nt, Artificial Sequence
  Synthetic
  misc_feature (1)..(2): 2′-O-methyl
  misc_feature (55)..(56): 2′-O-methyl
gcuuucaagacuaauagauugcuccuuacgaggagaccucucccgcucugcacccuc

SEQ ID NO: 104 - RNA, 81 nt, Artificial Sequence
  Synthetic
  misc_feature (1)..(2): 2′-O-methyl
  misc_feature (79)..(80): 2′-O-methyl
gcgcacaugaggaucacccaugugccuuucaagacuaauagauugcuccuuacgaggagaccucucccgcucugcacccuc

SEQ ID NO: 105 - RNA, 57 nt, Artificial Sequence
  Synthetic
  misc_feature (1)..(2): 2′-O-methyl
  misc_feature (56)..(57): 2′-O-methyl
gcuuucaagacuaauagauugcuccuuacgaggagacaggaaugcccgccagcgcga

SEQ ID NO: 106 - RNA, 81 nt, Artificial Sequence
  Synthetic
  misc_feature (1)..(2): 2′-O-methyl
  misc_feature (79)..(80): 2′-O-methyl
gcgcacaugaggaucacccaugugccuuucaagacuaauagauugcuccuuacgaggagacaggaaugcccgccagcgcga

SEQ ID NO: 107 - RNA, 81 nt, Artificial Sequence; identical to SEQ ID NO: 102
  Synthetic

SEQ ID NO: 108 - RNA, 81 nt, Artificial Sequence; identical to SEQ ID NO: 102
  Synthetic
  misc_feature (1)..(1): 2′-O-methyl
  misc_feature (81)..(81): 2′-O-methyl

SEQ ID NO: 109 - RNA, 81 nt, Artificial Sequence; identical to SEQ ID NO: 102
  Synthetic
  misc_feature (1)..(2): 2′-O-methyl
  misc_feature (80)..(81): 2′-O-methyl

SEQ ID NO: 110 - RNA, 81 nt, Artificial Sequence; identical to SEQ ID NO: 102
  Synthetic
  misc_feature (1)..(3): 2′-O-methyl
  misc_feature (79)..(81): 2′-O-methyl

SEQ ID NO: 111 - RNA, 81 nt, Artificial Sequence; identical to SEQ ID NO: 102
  Synthetic
  misc_feature (1)..(3): 2′-O-methyl
  misc_feature (79)..(81): 2′-O-methyl

SEQ ID NO: 112 - RNA, 57 nt, Artificial Sequence; identical to SEQ ID NO: 101
  Synthetic
  misc_feature (1)..(3): 2′-O-methyl
  misc_feature (55)..(57): 2′-O-methyl

SEQ ID NO: 113 - RNA, 81 nt, Artificial Sequence; identical to SEQ ID NO: 106
  Synthetic

SEQ ID NO: 114 - RNA, 81 nt, Artificial Sequence; identical to SEQ ID NO: 106
  Synthetic
  misc_feature (1)..(1): 2′-O-methyl
  misc_feature (81)..(81): 2′-O-methyl

SEQ ID NO: 115 - RNA, 81 nt, Artificial Sequence; identical to SEQ ID NO: 106
  Synthetic
  misc_feature (1)..(2): 2′-O-methyl
  misc_feature (80)..(81): 2′-O-methyl

SEQ ID NO: 116 - RNA, 81 nt, Artificial Sequence; identical to SEQ ID NO: 106
  Synthetic
  misc_feature (1)..(3): 2′-O-methyl
  misc_feature (79)..(81): 2′-O-methyl

SEQ ID NO: 117 - RNA, 81 nt, Artificial Sequence; identical to SEQ ID NO: 106
  Synthetic
  misc_feature (1)..(3): 2′-O-methyl
  misc_feature (79)..(81): 2′-O-methyl

SEQ ID NO: 118 - RNA, 57 nt, Artificial Sequence; identical to SEQ ID NO: 105
  Synthetic
  misc_feature (1)..(3): 2′-O-methyl
  misc_feature (55)..(57): 2′-O-methyl

SEQ ID NO: 119 - RNA, 55 nt, Artificial Sequence
  Synthetic
  misc_feature (1)..(2): 2′-O-methyl
  misc_feature (53)..(54): 2′-O-methyl
gcuuucaagacuaauagauugcuccuuacgaggagaccucucccgcucugcaccc

SEQ ID NO: 120 - RNA, 79 nt, Artificial Sequence
  Synthetic
  misc_feature (1)..(2): 2′-O-methyl
  misc_feature (77)..(78): 2′-O-methyl
gcgcacaugaggaucacccaugugccuuucaagacuaauagauugcuccuuacgaggagaccucucccgcucugcaccc

SEQ ID NO: 121 - DNA, 2829 bp, Artificial Sequence
  Synthetic
atggtcgacgggagcgggccggcagctaaacgggtgaagttggacagtggtggaattaaacctacagtttctcagtttcttacccctggttttaagctgataagaaaccatagtcggacggctggacttaagctgaagaatgagggcgaagaggcatgcaagaagttcgtacgggagaacgaaattcccaaagatgaatgtccaaactttcaaggtggacccgcaatcgcgaacattatagccaagagtcgcgaatttaccgagtgggaaatatatcaaagttcactggcgatccaagaggtgattttcaccttgccgaaggataagctgcccgagcctatactcaaggaagaatggcgcgcccaatggttgagcgaacacggcctcgatacggtgccttacaaggaagctgccggacttaatttgataattaagaacgcggtcaacacttacaaaggggtccaggtgaaagtcgataataagaataagaacaacctggccaaaatcaaccgcaagaacgaaatcgcgaaattgaacggcgaacaagaaatcagcttcgaagagatcaaagccttcgatgataaaggatatctcctgcaaaagccaagtccgaataagagcatatattgctaccaaagcgtgtctccaaagccattcataacctctaaataccataacgtgaatctgcccgaagaatatatcggctactaccgcaagtcaaacgagcccatcgttagtccctatcaattcgatagattgcgaatcccaattggcgaacccggatatgtaccaaaatggcagtatacctttctgtctaagaaagagaataagcggagaaagctctccaagcggattaagaatgttagtcctattcttgggataatatgcattaagaaagactggtgcgtattcgatatgaggggcctgctcagaacgaaccactggaagaaataccataaaccgacagattctatcaatgacctcttcgattatttcactggagaccctgtaatcgacacgaaagcgaacgtcgtccgattcagatataaaatggaaaatggcattgttaattacaagccggtgcgcgaaaagaaaggcaaggaacttttggaaaacatatgtgatcaaaatgggagctgtaagttggccactgtggccgttggtcaaaacaacccagtggcaattggactgtttgaacttaagaaagtaaatggtgaacttaccaaaaccttgatttcacggcatcctactccgatcgacttttgtaataaaattacggcttacagggagcggtatgataagctcgaatccagcatcaagttggatgccataaagcaattgacatctgagcaaaagatcgaagttgataactataacaataattttacccctcaaaacactaagcagatagtgtgcagcaagctcaatatcaatccaaacgaccttccttgggataaaatgatttctgggactcatttcattagcgagaaagcccaagtcagtaataaatcagaaatatacttcacatctaccgataaggggaaaactaaggacgtaatgaagagcgactacaagtggtttcaagactataaaccaaaactgtcaaaggaagtaagggacgcactcagcgatattgaatggcggcttaggagagaaagtcttgaatttaacaaattgagtaaatcacgggaacaagatgcacggcaactggccaattggatctcttccatgtgtgatgttatcggaatagccaacctggtgaagaagaacaatttctttggtggaagcggcaagagggaaccggggtgggacaacttctataaaccgaagaaggagaatcgatggtggatcaacgcaattcataaagctctcacagaactctctcaaaacaaagggaaaagagtgattctcttgccagcaatgagaacatctatcacatgccctaaatgtaagtactgtgacagcaagaaccggaacggcgagaagttcaattgtctgaagtgtggcatagaactcaacgcagccattgatgttgctaccgaaaatctcgcgaccgttgctattaccgcgcaaagtatgcctaaacccacctgtgagaggagtggtgatgccaagaagcccgtacgtgcacgaaaggcaaaggcgccagaatttcatgacaaactcgcgccctcatacacagttgtcttgcgcgaagctgttagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaatga

SEQ ID NO: 122 - DNA, 2829 bp, Artificial Sequence
  Synthetic
atggtcgacggcagcggccccgccgccaagagagtgaagctggacagcggcggcatcaagcccaccgtgtctcagttcctgacccccggcttcaagctgatcagaaaccacagcagaactgccggtctgaaattgaagaacgagggcgaggaggcctgcaagaagttcgtgagagaaaacgaaatccccaaggacgagtgccccaacttccaaggcggccccgccatcgccaacatcatcgccaagagcagagagttcacggagtgggagatctatcagagcagcctggccatccaagaggtgatcttcaccctgcccaaggacaagctgcccgagcccatcctgaaggaggagtggagagctcagtggctgagcgagcacggcctggacaccgtgccctacaaggaggccgccggcctgaatcttatcatcaagaacgccgtgaacacctacaagggcgtgcaagtgaaggtggacaacaagaacaagaacaacctggccaagatcaacagaaagaacgagatcgccaagctgaatggcgaacaagagatcagcttcgaggagatcaaggccttcgacgacaagggctacctgctgcagaagcctagccccaataagagcatctactgctatcagagcgtgagccccaagcccttcatcacaagcaagtaccacaacgtgaacctgcccgaggagtacatcggctactacagaaagagcaacgagcccatcgtgagcccctatcagttcgacagactgagaatccccatcggcgagcccggctacgtgcccaagtggcagtacaccttcctgagcaagaaggaaaacaaaagaagaaagctcagcaagagaatcaagaacgtgagccccatcctgggcatcatctgcatcaagaaggactggtgcgtgttcgacatgagaggcctgctgagaaccaaccactggaagaagtaccacaagcccaccgacagcatcaacgacctgttcgactatttcaccggcgaccccgtgatcgacaccaaggccaacgtggtgagattcagatacaagatggagaacggcatcgtgaactacaagcccgttcgcgagaaaaagggcaaggagctgctggagaacatctgcgatcagaacggcagctgcaagctggcgactgtggccgtggggcagaacaatcccgtggccatcggcctgttcgagctgaagaaggtaaacggcgagctgaccaagaccctgatcagcagacaccccacccccatcgacttctgcaacaagatcaccgcctacagagagagatacgacaagctggagtctagcatcaagctggacgccatcaagcagctgacaagcgagcagaagatcgaggtggacaactacaacaacaacttcacccctcagaacaccaagcagatcgtgtgcagcaagctgaacatcaaccccaacgacctgccctgggacaagatgatcagcggcacccacttcatttccgagaaggcccaagtgagcaacaagagcgagatctacttcacaagcaccgacaagggaaagaccaaagacgtgatgaagagcgactataagtggttccaagactacaaacccaaactaagcaaagaggtgcgggacgccctgagcgacatcgagtggagactgagaagagagagcctggagttcaacaaattatcgaaatctcgggagcaagacgctagacagctggccaactggatcagcagcatgtgcgacgtgatcggcatcgccaacctggtgaagaagaacaacttcttcggcggcagcggcaagagagagcccggctgggacaacttctacaagcccaagaaagagaacagatggtggatcaacgccatccacaaggccctgaccgagctgtctcagaacaagggcaagagagtgatcctgctgcccgccatgagaacaagcatcacctgccccaagtgcaagtactgcgacagcaagaacagaaacggcgagaagttcaactgcctgaagtgcggcatcgagctgaacgccgccatcgacgtggccaccgagaacctagctaccgtggccatcaccgctcagagcatgcccaagcccacctgcgagagaagcggcgacgccaagaaacccgtcagagctcgcaaggccaaggcccccgagttccatgacaagctggccccaagctacaccgtggtgctgagagaggccgtgagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaatga

SEQ ID NO: 123 - DNA, 2208 bp, Artificial Sequence
  Synthetic
atggtcgacgggagcgggccggcagctaaacgggtgaagttggacagtggtggaattaaacctacagtttctcagtttcttacccctggttttaagctgataagaaaccatagtcggacggctggacttaagctgaagaatgagggcgaagaggcatgcaagaagttcgtacgggagaacgaaattcccaaagatgaatgtccaaactttcaaggtggacccgcaatcgcgaacattatagccaagagtcgcgaatttaccgagtgggaaatatatcaaagttcactggcgatccaagaggtgattttcaccttgccgaaggataagctgcccgagcctatactcaaggaagaatggcgcgcccaatggttgagcgaacacggcctcgatacggtgccttacaaggaagctgccggacttaatttgataattaagaacgcggtcaacacttacaaaggggtccaggtgaaagtcgataataagaataagaacaacctggccaaaatcaaccgcaagaacgaaatcgcgaaattgaacggcgaacaagaaatcagcttcgaagagatcaaagccttcgatgataaaggatatctcctgcaaaagccaagtccgaataagagcatatattgctaccaaagcgtgtctccaaagccattcataacctctaaataccataacgtgaatctgcccgaagaatatatcggctactaccgcaagtcaaacgagcccatcgttagtccctatcaattcgatagattgcgaatcccaattggcgaacccggatatgtaccaaaatggcagtatacctttctgtctaagaaagagaataagcggagaaagctctccaagcggattaagaatgttagtcctattcttgggataatatgcattaagaaagactggtgcgtattcgatatgaggggcctgctcagaacgaaccactggaagaaataccataaaccgacagattctatcaatgacctcttcgattatttcactggagaccctgtaatcgacacgaaagcgaacgtcgtccgattcagatataaaatggaaaatggcattgttaattacaagccggtgcgcgaaaagaaaggcaaggaacttttggaaaacatatgtgatcaaaatgggagctgtaagttggccactgtggccgttggtcaaaacaacccagtggcaattggactgtttgaacttaagaaagtaaatggtgaacttaccaaaaccttgatttcacggcatcctactccgatcgacttttgtaataaaattacggcttacagggagcggtatgataagctcgaatccagcatcaagttggatgccataaagcaattgacatctgagcaaaagatcgaagttgataactataacaataattttacccctcaaaacactaagcagatagtgtgcagcaagctcaatatcaatccaaacgaccttccttgggataaaatgatttctgggactcatttcattagcgagaaagcccaagtcagtaataaatcagaaatatacttcacatctaccgataaggggaaaactaaggacgtaatgaagagcgactacaagtggtttcaagactataaaccaaaactgtcaaaggaagtaagggacgcactcagcgatattgaatggcggcttaggagagaaagtcttgaatttaacaaattgagtaaatcacgggaacaagatgcacggcaactggccaattggatctcttccatgtgtgatgttatcggaatagagaacctggtgaagaagaacaatttctttggtggaagcggcaagagggaaccggggtgggacaacttctataaaccgaagaaggagaatcgatggtggatcaacgcaattcataaagctctcacagaactctctcaaaacaaagggaaaagagtgattctcttgccagcaatgagaacatctatcacatgccctaaatgtaagtactgtgacagcaagaaccggaacggcgagaagttcaattgtctgaagtgtggcatagaactcaacgcagacattgatgttgctaccgaaaatctcgcgaccgttgctattaccgcgcaaagtatgcctaaacccacctgtgagaggagtggtgatgccaagaagcccgtacgtgcacgaaaggcaaaggcgccagaatttcatgacaaactcgcgccctcatacacagttgtcttgcgcgaagctgtttaatga

SEQ ID NO: 124 - DNA, 2208 bp, Artificial Sequence
  Synthetic
atggtcgacgggagcgggccggcagctaaacgggtgaagttggacagtggtggaattaaacctacagtttctcagtttcttacccctggttttaagctgataagaaaccatagtcggacggctggacttaagctgaagaatgagggcgaagaggcatgcaagaagttcgtacgggagaacgaaattcccaaagatgaatgtccaaactttcaaggtggacccgcaatcgcgaacattatagccaagagtcgcgaatttaccgagtgggaaatatatcaaagttcactggcgatccaagaggtgattttcaccttgccgaaggataagctgcccgagcctatactcaaggaagaatggcgcgcccaatggttgagcgaacacggcctcgatacggtgccttacaaggaagctgccggacttaatttgataattaagaacgcggtcaacacttacaaaggggtccaggtgaaagtcgataataagaataagaacaacctggccaaaatcaaccgcaagaacgaaatcgcgaaattgaacggcgaacaagaaatcagcttcgaagagatcaaagccttcgatgataaaggatatctcctgcaaaagccaagtccgaataagagcatatattgctaccaaagcgtgtctccaaagccattcataacctctaaataccataacgtgaatctgcccgaagaatatatcggctactaccgcaagtcaaacgagcccatcgttagtccctatcaattcgatagattgcgaatcccaattggcgaacccggatatgtaccaaaatggcagtatacctttctgtctaagaaagagaataagcggagaaagctctccaagcggattaagaatgttagtcctattcttgggataatatgcattaagaaagactggtgcgtattcgatatgaggggcctgctcagaacgaaccactggaagaaataccataaaccgacagattctatcaatgacctcttcgattatttcactggagaccctgtaatcgacacgaaagcgaacgtcgtccgattcagatataaaatggaaaatggcattgttaattacaagccggtgcgcgaaaagaaaggcaaggaacttttggaaaacatatgtgatcaaaatgggagctgtaagttggccactgtggatgttggtcaaaacaacccagtggcaattggactgtttgaacttaagaaagtaaatggtgaacttaccaaaaccttgatttcacggcatcctactccgatcgacttttgtaataaaattacggcttacagggagcggtatgataagctcgaatccagcatcaagttggatgccataaagcaattgacatctgagcaaaagatcgaagttgataactataacaataattttacccctcaaaacactaagcagatagtgtgcagcaagctcaatatcaatccaaacgaccttccttgggataaaatgatttctgggactcatttcattagcgagaaagcccaagtcagtaataaatcagaaatatacttcacatctaccgataaggggaaaactaaggacgtaatgaagagcgactacaagtggtttcaagactataaaccaaaactgtcaaaggaagtaagggacgcactcagcgatattgaatggcggcttaggagagaaagtcttgaatttaacaaattgagtaaatcacgggaacaagatgcacggcaactggccaattggatctcttccatgtgtgatgttatcggaatagccaacctggtgaagaagaacaatttctttggtggaagcggcaagagggaaccggggtgggacaacttctataaaccgaagaaggagaatcgatggtggatcaacgcaattcataaagctctcacagaactctctcaaaacaaagggaaaagagtgattctcttgccagcaatgagaacatctatcacatgccctaaatgtaagtactgtgacagcaagaaccggaacggcgagaagttcaattgtctgaagtgtggcatagaactcaacgcagacattgatgttgctaccgaaaatctcgcgaccgttgctattaccgcgcaaagtatgcctaaacccacctgtgagaggagtggtgatgccaagaagcccgtacgtgcacgaaaggcaaaggcgccagaatttcatgacaaactcgcgccctcatacacagttgtcttgcgcgaagctgtttaatga

SEQ ID NO: 125 - DNA, 79 bp, Artificial Sequence
  Synthetic
gcgcacatgaggatcacccatgtgcagaaatccgtctttcattgacggacagatggggctggacaatttttcccccttt

SEQ ID NO: 126 - DNA, 79 bp, Artificial Sequence
  Synthetic
agaaatccgtctttcattgacggacagatggggctggacaatttttccccctttgcgcacatgaggatcacccatgtgc

SEQ ID NO: 127 - DNA, 54 bp, Artificial Sequence
  Synthetic
agaaatccgtctttcattgacggacagatggggctggacaatttttcccccttt

SEQ ID NO: 128 - DNA, 79 bp, Artificial Sequence
  Synthetic
gcgcacatgaggatcacccatgtgcagaaatccgtctttcattgacggcagtttcctttacagggccagcgggctggaa

SEQ ID NO: 129 - DNA, 79 bp, Artificial Sequence
  Synthetic
agaaatccgtctttcattgacggcagtttcctttacagggccagcgggctggaagcgcacatgaggatcacccatgtgc

SEQ ID NO: 130 - DNA, 54 bp, Artificial Sequence
  Synthetic
agaaatccgtctttcattgacggcagtttcctttacagggccagcgggctggaa

SEQ ID NO: 131 - DNA, 79 bp, Artificial Sequence
  Synthetic
gcgcacatgaggatcacccatgtgcagaaatccgtctttcattgacggaggaatgcccgccagcgcgacgcctccactt

SEQ ID NO: 132 - DNA, 79 bp, Artificial Sequence
  Synthetic
agaaatccgtctttcattgacggaggaatgcccgccagcgcgacgcctccacttgcgcacatgaggatcacccatgtgc

SEQ ID NO: 133 - DNA, 54 bp, Artificial Sequence
  Synthetic
agaaatccgtctttcattgacggaggaatgcccgccagcgcgacgcctccactt

SEQ ID NO: 134 - DNA, 2829 bp, Artificial Sequence; identical to SEQ ID NO: 121
  Synthetic

SEQ ID NO: 135 - RNA, 78 nt, Artificial Sequence; identical to SEQ ID NO: 30
  Synthetic
  misc_feature (1)..(2): 2′-O-methyl
  N_region (61)..(78): N is A, C, G, or U
  misc_feature (61)..(76): n is a, c, g, or u
  misc_feature (77)..(78): 2′-O-methyl

SEQ ID NO: 136 - RNA, 80 nt, Artificial Sequence
  Synthetic
  misc_feature (1)..(2): 2′-O-methyl
  N_region (61)..(80): N is A, C, G or U
  misc_feature (61)..(77): n is a, c, g, or u
  misc_feature (78)..(79): 2′-O-methyl
  misc_feature (80)..(80): n is a, c, g, or u
gcgcacaugaggaucacccaugugcuuucaagacuaauagauugcuccuuacgaggagacnnnnnnnnnnnnnnnnnnnn

SEQ ID NO: 137 - RNA, 78 nt, Artificial Sequence; identical to SEQ ID NO: 30
  Synthetic
  N_region (61)..(78): N is A, C, G.or U
  misc_feature (61)..(78): n is a, c, g, or u

SEQ ID NO: 138 - RNA, 80 nt, Artificial Sequence; identical to SEQ ID NO: 136
  Synthetic
  N_region (61)..(80): N is A, C, G. or U
  misc_feature (61)..(80): n is a, c, g, or u

SEQ ID NO: 139 - DNA, 990 bp, Artificial Sequence
  Synthetic
atggcccccaagaagaagcggaaagtgatggagccggaggcttttcagcgcaactttgaccctcgggaatttgccgcctgtacactcctcttgtatgagatccactgggacaataacacatctagaaattggtgtacgaataagcctgggctccacgctgaggagaatttcttgcagatatttaatgagaaaattgacattaaacaggatacgccgtgctctataacatggttcctttcttggagcccctgttacccttgtagccaagcaataataaaattcttggaggcacacccgaatgtcagtctggagattaaggctgcgcggctgtatatgcatcaaatagactgtaacaaggagggactcagaaatctgggccggaatcgagtgtcaataatgaacctgcctgattataggcattgctggactacgtttgttgtgccaaggggagcaaacgaagattactggccacaagactttctgcctgcgatcacaaattactcccgagaactcgactccatactgcaggatgagctgaagacacccctgggcgacaccacacacacctctccaccttgcccagcaccagagctgctgggaggccctatggccagcaacttcacacagtttgtgctggtggataatggaggaaccggcgacgtgacagtggcaccatctaactttgccaatggcatcgccgagtggatcagctccaactctcggagccaggcctataaggtgacctgtagcgtgcggcagtctagcgcccagaatagaaagtatacaatcaaggtggaggtgcctaagggcgcctggagatcctacctgaacatggagctgaccatcccaatctttgccacaaattctgattgcgagctgatcgtgaaggccatgcagggcctgctgaaggacggcaaccctatcccaagcgccatcgccgccaatagcggaatctacacgcgtaaaatcagcctcgactgtgccttctag

SEQ ID NO: 140 - protein, 84 aa, Unknown
  Bacillus phage PBS2
MTNLSDIIEKETGKQLVIQESILMLPEEVEEVIGNKPESDILVHTAYDESTDENVMLLTSDAPEYKPWALVIQDSNGENKIKML

SEQ ID NO: 141 - DNA, 594 bp, Unknown
  hAID
atggatagcctgctgatgaaccggagaaagttcctgtatcagtttaagaatgtgcgctgggcaaagggcaggcgcgagacctacctgtgctatgtggtgaagcggagagattccgccacatccttctctctggactttggctacctgcggaacaagaatggctgccacgtggagctgctgttcctgagatacatctctgactgggatctggacccaggcaggtgttatcgcgtgacctggttcacaagctggtccccctgctacgattgtgcaaggcacgtggcagactttctgaggggaaacccaaatctgtccctgcggatcttcaccgccagactgtatttttgcgaggataggaaggcagagccagagggactgaggcgcctgcacagggccggcgtgcagatcgccatcatgaccttcaaggactacttttattgttggaacaccttcgtggagaatcacgagcggaccttcaaggcctgggagggactgcacgagaactccgtgcggctgtctagacagctgcggagaatcctgctgcctctgtacgaggtggacgatctgagggatgccttccgcaccctgggactg

SEQ ID NO: 142 - DNA, 69 bp, Artificial Sequence
  Synthetic
  misc_feature (1)..(2): 2′-O-methyl
  misc_feature (67)..(68): 2′-O-methyl
gcgcacaugaggaucacccaugugcuaauuucuacuaaguguagaucagcccgctggccctgtaaagga

SEQ ID NO: 143 - DNA, 86 bp, Artificial Sequence
  Synthetic
  misc_feature (1)..(2): 2′-O-methyl
  misc_feature (84)..(85): 2′-O-methyl
gcgcacaugaggaucacccaugugcgucaaaagacuuuuuaauaauuucuacuaaguguagaucagcccgctggccctgtaaagga

SEQ ID NO: 144 - DNA, 69 bp, Artificial Sequence
  Synthetic
  misc_feature (1)..(2): 2′-O-methyl
  misc_feature (67)..(68): 2′-O-methyl
uaauuucuacuaaguguagaucagcccgctggccctgtaaaggagcgcacaugaggaucacccaumugc

SEQ ID NO: 145 - DNA, 86 bp, Artificial Sequence
  Synthetic
  misc_feature (1)..(2): 2′-O-methyl
  misc_feature (84)..(85): 2′-O-methyl
gucaaaagacuuuuuaauaauuucuacuaaguguagaucagcccgctggccctgtaaaggagcgcacaugaggaucacccaugugc

SEQ ID NO: 146 - DNA, 61 bp, Artificial Sequence
  Synthetic
  misc_feature (1)..(2): 2′-O-methyl
  misc_feature (59)..(60): 2′-O-methyl
gucaaaagacuuuuuaauaauuucuacuaaguguagaucagcccgctggccctgtaaagga

SEQ ID NO: 147 - DNA, 597 bp, Artificial Sequence
  Synthetic
atggaggcatctccagcatccggtccaaggcatctcatggatccccatatcttcacctccaattttaataacggaatcgggcgccacaagacatacttgtgctatgaggtggaacgactggacaacggtacctccgtgaaaatggaccaacatcgcggatttctgcataatcaggctaaaaaccttctgtgtggattttatgggagacacgctgagctgagatttcttgacctggtcccgagcttacagctggacccagcccaaatctatcgcgtaacttggttcatcagctggagcccctgcttttccgccgggtgcgctggagaagtgcgggcgttcctgcaggaaaacacccacgtcagactgaggatttttgcagcacgcatctacgactatgattatctttacaaggaggcattacagatgttgcgcgatgccggagcccaagtaagcattatgacttatgatgagttcaaacactgttgggacacctttgtagaccaccagggctgcccctttcagccttgggatgggctcgacgagcacagccaggcactcagcggacgcctccgcgctatcctccagaaccagggtaac